(19) United States
(12) Patent Application Publication
(10) Pub. No.: US 2013/0253646 A1
(43) Pub. Date: Sep. 26, 2013

(54) SILK BASED IMPLANTABLE MEDICAL DEVICES AND METHODS FOR DETERMINING SUITABILITY FOR USE IN HUMANS

(71) Applicant: Allergan, Inc., Irvine, CA (US)

(72) Inventors: Gregory H. Altman, Arlington, MA (US); Enrico Mortarino, Hickory, NC (US); Raymond E. Olsen, Smithfield, UT (US)

(73) Assignee: Allergan, Inc., Irvine, CA (US)

(21) Appl. No.: 13/898,023

(22) Filed: May 20, 2013

Related U.S. Application Data
(63) Continuation-in-part of application No. 13/372,248, filed on Feb. 13, 2012.
Application No. 13/372,248 is a continuation-in-part of application No. 13/289,786, filed on Nov. 4, 2011.
(60) Provisional application No. 61/650,322, filed on May 22, 2012.

(51) Int. Cl. A61F 2/12

(52) U.S. Cl. CPC A61F 2/12
USPC 623/8

(57) ABSTRACT

Methods for determining suitability of an implantable silk scaffold for use in human soft tissue repair by implanting a silk scaffold in a quadruped. The silk scaffold is completely or essentially completely bioresorbed by twelve months after implantation, the silk scaffold (to the extent remaining) with ingrown tissue shows at least about a 60% strength increase by 12 months after implantation, and the thickness of the silk scaffold (to the extent remaining) with ingrown tissue increases by more than 100% by 12 months after implantation.

### CROSS REFERENCE

This application is a non-provisional of and claims the benefit of U.S. Provisional Patent Application No. 61/650,322, filed May 22, 2012, and is also a continuation-in-part of U.S. patent application Ser. No. 13/372,248, filed Feb. 13, 2012, which is a continuation-in-part of U.S. patent application No. 13/289,786, filed Nov. 4, 2011, the entire contents of each of which are incorporated herein in their entireties.

### FIELD OF THE INVENTION

The present invention relates to implantable medical devices made of or based on silk. More particularly the present invention relates to a implantable medical scaffold made of silk for use in soft tissue repair in humans, including for example in breast reconstruction, breast augmentation, abdominal surgery, hernia repair and facial surgery. The present invention also relates to in vivo animal models or methods for determining that the implantable medical device scaffolds are suitable for use in human cosmetic and surgical purposes, for example in breast reconstruction and/or breast augmentation surgeries and procedures.

BACKGROUND OF THE INVENTION

In the case of soft tissue repair, surgical meshes and scaffolds are widely used for breast and chest wall reconstruction, strengthening tissues, providing support for internal organs, and treating surgical or traumatic wounds. They are usually made of inert materials and polymers such as Teflon®, polypropylene, polyglycolic acid, polyester, polyglactin 910, etc., although a titanium mesh has been used in some spinal surgeries. But the use of tissue based materials such as acellular dermal matrix (ADM) from human and animal derived dermis is also becoming more popular.

Surgical mesh devices are typically biocompatible and can be made from bioresorbable and/or non-bioresorbable material. For example, Teflon®, polypropylene and polyester are biocompatible and non-bioresorbable while polyglycolic acid and polyglactin 910 are biocompatible and bioresorbable. ADM is processed by removing the cells and epidermis, if applicable, from the donor tissue and leaving only natural biologic components.

One application for soft tissue reconstruction that uses surgical mesh or ADM is breast reconstruction post mastectomy. The aim of breast reconstructive surgery is to restore a woman's breasts to a near normal appearance and shape following the surgical removal of a breast (mastectomy), a crucial step towards emotional healing in women who have been faced with losing their breast as a result of a medical condition such as breast cancer. According to the American Society of Plastic Surgeons (ASPS), approximately 60,000 surgical procedures occur in the U.S. related to non-cosmetic breast reconstruction. Internationally, that number surpasses 80,000 procedures when major industrialized countries are taken into consideration. A contoured, structural and tailored scaffold device designed to meet the unique needs of the breast reconstruction population where a massive loss of tissue occurs and which would work with the body's own immune process to restore the environment to a more natural state would provide a compassionate solution to a significant unmet need.

The breast reconstruction surgical procedure is commonly performed with two different methods, both using ADM as the preferred matrix. The main advantage of ADM over other available surgical meshes is the higher rate of revascularization, providing support and coverage of the defect while preventing infection and capsular contraction. The first method, a one stage reconstruction uses ADM to fully reconstruct the shape of the breast in conjunction with a breast implant at the time of the surgical procedure. The second method, a two stages reconstruction; the first stage consisting of the placement of (a) tissue expander(s) (at the time of mastectomy or later) with ADM to reconstruct the breast; follow by tissue expander expansion with saline solution to expand the muscle and skin tissue; the second and final stage consisting of the replacement of the tissue expander with an implant. In both procedures the pocket for the tissue expander or the implant is created by releasing the inferior origin of the pectoralis muscle and electocauterizing a subpectoral pocket. A sheet of ADM is centered over the defect and it is sutured to the inframammary fold with continuous or interrupted sutures. The tissue expander or implant is inserted and positioned inside the subpectoralis pocket created. The rest of the ADM is cut to the necessary shape and it is sutured to the inferior edge of the pectoralis muscles, while on the lateral border is sutured to the pectoralis and serratus muscles.

A breast reconstruction procedure alternative to the one described above using ADM is performed with autologous tissue such as TRAM flap. In this surgical procedure, the breast is reconstructed by using a portion of the abdomen tissue group that has been surgically removed, including the skin, the adipose tissue, minor muscles and connective tissue. This abdomen tissue group is taken from the patient's abdomen and transplanted onto the breast site using a similar method as described above with ADM.

The quality of the resulting reconstruction is impacted by subsequent treatment, e.g. post-mastectomy radiation weakens skin tissue, the amount of tissue available e.g. thinner women often lack sufficient tissue, and the overall health and habits, such as smoking, of the individual. Tissue expanders, balloon type devices, are frequently used in an attempt to stretch the harvested skin to accommodate the breast implant. However, harvested tissue has limitations in its ability to conform to the natural breast contour resulting in unacceptable results, including a less than ideal positioning or feel of the breast implant. A scaffold device that can be used as an internal scaffold to act as a “bra” to immediately support a geometrically complex implantation site at the time of surgery would ideally provide the body both the time and structure necessary for optimal healing.

Breast reconstruction in two stages with a tissue expander and ADM followed by the replacement of the tissue expander with an implant has become the most common technique adopted by surgeons. A main advantage is the lengthening of the pectoralis major muscle therefore preventing the commonly referred to as “window shading” after the muscle is released. Another main advantage is the control of the inframammary fold position and shape as well as the lateral breast border.

The use of ADM has advantages against the common surgical mesh devices by lowering the rate of capsular contraction and infection; however despite its low overall complication rate, the procedure is not without risk since ADM can generate a host inflammatory reaction and sometimes present infection. Also, it is very important to note that the properties of ADM are limited to the properties of the tissue that is harvested which can result in variability.

Thus, there is a need for a device or structure that can be used for reconstruction and support that overcomes the disadvantages of known methods and materials.

Furthermore, most biomaterials available today do not posses the mechanical integrity of high load demand applications (e.g., bone, ligaments, tendons, muscle) or the appropriate biological functionality; most biomaterials either degrade too rapidly (e.g., collagen, PLA, PGA, or related copolymers) or are non-degradable (e.g., polyesters, metal), where in either case, functional autologous tissue fails to develop and the patient suffers disability. In certain instances a biomaterial may misdirect tissue differentiation and development (e.g., spontaneous bone formation, tumors) because it lacks biocompatibility with surrounding cells and tissue. As well, a biomaterial that fails to degrade typically is associated with chronic inflammation, where such a response is actually detrimental to (i.e., weakens) surrounding tissue.

If properly designed, silk may offer new clinical options for the design of a new class of medical devices, scaffolds and matrices. Silk has been shown to have the highest strength of any natural fiber, and rivals the mechanical properties of synthetic high performance fibers. Silks are also stable at high physiological temperatures and in a wide range of pH, and are insoluble in most aqueous and organic solvents. Silk is a protein, rather than a synthetic polymer, and degradation products (e.g., peptides, amino acids) are biocompatible. Silk is non-mammalian derived and carries far less bioburden than other comparable natural biomaterials (e.g., bovine or porcine derived collagen).

Silk, as the term is generally known in the art, means a filamentous fiber product secreted by an organism such as a silkworm or spider. Silks produced from insects, namely (i) Bombyx mori silkworms, and (ii) the glands of spiders, typically Nephilia clavipes, are the most often studied forms of the material; however, hundreds to thousands of natural variants of silk exist in nature. Fibroin is produced and secreted by a silkworm's two silk glands. As fibroin leaves the glands, it is coated with sericin, a glue-like substance. However, spider silk is valued (and differentiated from silkworm silk) as it is produced as a single filament lacking any immunogenic contaminates, such as sericin.

Unfortunately, spider silk cannot be mass produced due to the inability to domesticate spiders; however, spider silk, as well as other silks can be cloned and recombinantly produced, but with extremely varying results. Often, these processes introduce bioburdens, are costly, cannot yield material in significant quantities, result in highly variable material properties, and are neither tightly controlled nor reproducible.

As a result, only silkworm silk has been used in biomedical applications for over 1,000 years. The Bombyx mori specie of silkworm produces a silk fiber (known as a “bave”) and uses the fiber to build its cocoon. The bave, as produced, includes two fibroin filaments or “broins”, which are surrounded with a coating of gum, known as sericin-the silk fibroin filament possesses significant mechanical integrity. When silk fibers are harvested for producing yarns or textiles, including sutures, a plurality of fibers can be aligned together, and the sericin is partially dissolved and then resolidified to create a larger silk fiber structure having more than two broins mutually embedded in a sericin coating.

As used herein, “fibroin” includes silkworm fibroin (i.e. from Bombyx mori) and fibroin-like fibers obtained from spiders (i.e. from Nephila clavipes). Alternatively, silk protein suitable for use in the present invention can be obtained from a solution containing a genetically engineered silk, such as from bacteria, yeast, mammalian cells, transgenic animals or transgenic plants. See, for example, WO 97/08315 and U.S. Pat. No. 5,245,012.

Silkworm silk fibers, traditionally available on the commercial market for textile and suture applications are often “degummed” and consist of multiple broins plied together to form a larger single multi-filament fiber. Degumming here refers to the loosening of the sericin coat surrounding the two broins through washing or extraction in hot soapy water. Such loosening allows for the plying of broins to create larger multifilament single fibers. However, complete extraction is often neither attained nor desired. Degummed silk often contains or is recoated with sericin and/or sericin impurities are introduced during plying in order to congeal the multifilament single fiber. The sericin coat protects the frail fibroin filaments (only ˜5 microns in diameter) from fraying during traditional textile applications where high-through-put processing is required. Therefore, degummed silk, unless explicitly stated as sericin-free, typically contain 10-26% (by weight) sericin.

Sericin is antigenic and elicits a strong immune, allergic or hyper-T-cell type (versus the normal mild “foreign body” response) response. Sericin may be removed (washed/extracted) from silk fibroin; however, removal of sericin from silk changes the ultrastructure of the fibroin fibers.

When typically referring to “silk” in the literature, it is inferred that the remarks are focused to the naturally-occurring and only available “silk” (i.e., sericin-coated fibroin fibers) which have been used for centuries in textiles and medicine. Medical grade silkworm silk is traditionally used in only two forms: (i) as virgin silk suture, where the sericin has not been removed, and (ii) the traditional more popular silk suture, or commonly referred to as black braided silk suture, where the sericin has been completely removed, but replaced with a wax or silicone coating to provide a barrier between the silk fibroin and the body tissue and cells. Presently, the only medical application for which silk is still used is in suture ligation, particularly because silk is still valued for it mechanical properties in surgery (e.g., knot strength and handlability).

Therefore, there also exists a need for silk or silk based implantable devices that are biocompatible and promote ingrowth of cells. Furthermore, there is a need for a model or method for evaluating and determining the performance of such devices and their suitability for use in humans.

### SUMMARY

The present invention relates to a surgical mesh (also referred to herein as a surgical scaffold) comprised of silk that is knitted, multi-filament, and bioengineered. It is mechanically strong, biocompatible, and long-term bioresorbable. As a feature of the scaffold of the present invention, the sericin-extracted silkworm fibroin fibers retain their native protein structure and have not been dissolved and reconstituted.

The surgical scaffold of the present invention is a sterile scaffold that is supplied in a variety of shapes and sizes ready for use in open surgical procedures or in laparoscopic procedures. The device is flexible and well-suited for delivery through a laparoscopic trocar due to its strength, tear resistance, suture retention, and ability to be cut in any direction. The surgical scaffold of the present invention provides immediate physical and mechanical stabilization of a tissue defect through the strength and porous (scaffold-like) construction of its mesh.

The present invention relates to a number of silk-based surgical mesh or scaffold designs. The surgical mesh or scaffold of the present invention is indicated for use as a transitory scaffold for soft tissue support and repair to reinforce deficiencies where weakness or voids exist that require the addition of material to obtain the desired surgical outcome. In another aspect of the present invention, an implantable scaffold to bridge and mechanically reinforce the void created with the insertion and filling of a breast tissue expander posterior to the pectoralis muscle in humans, by performing a similar implantation procedure deep to the latissimus dorsi muscle in four-legged animals such as sheep and pigs. The tissue expander is used to gradually enlarge the space beneath the muscle and the overlaying fascia tissue to accommodate the subsequent implantation of a permanent breast implant, as commonly performed after a total mastectomy procedure.

Preferably, the biodegradable silk medical device (scaffold) of the present invention is a biocompatible, non-woven, knit, multi-filament silk fabric or mesh. A woven material is made by weaving. Woven fabrics are classified as to weave or structure according to the manner in which warp and weft cross each other. The three main types of weaves (woven fabrics) are plain, twill, and satin. On the other hand a knitted fabric is generally softer and more supple than a woven fabric because its thread is treated differently. A knit or knitted fabric is made by using needles (such as for example the needles of a single or double bed knit machine) to pull threads up through the preceding threads to thereby make the fabric (explained in more detail supra). All the silk fabrics within the scope of the present invention are knit or knitted (for example warp or weft classification) silk fabrics. Woven (weaved) silk fabric, woven textiles and woven fabrics are not within the scope of the present invention. The silk fabric of the present invention can have can have an antibiotic coating.

The present invention encompasses a method for determining suitability of an implantable silk scaffold for use in human soft tissue repair, the method comprising the step of implanting a silk scaffold in a quadruped. The quadruped can be a sheep or a pig. The method can further comprising the step of evaluating the silk scaffold as a support structure for soft tissue in a human. The silk scaffold can maintain at least 90% of its time zero strength at one month in vivo after implantation. The silk scaffold can maintain at least 90% of its time zero strength at three months in vivo after implantation. The silk scaffold can maintains at least 90% of its time zero strength at six months in vivo after implantation. The silk scaffold can substantially maintain its time zero (i.e. at time of implantation) strength throughout its duration in vivo. Additionally, the thickness of the scaffold can increase with time in vivo due to tissue ingrowth. The scaffold can be implanted to simulate implantation in a human breast reconstruction or augmentation procedure. And the scaffold can be implanted without regard to side orientation of the scaffold.

The present invention also includes a method of evaluating in vivo a medical device in a quadruped animal model, the method comprising the step of implanting a quadruped with a tissue expander and a silk scaffold to support soft tissue. This method can further comprise suturing the silk scaffold to a sub-latissimus dorsi muscle and a chest wall of the quadruped.

Additionally, the present invention encompasses an animal model system for determining suitability of an implantable silk scaffold for use in human soft tissue repair, the animal model system comprising a silk scaffold, and a quadruped having a muscle for providing internal support for the silk scaffold. The quadruped can be is a sheep or a pig and the muscle can be a sub-latissimus dorsi muscle.

Finally, the present invention encompasses a method of supporting a breast tissue or a breast implant in a patient comprising obtaining a silk scaffold modeled in an animal system comprising a quadruped, and implanting the silk scaffold in a human for a breast augmentation or a breast reconstruction procedure.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DRAWINGS

The present invention will become more fully understood from the detailed description and the accompanying drawings, which are not necessarily to scale, wherein:

FIG. 1A is a photograph of a pattern layout for a silk-based scaffold design in accordance with the present invention.

FIGS. 1B and 1C illustrate an example pattern layout for the scaffold design of FIG. 1A including all pattern and ground bars according to aspects of the present invention.

FIGS. 1D and 1E illustrate an example pattern layout for a double needle bed mesh or scaffold according to aspects of the present invention from FIG. 1B for ground bar #4.

FIGS. 1F and 1G illustrate an example pattern layout for a double needle bed mesh or scaffold according to aspects of the present invention from FIG. 1B for pattern bar #5.

FIGS. 1H and 1I illustrate an example pattern layout for a double needle bed mesh or scaffold according to aspects of the present invention from FIG. 1B for ground bar #7.

FIG. 1J illustrates an example pattern simulation for a double needle bed mesh demonstrated in FIG. 1B according to aspects of the present invention.

FIG. 2A is a photograph of a pattern layout for a silk-based scaffold design in accordance with the present invention.

FIGS. 2B and 2C illustrate an example pattern layout for the scaffold design of FIG. 2A including all pattern and ground bars according to aspects of the present invention.

FIGS. 2D and 2E are enlarged views of the example pattern layout and ground bars of FIG. 2B.

FIGS. 3A and 3B illustrate an example pattern layout for a double needle bed mesh or scaffold according to aspects of the present invention from FIG. 2B for ground bar #4.

FIGS. 3C and 3D are enlarged views of the example pattern layout and ground bars of FIG. 2B.

FIGS. 4A and 4B illustrate an example pattern layout for a double needle bed mesh or scaffold according to aspects of the present invention from FIG. 2B for pattern bar #5.

FIGS. 4C and 4D are enlarged views of the example pattern layout and ground bars of FIG. 2B.

FIGS. 5A and 5B illustrate an example pattern layout for a double needle bed mesh according to aspects of the present invention from FIG. 2B for ground bar #7.

FIGS. 5C and 5D are enlarged views of the example pattern layout and ground bars of FIG. 2B.

FIG. 6 illustrates an example pattern simulation for a double needle bed mesh demonstrated in FIG. 2B according to aspects of the present invention.

FIG. 7A is a photograph of a pattern layout for a silk-based scaffold design in accordance with the present invention.

FIGS. 7B and 7C illustrate an example pattern layout for a silk-based scaffold design of FIG. 7A in accordance with the present invention including all pattern and ground bars according to aspects of the present invention.

FIGS. 7D and 7E are enlarged views of the example pattern layout and ground bars of FIG. 7B.

FIGS. 8A and 8B illustrate an example pattern layout for a double needle bed scaffold or mesh according to aspects of the present invention from FIG. 7B for ground bar #2.

FIGS. 8C and 8D are enlarged views of the example pattern layout and ground bars of FIG. 7B.

FIGS. 9A and 9B illustrate an example pattern layout for a double needle bed mesh or scaffold according to aspects of the present invention from FIG. 7B for pattern bar #4.

FIGS. 9C and 9D are enlarged views of the example pattern layout and ground bars of FIG. 7B.

FIGS. 10A and 10B illustrate an example pattern layout for a double needle bed mesh or scaffold according to aspects of the present invention from FIG. 7B for pattern bar #5.

FIGS. 10C and 10D are enlarged views of the example pattern layout and ground bars of FIG. 7B.

FIGS. 11A and 11B illustrate an example pattern layout for a double needle bed mesh or scaffold according to aspects of the present invention from FIG. 7B for ground bar #7.

FIGS. 11C and 11D are enlarged views of the example pattern layout and ground bars of FIG. 7B.

FIG. 12 illustrates an example pattern simulation for a double needle bed mesh demonstrated in FIG. 7B according to aspects of the present invention.

FIG. 13A is a photograph of a pattern layout for a silk-based scaffold design in accordance with the present invention.

FIGS. 13B and 13C illustrate an example pattern layout for the silk-based scaffold design of FIG. 13A in accordance with the present invention including all pattern and ground bars according to aspects of the present invention.

FIGS. 13D and 13E are enlarged views of the example pattern layout and ground bars of FIG. 13B.

FIG. 14A and 14B illustrate an example pattern layout for a double needle bed mesh or scaffold according to aspects of the present invention from FIG. 13B for ground bar #4.

FIGS. 14C and 14D are enlarged views of the example pattern layout and ground bars of FIG. 13B.

FIGS. 15A and 15B illustrate an example pattern layout for a double needle bed mesh or scaffold according to aspects of the present invention from FIG. 13B for pattern bar #5.

FIGS. 15C and 15D are enlarged views of the example pattern layout and ground bars of FIG. 13B.

FIGS. 16A and 16B illustrate an example pattern layout for a double needle bed mesh or scaffold according to aspects of the present invention from FIG. 13B for ground bar #7.

FIGS. 16C and 16D are enlarged views of the example pattern layout and ground bars of FIG. 13B.

FIG. 17 illustrates an example pattern simulation for a double needle bed scaffold demonstrated in FIG. 13B according to aspects of the present invention.

FIG. 18A is a photograph of a pattern layout for a silk-based scaffold design in accordance with the present invention.

FIGS. 18B and 18C illustrate an example pattern layout for the silk-based scaffold design of FIG. 18A in accordance with the present invention including all pattern and ground bars according to aspects of the present invention.

FIGS. 18D and 18E are enlarged views of the example pattern layout and ground bars of FIG. 18B.

FIGS. 19A and 19B illustrate an example pattern layout for a double needle bed mesh or scaffold according to aspects of the present invention from FIG. 18B for ground bar #4.

FIGS. 19C and 19D are enlarged views of the example pattern layout and ground bars of FIG. 18B.

FIGS. 20A and 20B illustrate an example pattern layout for a double needle bed mesh or scaffold according to aspects of the present invention from FIG. 18B for pattern bar #5.

FIGS. 20C and 20D are enlarged views of the example pattern layout and ground bars of FIG. 18B.

FIGS. 21A and 21B illustrate an example pattern layout for a double needle bed mesh or scaffold according to aspects of the present invention from FIG. 18B for ground bar #7.

FIGS. 21C and 21D are enlarged views of the example pattern layout and ground bars of FIG. 18B.

FIG. 22 illustrates an example pattern simulation for a double needle bed scaffold demonstrated in FIG. 18B according to aspects of the present invention.

FIG. 23A is a photograph of a pattern layout for a silk-based scaffold design in accordance with the present invention.

FIGS. 23B and 23C illustrate an example pattern layout for the silk-based scaffold design of FIG. 23A in accordance with the present invention including all pattern and ground bars according to aspects of the present invention.

FIGS. 23D and 23E are enlarged views of the example pattern layout and ground bars of FIG. 23B.

FIGS. 24A and 24B illustrate an example pattern layout for a double needle bed mesh or scaffold according to aspects of the present invention from FIG. 23B for ground bar #4.

FIGS. 24C and 24D are enlarged views of the example pattern layout and ground bars of FIG. 23B.

FIGS. 25A and 25B illustrate an example pattern layout for a double needle bed mesh or scaffold according to aspects of the present invention from FIG. 23B for pattern bar #5.

FIGS. 25C and 25D are enlarged views of the example pattern layout and ground bars of FIG. 23B.

FIGS. 26A and 26B illustrate an example pattern layout for a double needle bed mesh or scaffold according to aspects of the present invention from FIG. 23B for ground bar #7.

FIGS. 26C and 26D are enlarged views of the example pattern layout and ground bars of FIG. 23B.

FIG. 27 illustrates an example pattern simulation for a double needle bed scaffold demonstrated in FIG. 23B according to aspects of the present invention.

FIG. 28A illustrates a side profile of a human breast with implanted scaffold.

FIG. 28B illustrates a permanent implant having replaced a tissue expander in a human breast.

FIG. 29 illustrates a Latissimus dorsi sub-muscular tissue expander/breast implant location in a sheep.

FIG. 30 is a photograph of test article placement upon completion of the surgical procedure prior to incision closing in a sheep.

FIG. 31 is a photograph illustrating suturing the Latissimus dorsi muscle to the chest wall at three locations over the tissue expander in a sheep.

FIG. 32 is a layout of biomechanical and histological sample extraction from each sheep test animal article.

FIG. 33 illustrates a Latissimus dorsi sub-muscular ventro-cranial tissue expander placement in a pig for modeling in a human.

FIG. 34 illustrates a yarn comprised of silk bundles used in accordance with the present invention.

FIG. 35 is a photograph of SeriScaffold™ explanted one month after commencement of the sheep study set forth in Example 1, showing presence of tissue ingrowth at 1 month.

FIG. 36 is a photograph of SeriScaffold™ explanted twelve months after commencement of the sheep study set forth in Example 1, showing extensive presence of tissue ingrowth at 12 months, with little or no remaining SeriScaffold™ material.

FIGS. 37-45 illustrate other aspects of the invention.

### DESCRIPTION

The following detailed description of the embodiment(s) is merely exemplary in nature and is in no way intended to limit the invention, its application, or uses.

Mesh Designs

The present invention provides a biocompatible surgical silk scaffold device for use in soft tissue repair. Examples of soft tissue repair include breast reconstruction, hernia repair, cosmetic surgery, implementation of a bladder sling, or the like.

Although the present invention may employ a variety of polymer materials, a scaffold device using silk is the preferred material. Particular embodiments may be formed from Bombyx Mori silkworm silk fibroin. The raw silk fibers have a natural globular protein coating known as sericin, which may have antigenic properties and must be depleted before implantation. Accordingly, the yarn is taken through a depletion process. The depletion of sericin is further described, for example, by Gregory H. Altman et al., “Silk matrix for tissue engineered anterior cruciate ligaments,” Biomaterials 23 (2002), pp. 4131-4141, the contents of which are incorporated herein by reference. As a result, the silk material used in the device embodiments contains substantially no sensitizing agents, in so far as can be measured or predicted with standardized biomaterial test methods.

A surgical scaffold device according to aspect of the present invention is preferably created with knitting structures and relative machine parameters. The knitting structures involve variation in the methods of fabric formation such as the those classified as raschel knitting, warp knitting and weft knitting. The relative machine parameters may include, but are not limited to, variations such as yarn evolution, yarn design, loop size and length, number of courses and wales per unit measure, fabric take up rate, number of needles per unit measure and relative size, feed rate and relative tension applied to the yarn. Furthermore, post fabric formation treatment may enhance the characteristics of the scaffold's different regions. The fabric treatments may include, but are not limited to, finishing and surface coating process.

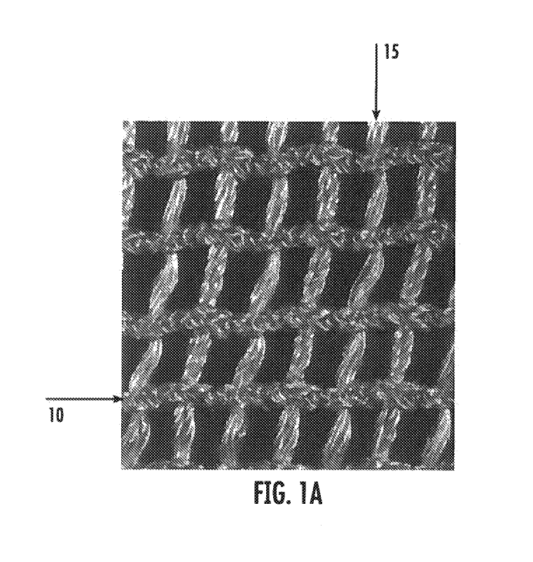

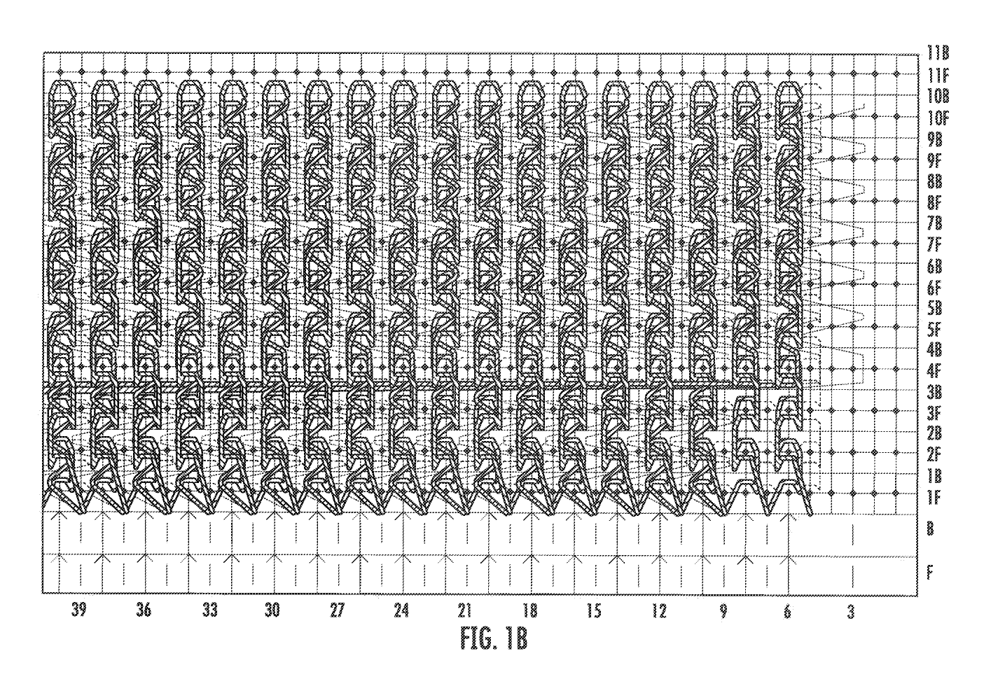

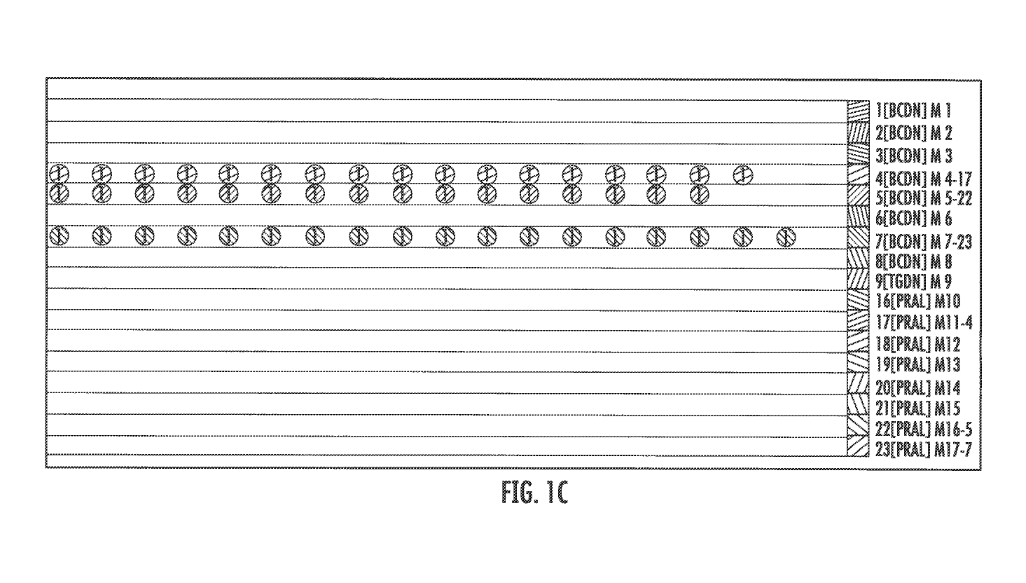

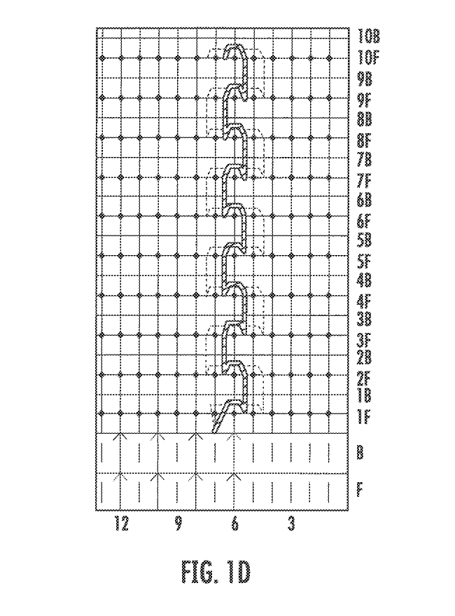

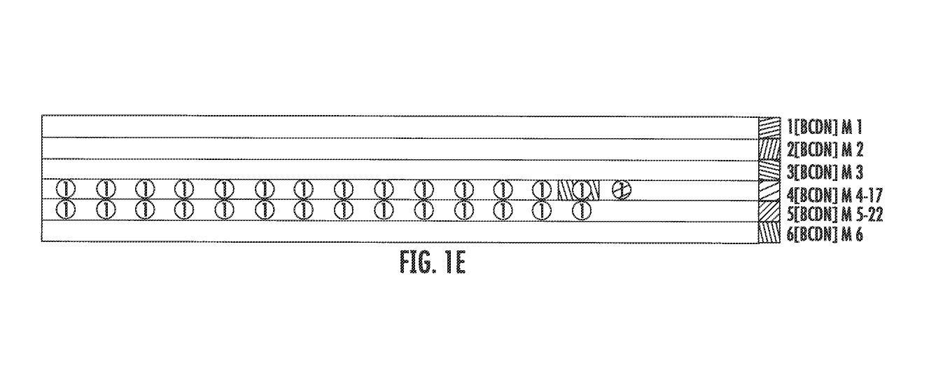

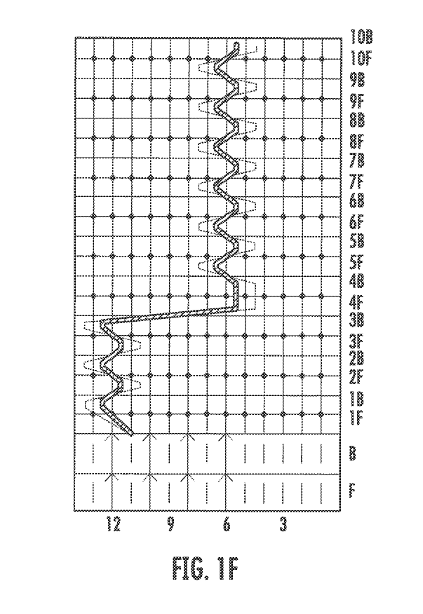

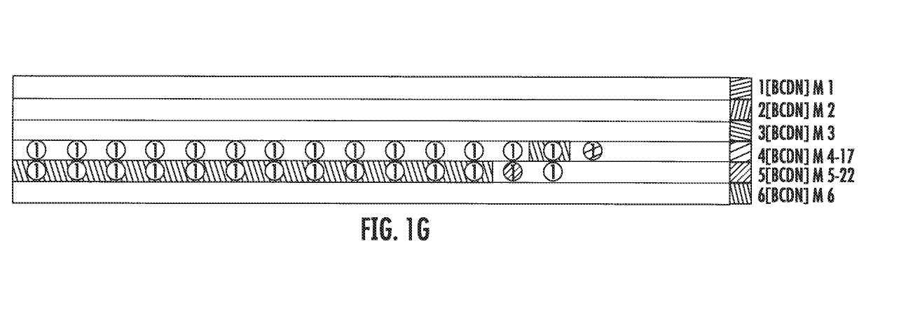

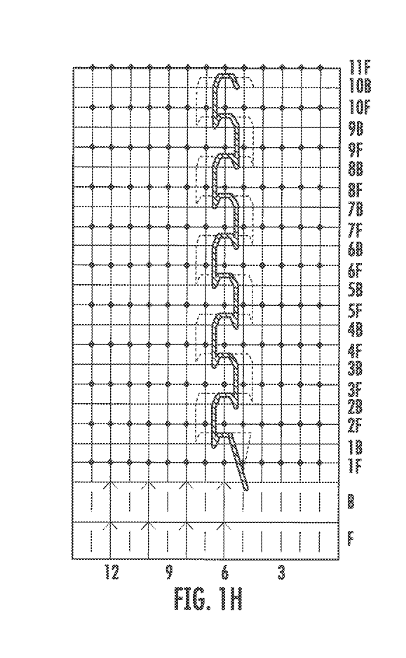

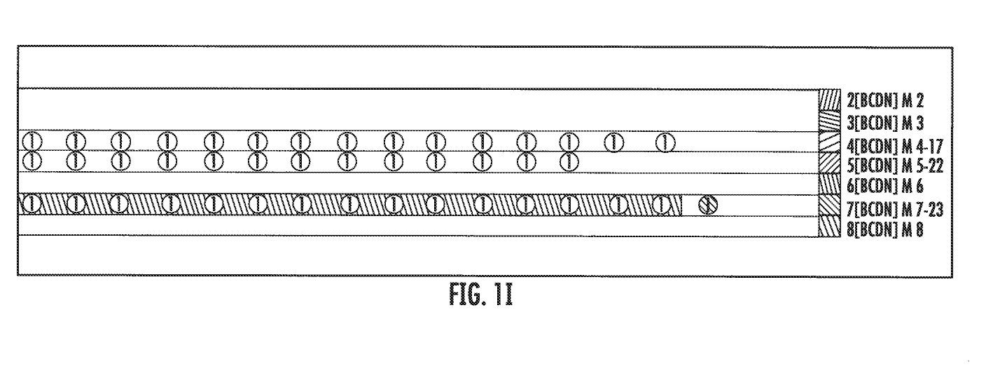

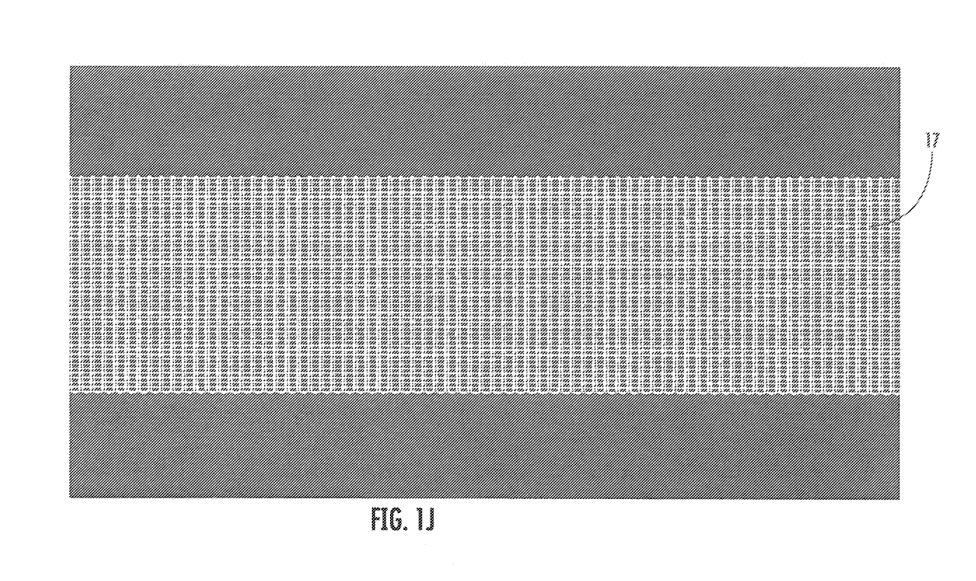

FIG. 1A is a photograph of a pattern layout for a silk-based mesh or scaffold design in accordance with the present invention. FIG. 1A shows the wale direction 10 and the course direction 15 and placement of the silk yarns in either the wale 10 or course 15 scaffold material direction or location. This scaffold in accordance with the present invention is preferably formed on a raschel knitting machine such as Comez DNB/EL-800-8B set up in 10 gg needle spacing by the use of three movements as shown in pattern layout in FIGS. 1B and 1C: two movements in the wale direction, the vertical direction within the fabric, and one movement in the course direction, the horizontal direction of the fabric. The movements in the wale direction occur on separate needle beds with alternate yarns; loops that occur on every course are staggered within repeat. The yarn follows a repeat pattern of 3/1-1/1-1/3-3/3 for one of the wale direction movements as shown in FIGS. 1D and 1E and 1/1-1/3-3/3-3/1 for the other wale direction movement as shown in FIGS. 1H and 1I. The interlacing of the loops within the fabric allows for one yarn to become under more tension than the other under stress, locking it around the less tensioned yarn; keeping the fabric from unraveling when cut. Preferably, the fabric is a node-lock fabric as described in detail in U.S. patent application Ser. Nos. 12/680,404 and 13/088,706, the entirety of which two applications is herein incorporated by reference. The other movement in the course direction as shown in FIG. 1F and 1G occurs in every few courses creating the porous design. These yarns follow a repeat pattern of 7/7-9/9-7/7-9/9-7/7-9/9/-1/1-1/1-3/3-1/1-3/3-1/1-3/3-1/1-3/3-1/1 for the course direction movement. The pattern simulation layout of this pattern is rendered with ComezDraw 3 software in FIG. 1J considering a yarn design made with 3 ends of Td (denier count) 20/22 raw silk twisted together in the S direction to form a ply with 20 tpi (turns per inch) and further combining three of the resulting ply with 10 tpi. In FIG. 1J The same yarn design is used for the movements occurring in the wale and course directions. The stitch density or pick count for the design in FIG. 1J is 34 picks per centimeter considering the total picks count for the technical front face and the technical back face of the fabric, or 17 picks per cm considering only on the face of the fabric. The operating parameters are not limited to those described in FIGS. 1B-1I, but just the optimum values for the specific yarn design used for the pattern simulation layout of FIG. 1J. In FIG. 1J item 17 is a simulated double needle bed mesh or scaffold.

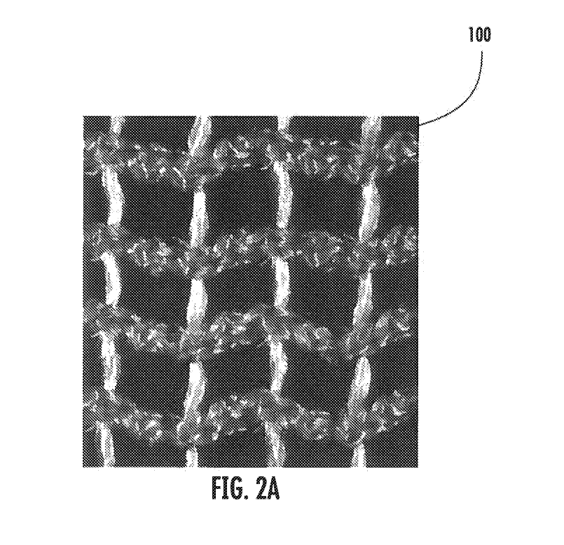

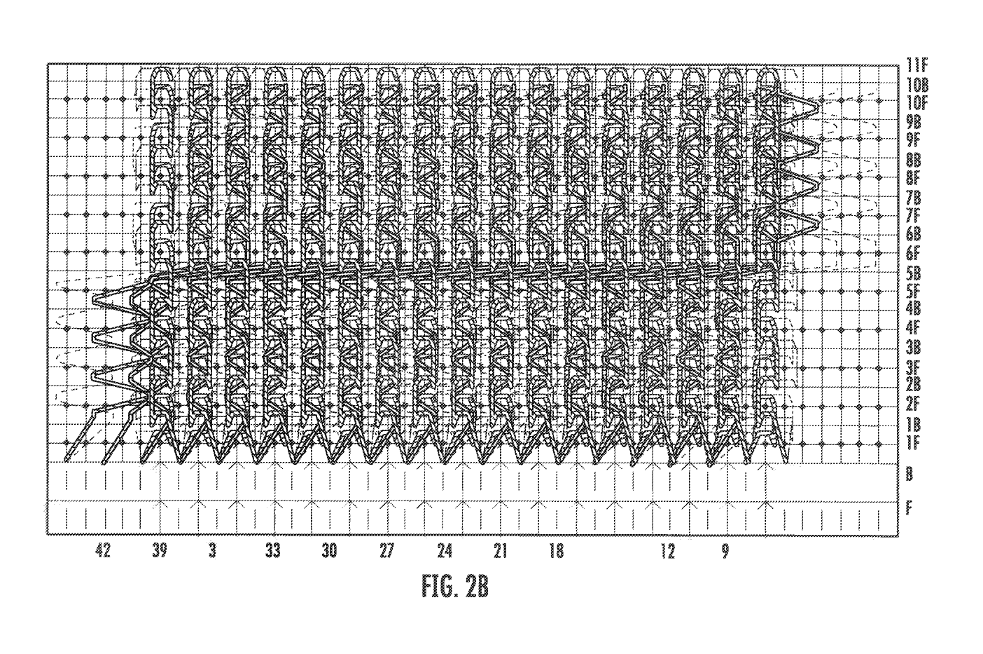

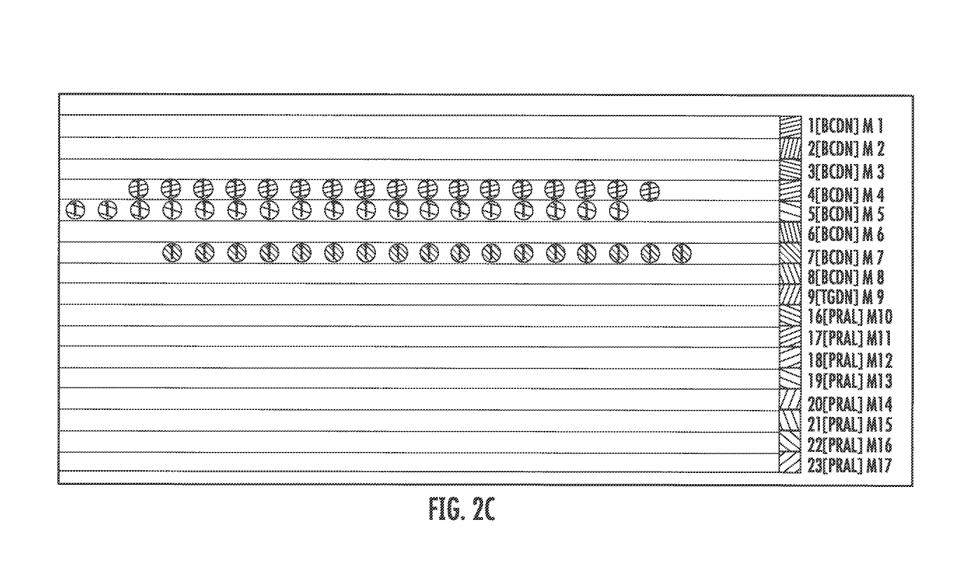

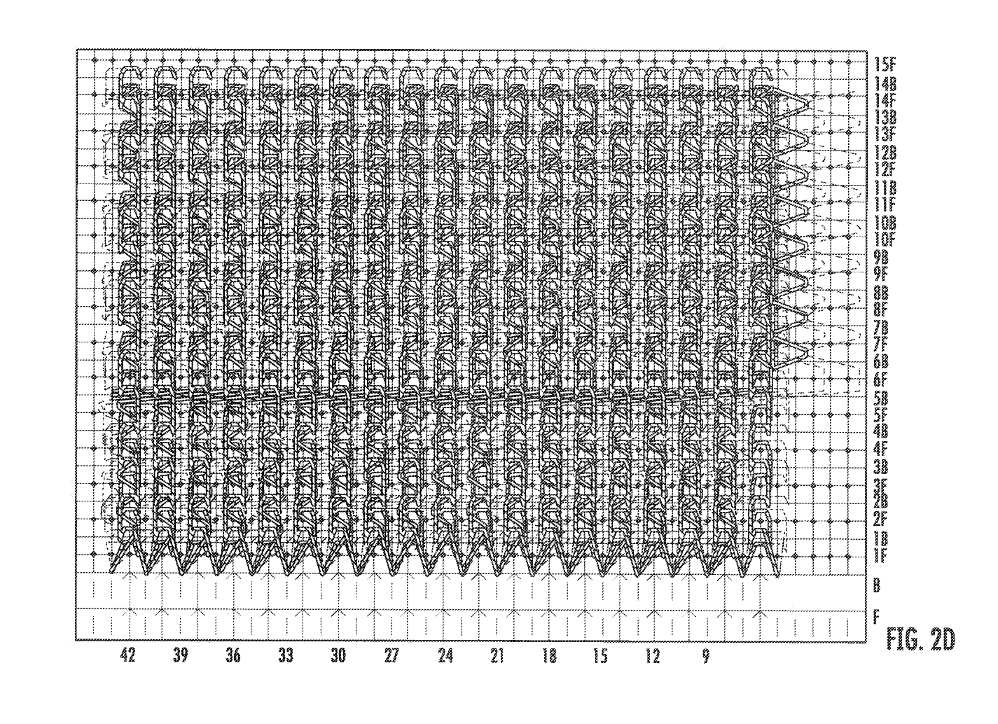

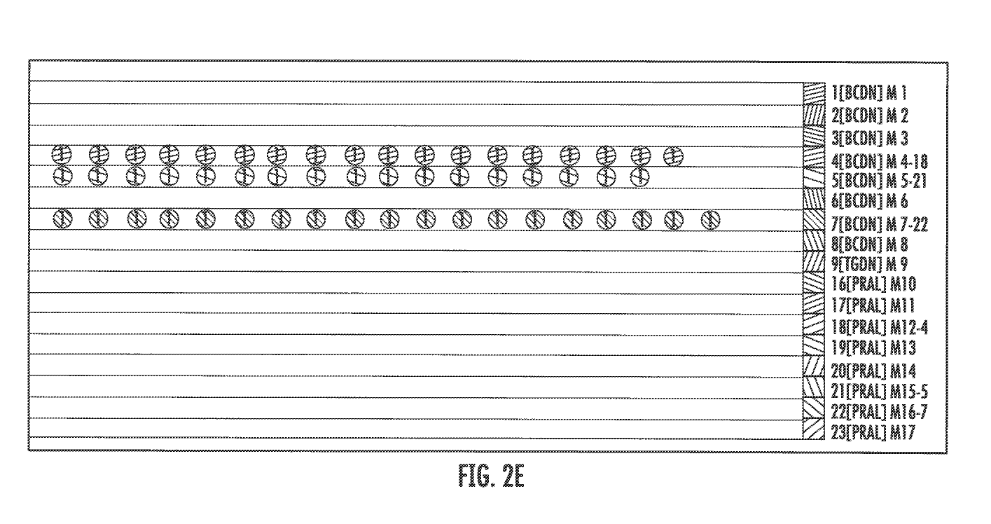

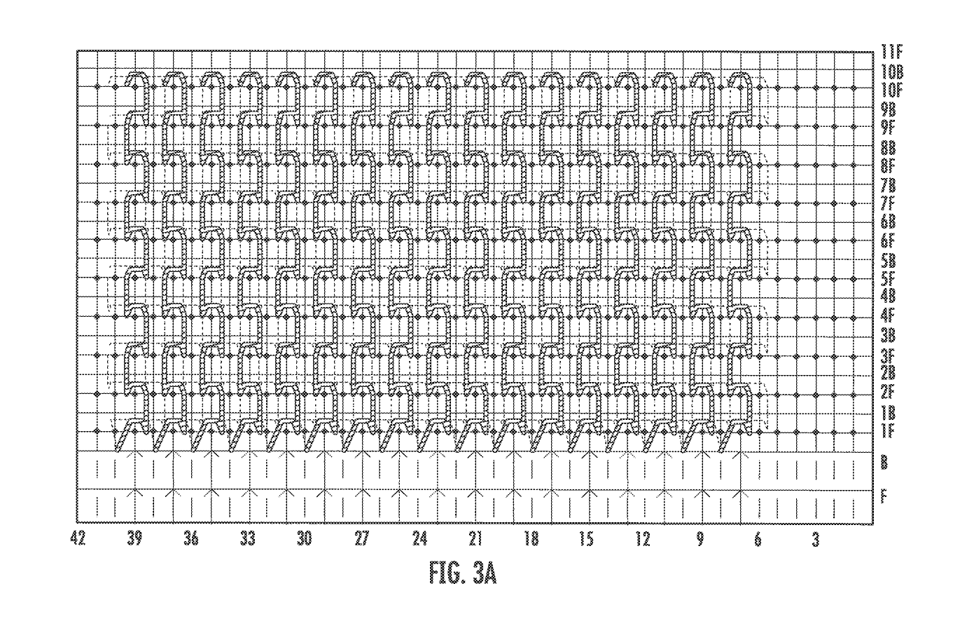

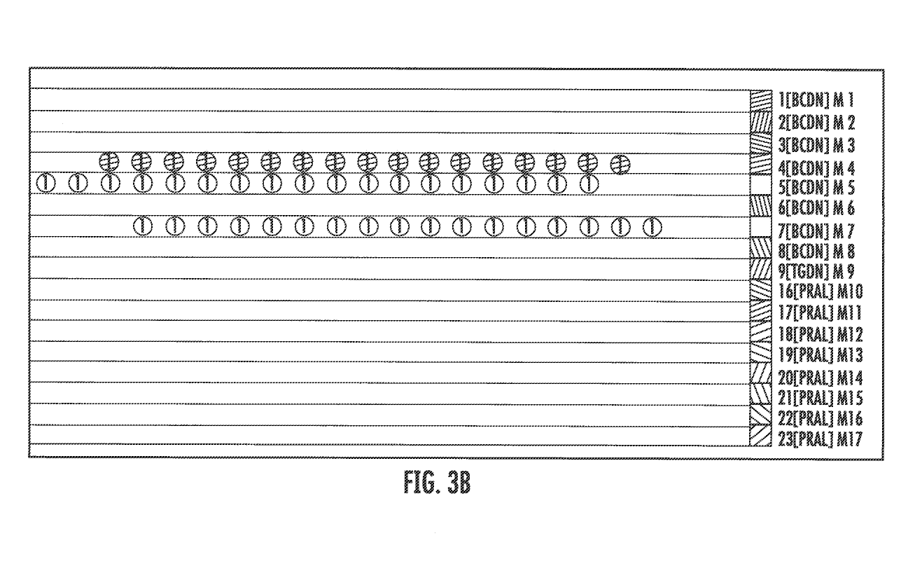

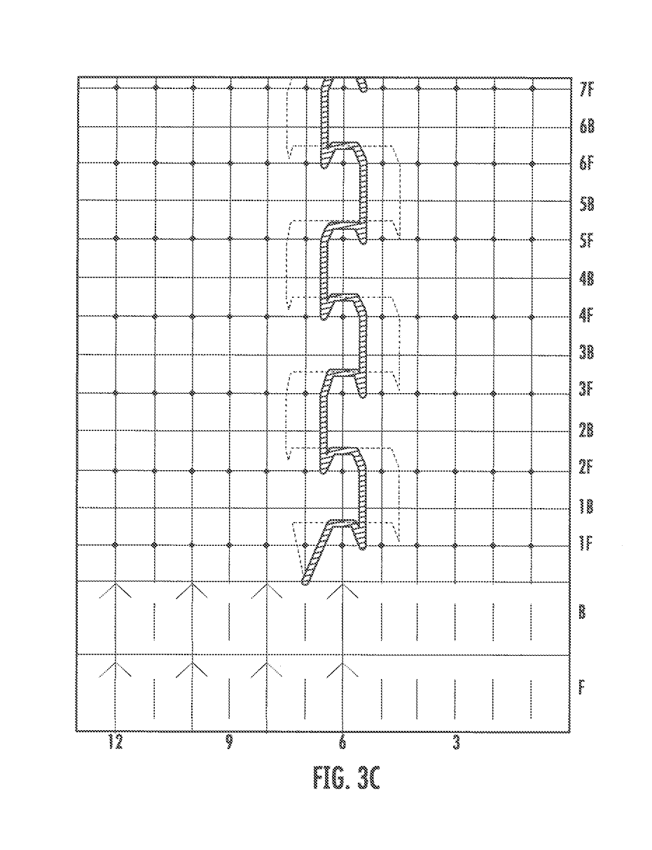

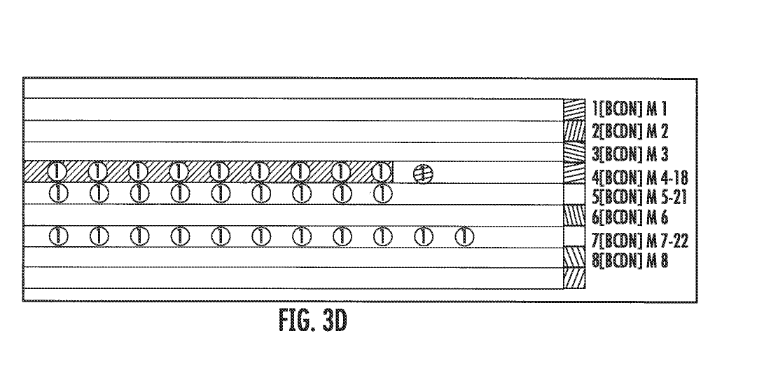

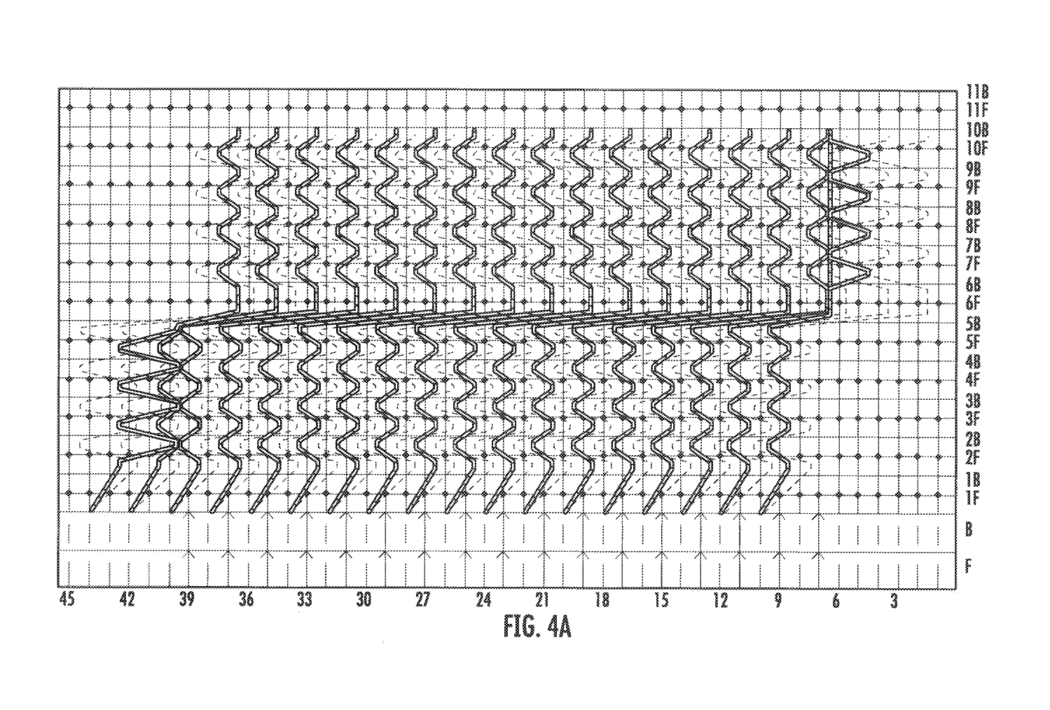

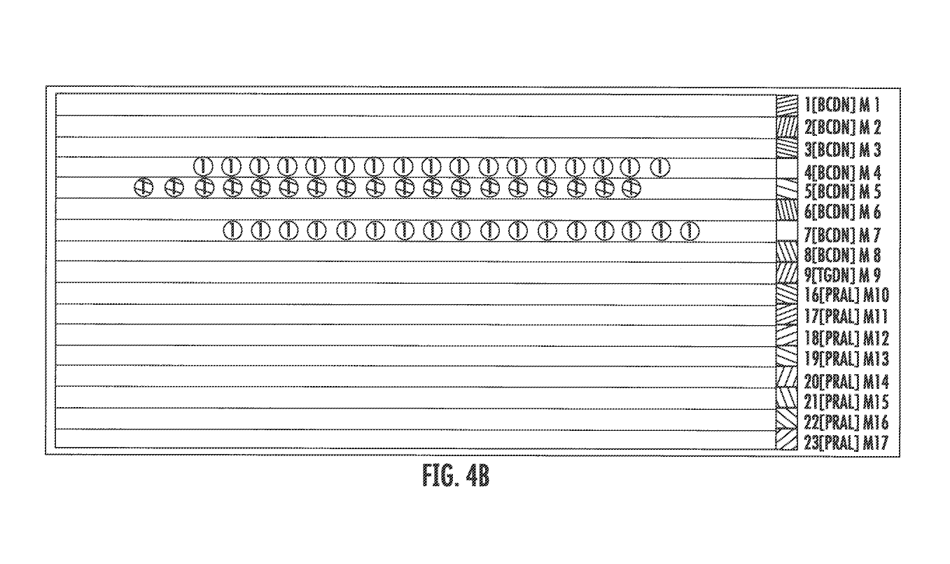

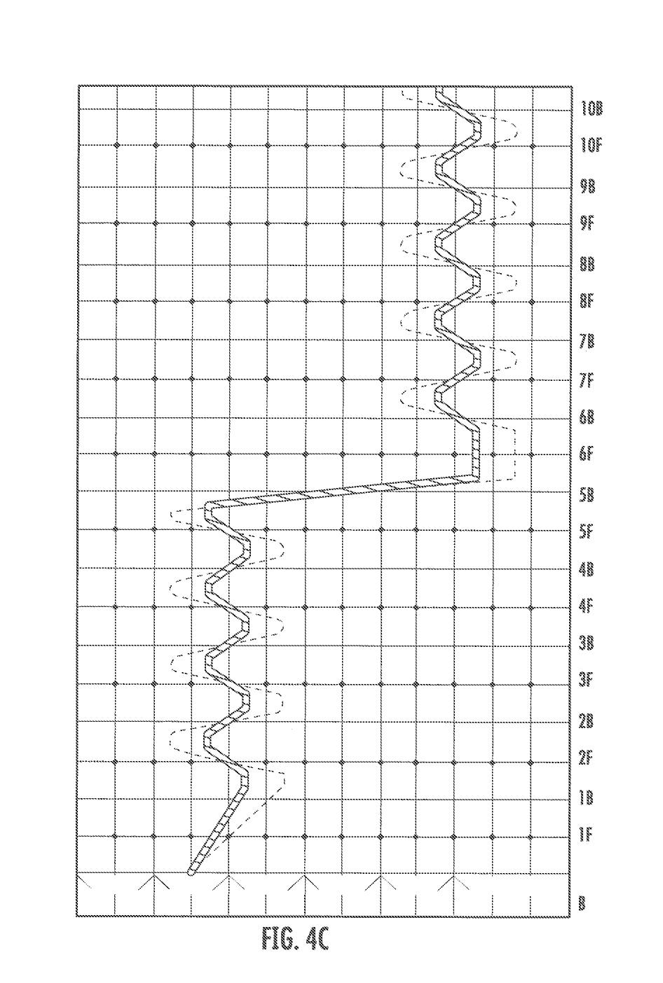

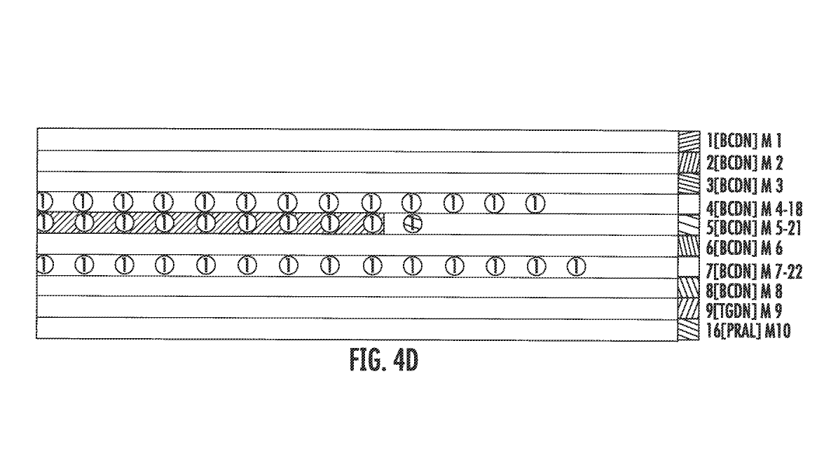

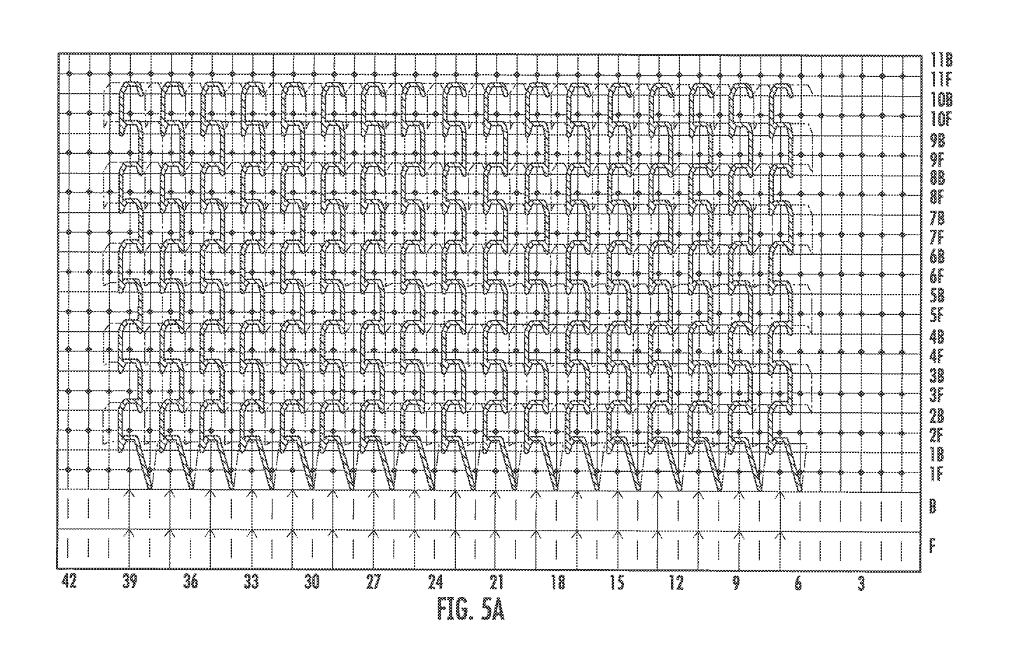

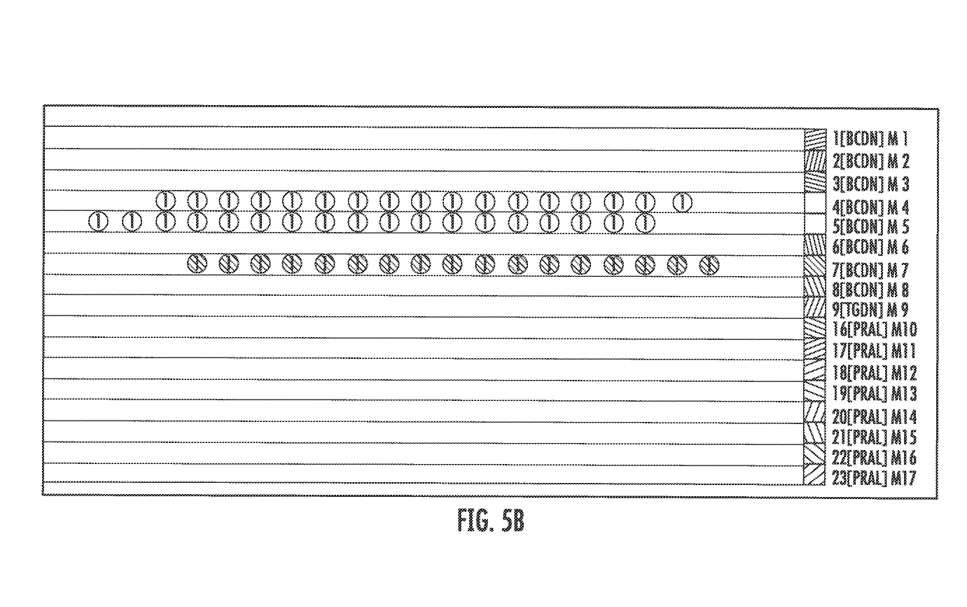

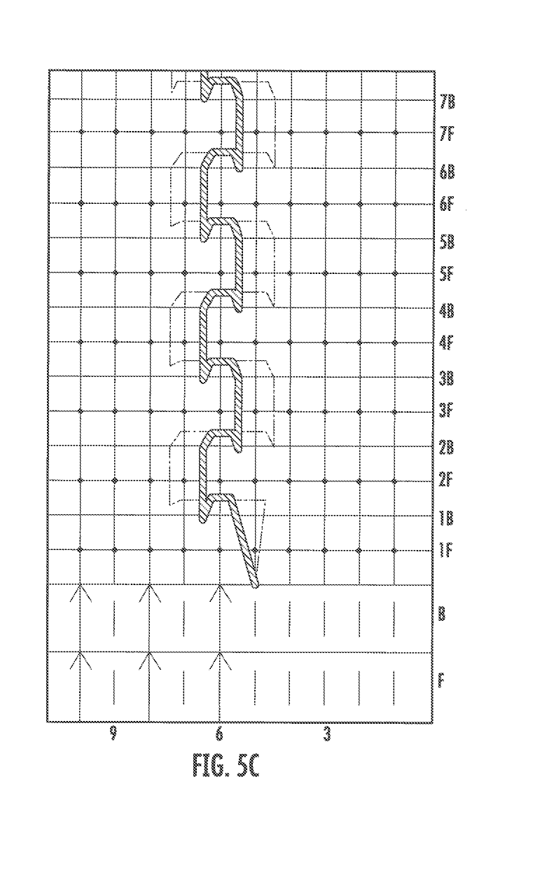

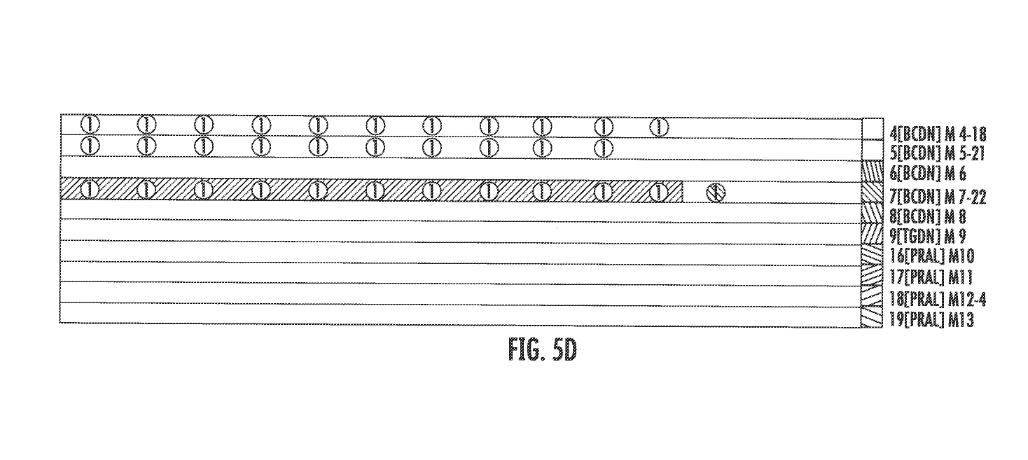

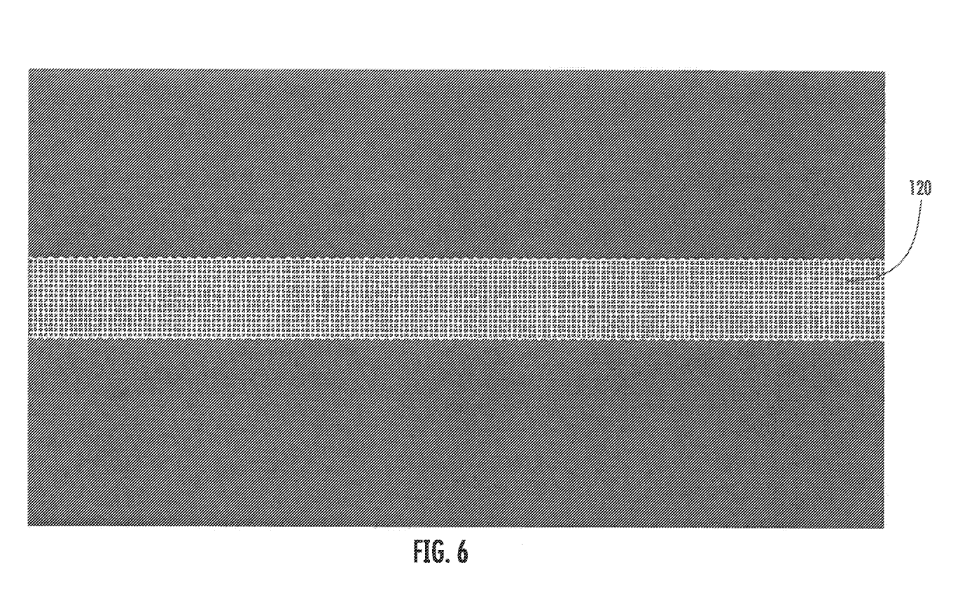

FIG. 2A illustrates a photograph of a pattern layout for a silk-based mesh or scaffold design in accordance with the present invention. In FIG. 2A item 100 is a mesh or scaffold. This variation of the scaffold in accordance with the present invention is preferably formed on a raschel knitting machine such as Comez DNB/EL-800-8B set up in 10 gg needle spacing by the use of three movements as shown in pattern layout in FIGS. 2B-2E: two movements in the wale direction, the vertical direction within the fabric, and one movement in the course direction, the horizontal direction of the fabric. The movements in the wale direction occur on separate needle beds with alternate yarns; loops that occur on every course are staggered within repeat. The yarn follows a repeat pattern of 3/1-1/1-1/3-3/3 for one of the wale direction movements (see ground bar #4) as shown in FIGS. 3A and 3B and FIGS. 3C and 3D and 1/1-1/3-3/3-3/1 for the other wale direction movement (see ground bar #7) as shown in FIGS. 5A and 5B, FIGS. 5C and 5D. The interlacing of the loops within the fabric allows for one yarn to become under more tension than the other under stress, locking it around the less tensioned yarn; keeping the fabric from unraveling when cut. Preferably, the fabric is a node-lock fabric as described in detail in U.S. patent application Ser. No. 12/680,404, the entirety of which is herein incorporated by reference. The other movement in the course direction as shown in FIGS. 4A-4D occurs in every few courses creating the porous design of the scaffold. These yarns follow a repeat pattern of 9/9-9/9-7/7-9/9-7/7-9/9-7/7-9/9/-1/1-1/1-3/3-1/1-3/3-1/1-3/3-1/1-3/3-1/1 (see ground bar #5) for the course direction movement as shown in FIGS. 4A and 4B and FIGS. 4C and 4D. The pattern simulation layout of this pattern is rendered with ComezDraw 3 software in FIG. 6 considering a yarn design made with 2 ends of Td (denier count) 20/22 raw silk twisted together in the S direction to form a ply with 6 tpi (turns per inch) and further combining three of the resulting ply with 3 tpi. The same yarn design is used for the movements occurring in the wale and course directions. The stitch density or pick count for the scaffold in FIG. 6 is 40 picks per centimeter considering the total picks count for the technical front face and the technical back face of the fabric, or 20 picks per cm considering only on the face of the fabric. In FIG. 6 item 120 is a simulated double needle bed mesh or scaffold. The operating parameters are not limited to those described in FIGS. 2B-2E, but are merely the optimum values for the specific yarn design used for the pattern simulation layout of FIG. 6.

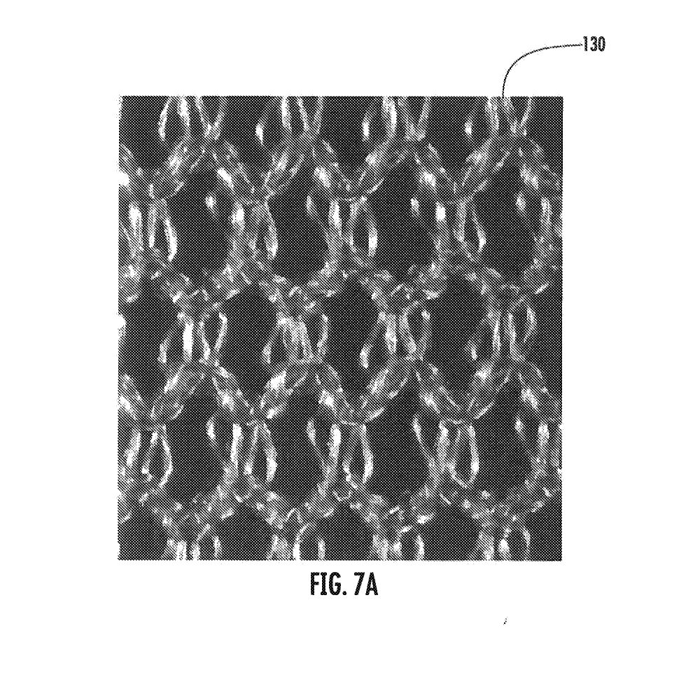

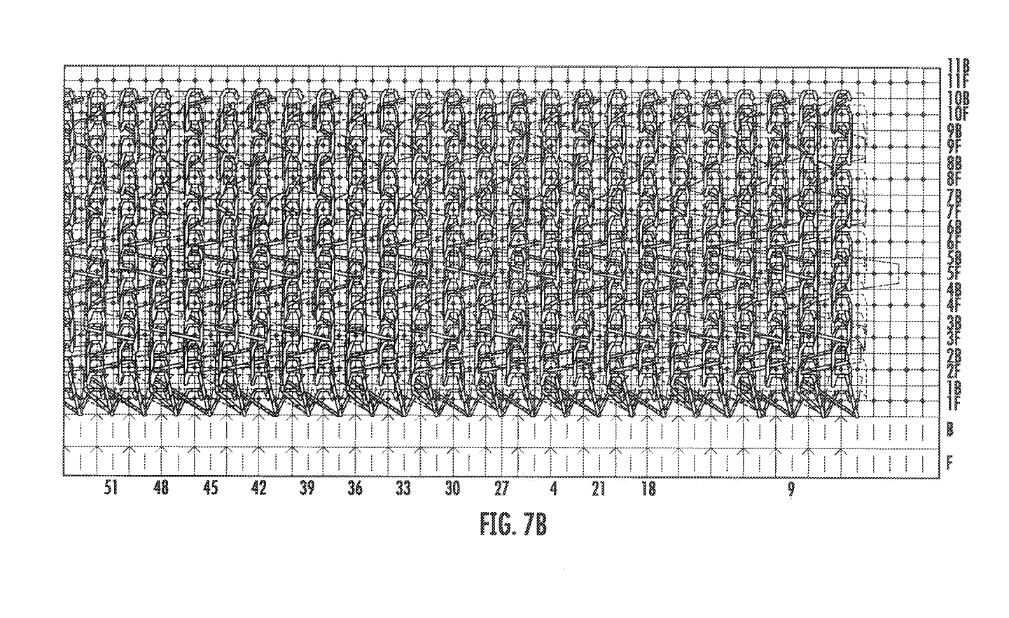

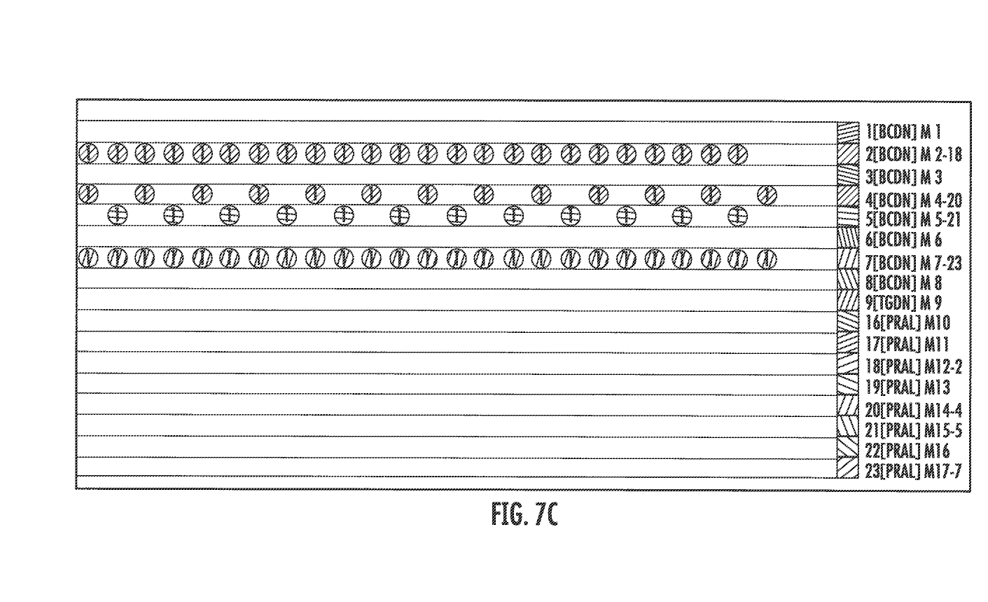

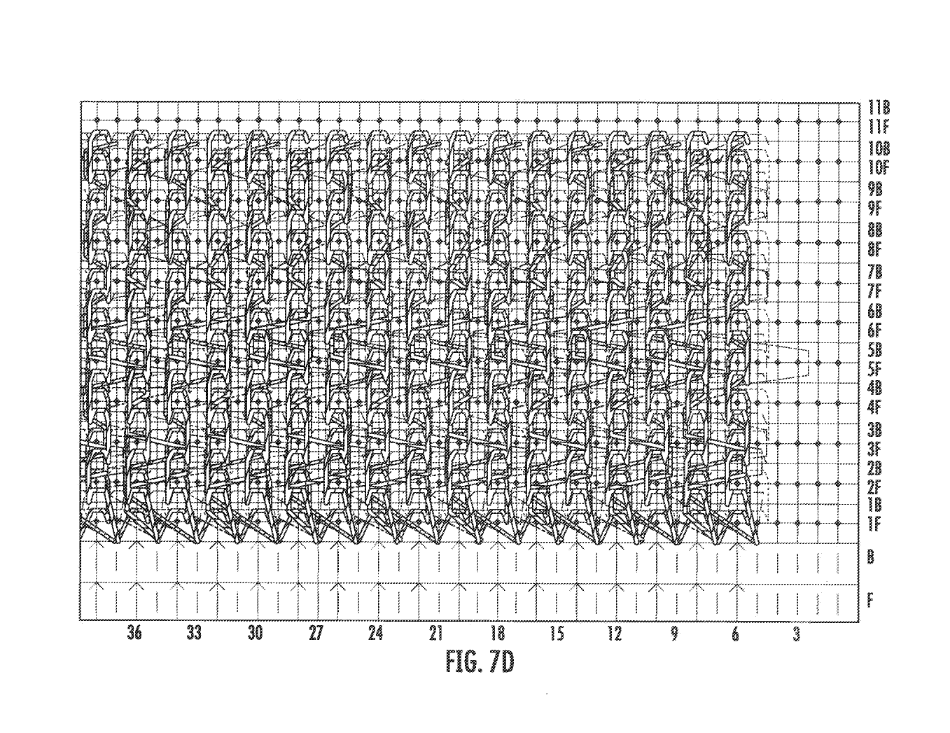

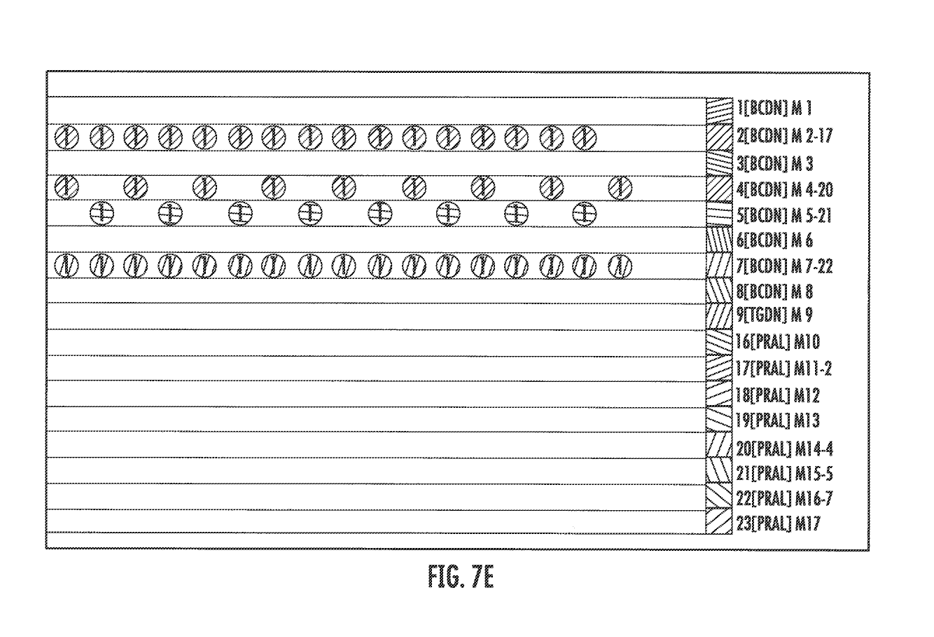

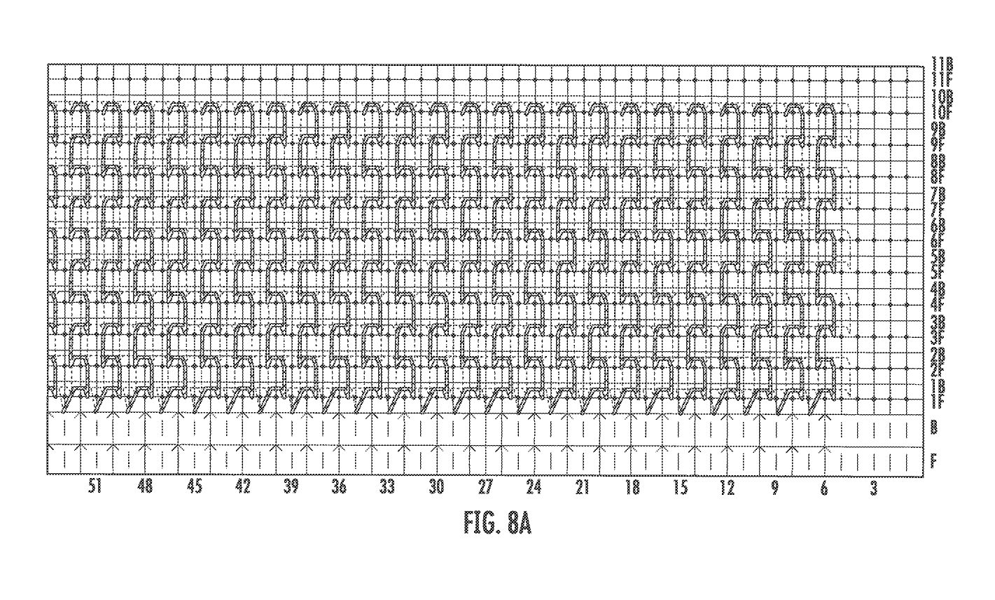

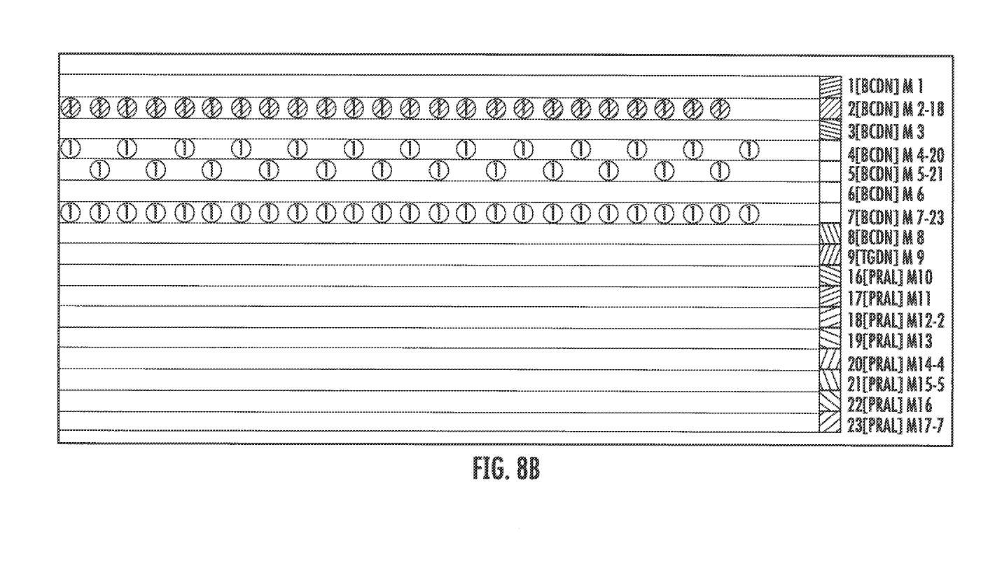

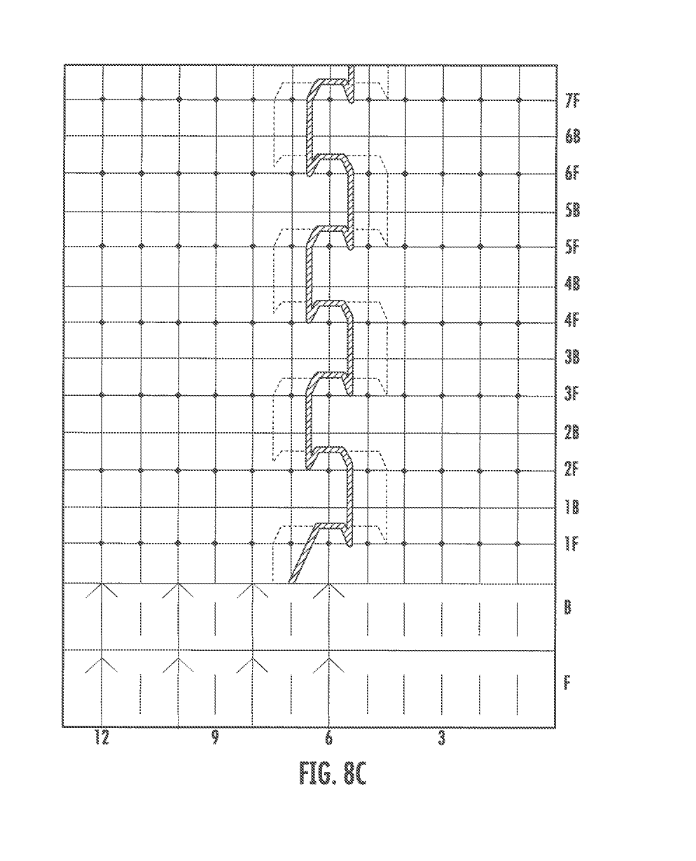

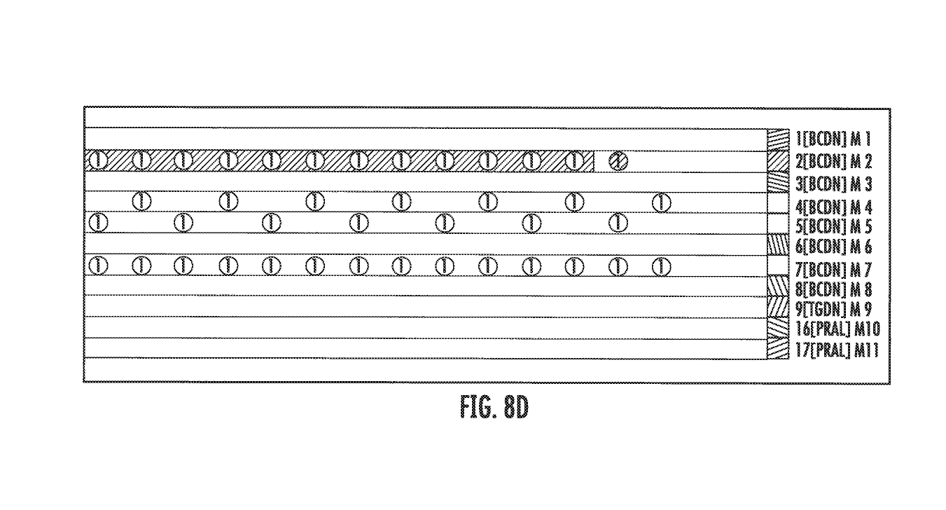

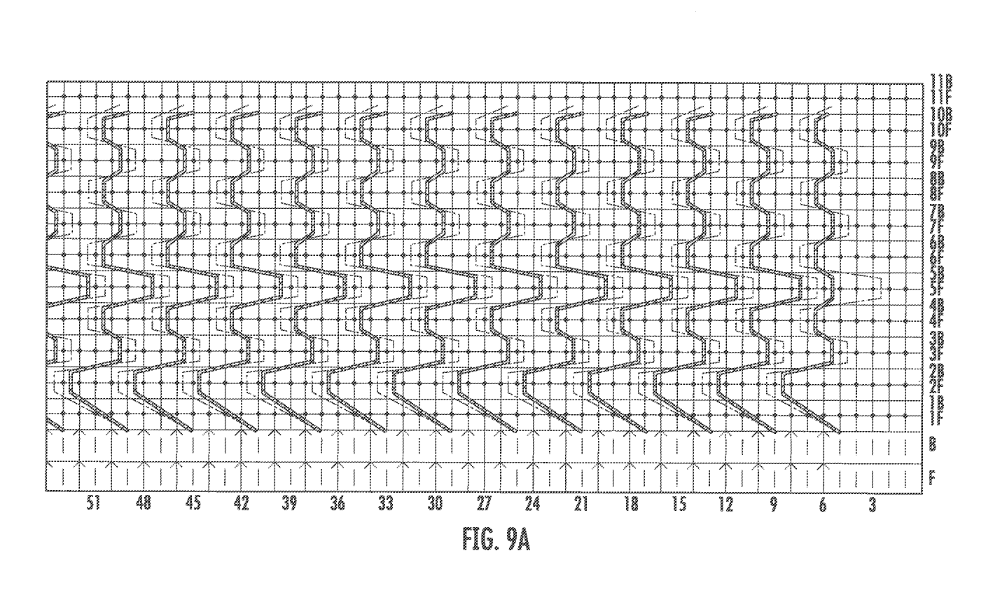

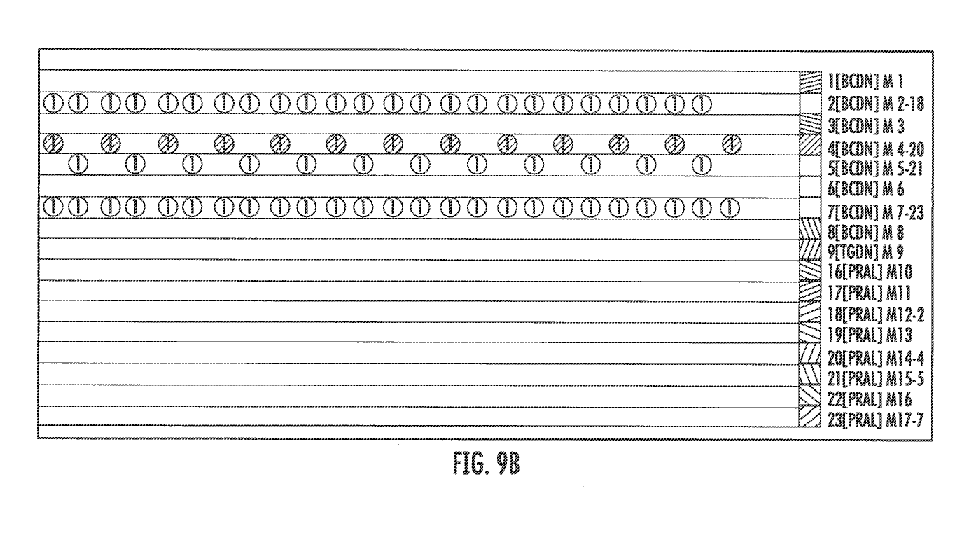

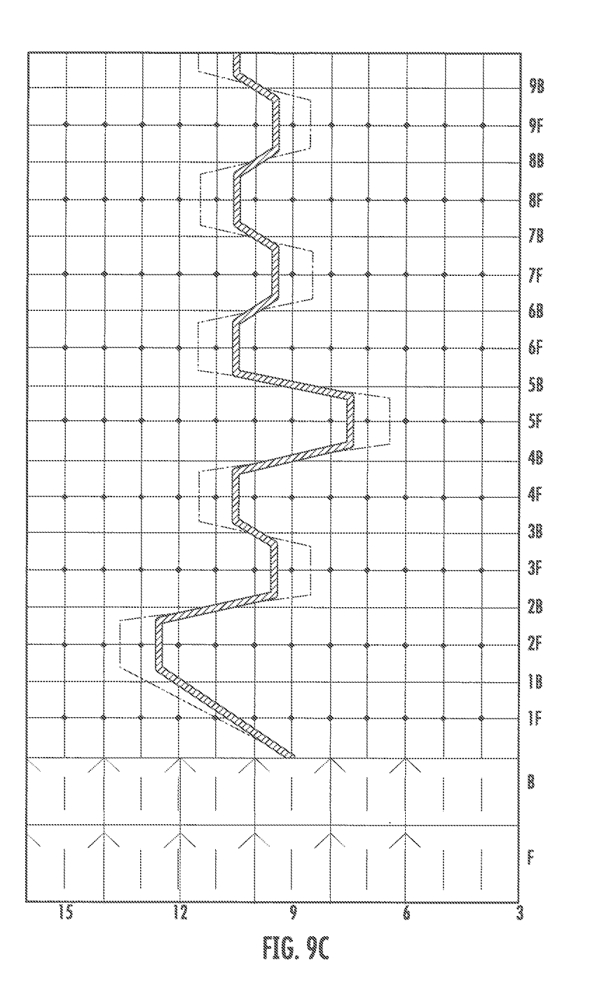

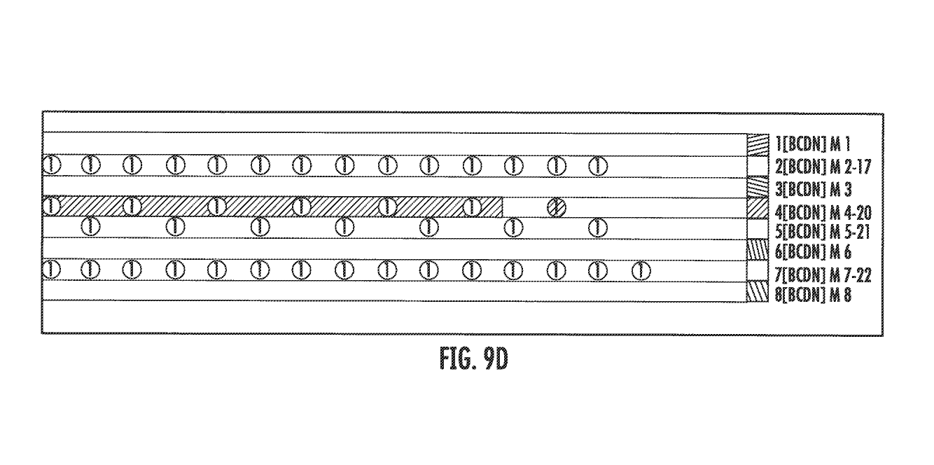

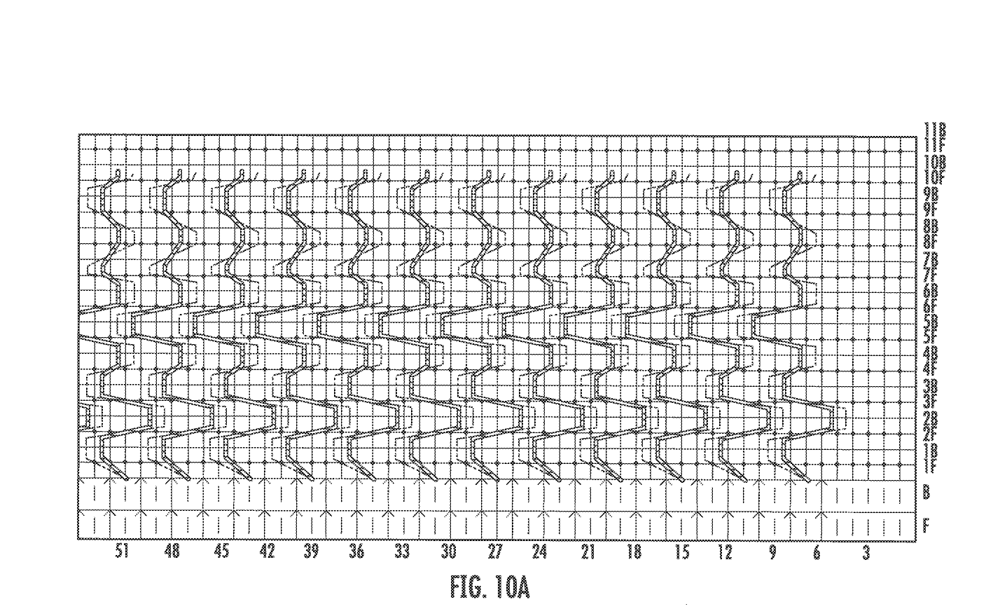

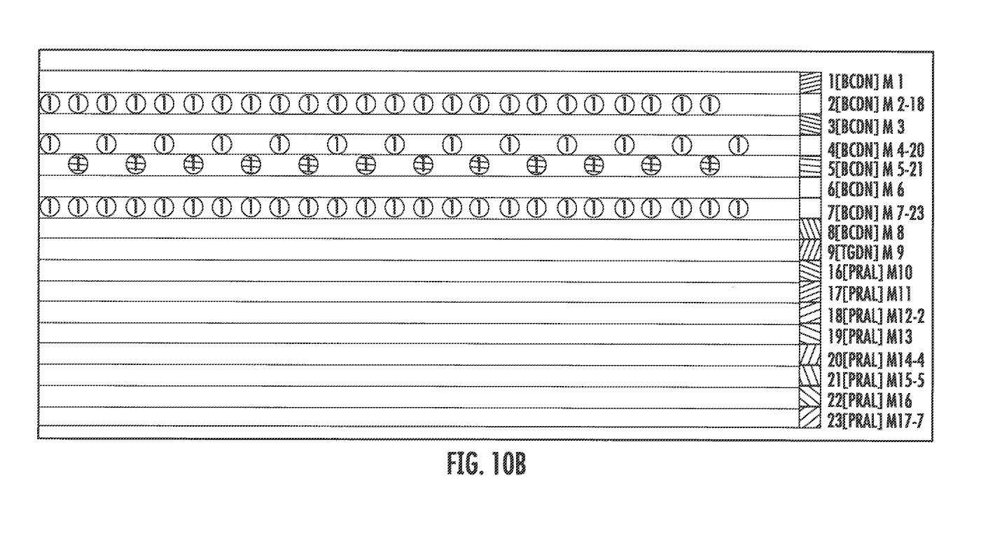

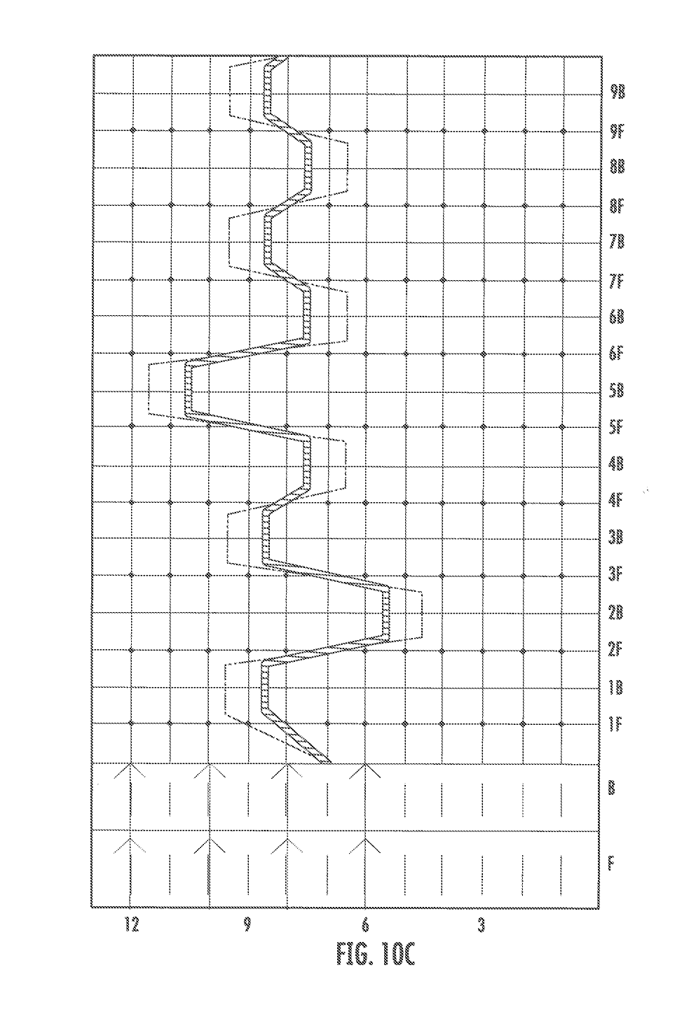

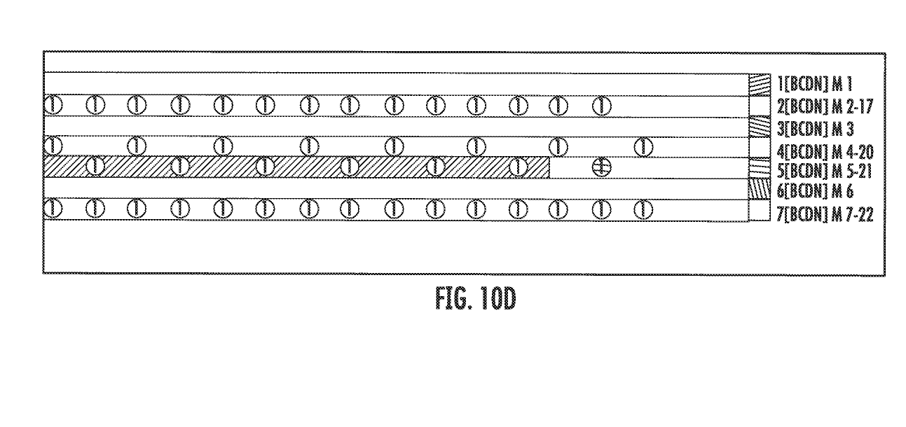

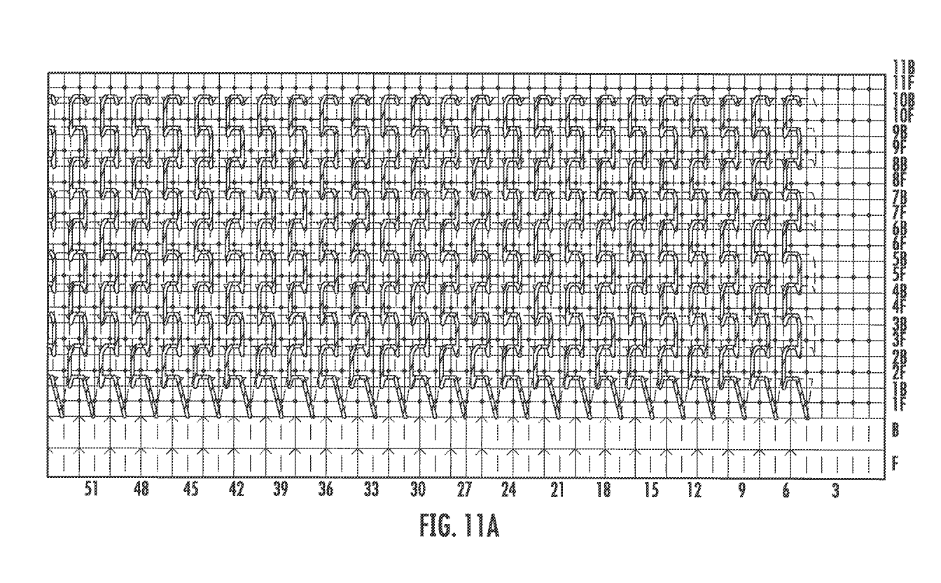

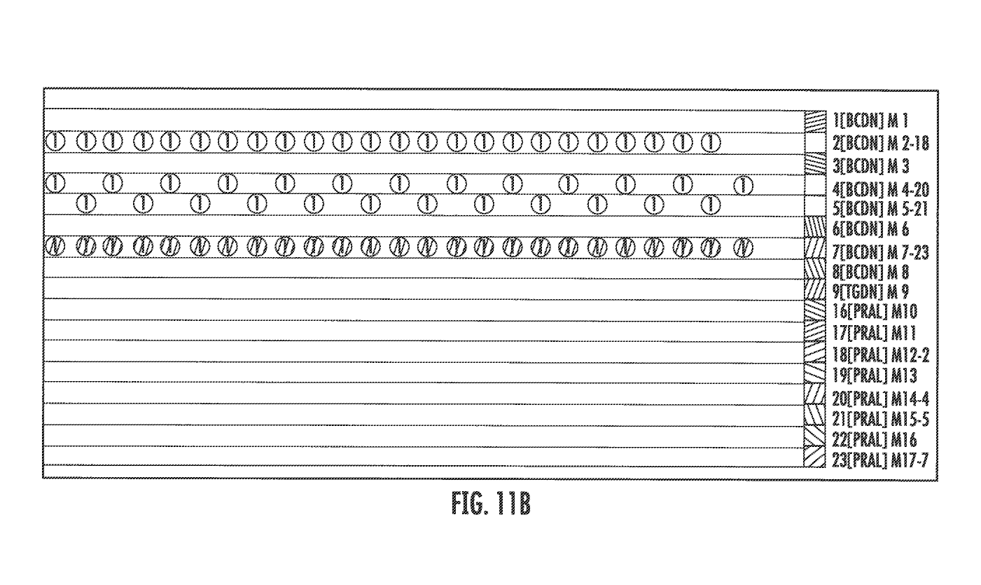

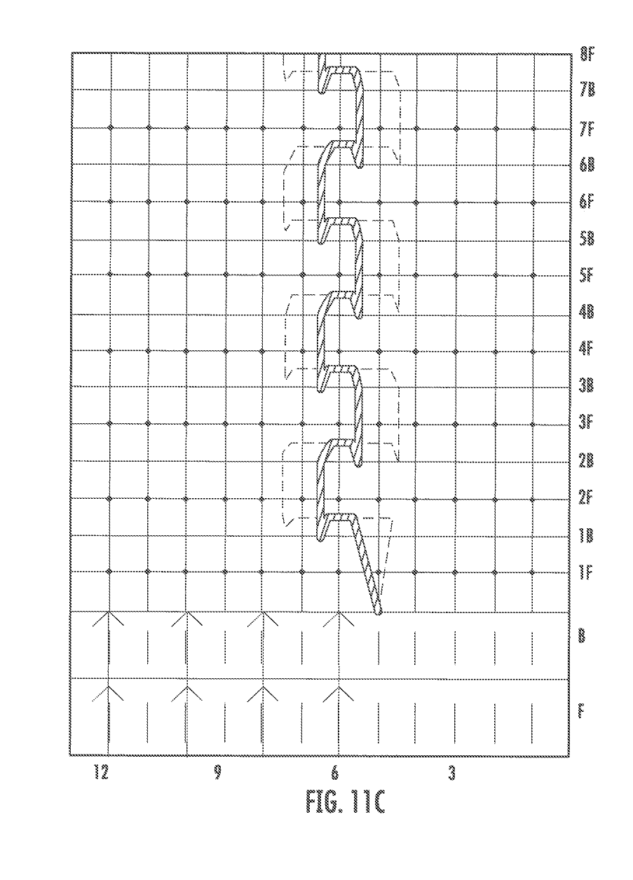

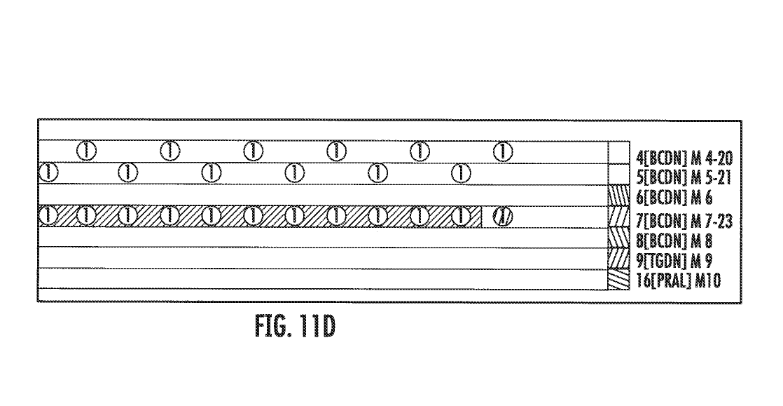

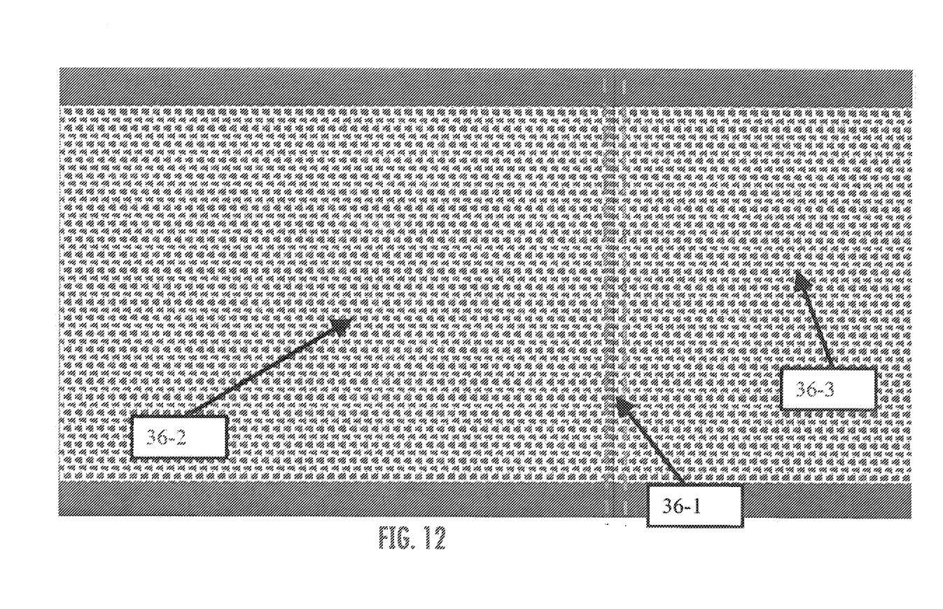

FIG. 7A is a photograph of a pattern layout for a silk-based scaffold design in accordance with the present invention. In FIG. 7A item 130 is a mesh or scaffold. This variation of the scaffold in accordance with the present invention is preferably created on a raschel knitting machine such as Comez DNB/EL-800-8B set up in 10 gg needle spacing by the use of four movements as shown in pattern layout in FIG. 7B and 7C and FIGS. 7D and 7E: two movements in the wale direction, the vertical direction within the fabric, and two movements in the course direction, the horizontal direction of the fabric. The movements in the wale direction occur on separate needle beds with alternate yarns; loops that occur on every course are staggered within repeat. The yarn follows a repeat pattern of 3/1-1/1-1/3-3/3 for one of the wale direction movements as shown in FIGS. 8A-8D and 1/1-1/3-3/3-3/1 for the other wale direction movement as shown in FIGS. 11A-11D. The interlacing of the loops within the fabric allows for one yarn to be under more tension than the other under stress, locking it around the less tensioned yarn; keeping the fabric from unraveling when cut. One of the other two movements in the course direction as shown in FIGS. 9A-D occurs in every few courses creating the porous design of the scaffold. These yarns follow a repeat pattern of 3/3-3/3-7/7-7/7-3/3-3/3-5/5-5/5-1/1-1/1-5/5-5/5-3/3-3/3-5/5-5/5-3/3-3/3-5/5-5/5 for the course direction movement. The other movements in the course direction as shown in FIGS. 10A-D occur in every few courses creating the openings in the scaffold. These yarns follow a repeat pattern of 3/3-3/3-5/5-5/5-1/1-1/1-5/5-5/5-3/3-3/3-7/7-7/7-3/3-3/3-5/5-5/5-3/3-3/3-5/5-5/5-3/3 for the course direction movement. The pattern simulation layout of this pattern is rendered with ComezDraw 3 software in FIG. 12 considering a yarn design made with 2 ends of Td 20/22 raw silk twisted together in the S direction to form a ply with 6 tpi and further combining three of the resulting ply with 3 tpi. The same yarn design is used for the movements occurring in the wale and course directions. The stitch density or pick count for the scaffold design in FIG. 12 is 39 picks per centimeter considering the total picks count for the technical front face and the technical back face of the fabric, or 19.5 picks per cm considering only one face of the fabric. The operating parameters are not limited to those described in FIGS. 7B-E, but just the optimum values for the specific yarn design used for the pattern simulation layout of FIG. 12.

Furthermore, FIG. 12 demonstrates a process improvement for the manufacturing process of the scaffold with the pattern layout in FIG. 7B-E. The improvement consists of a separation area, 36-1, between two individual scaffolds, 36-2 and 36-3. The advantage of the separation area is to provide guidance for the correct length that the scaffold needs to measure and to provide guidance for the tools necessary for separating two individual scaffolds. For example in order to achieve a length of 5 cm±0.4 cm, the pattern in FIGS. 7B-E requires repeating from pattern line 1 to pattern line 16 for 112 times followed by a repeat of 2 times from pattern line 17 to pattern line 20.

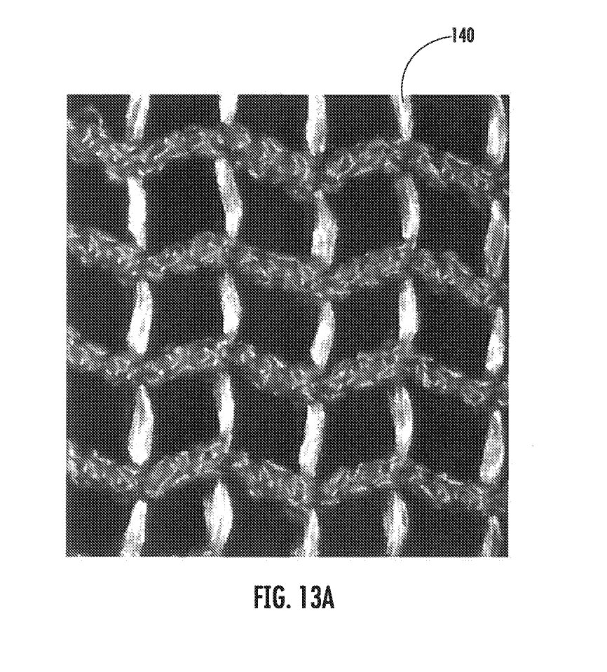

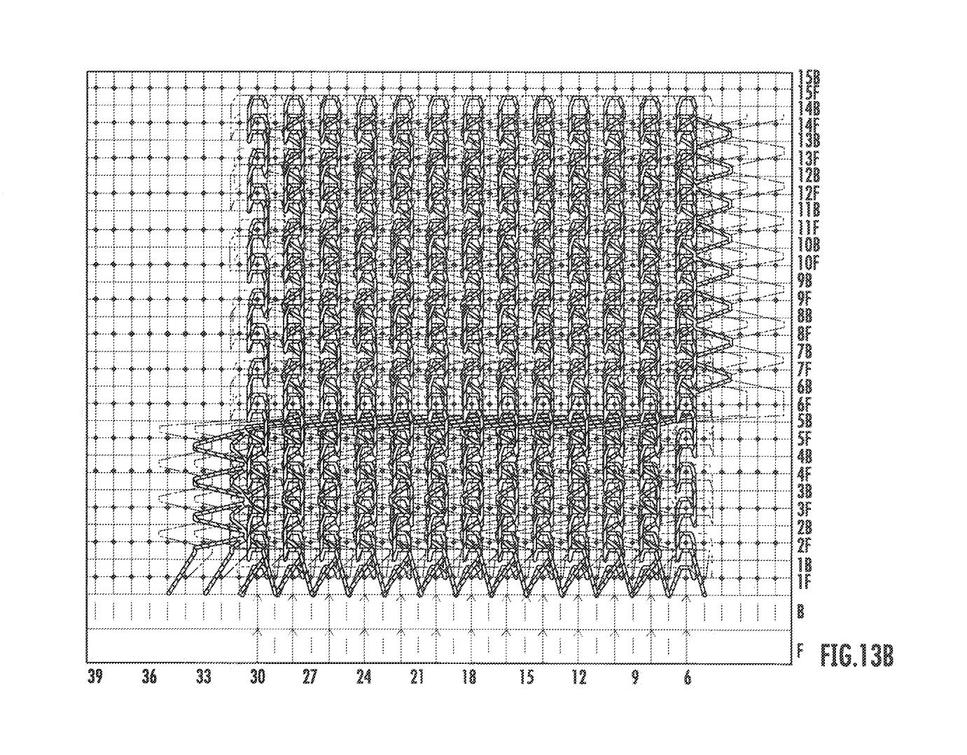

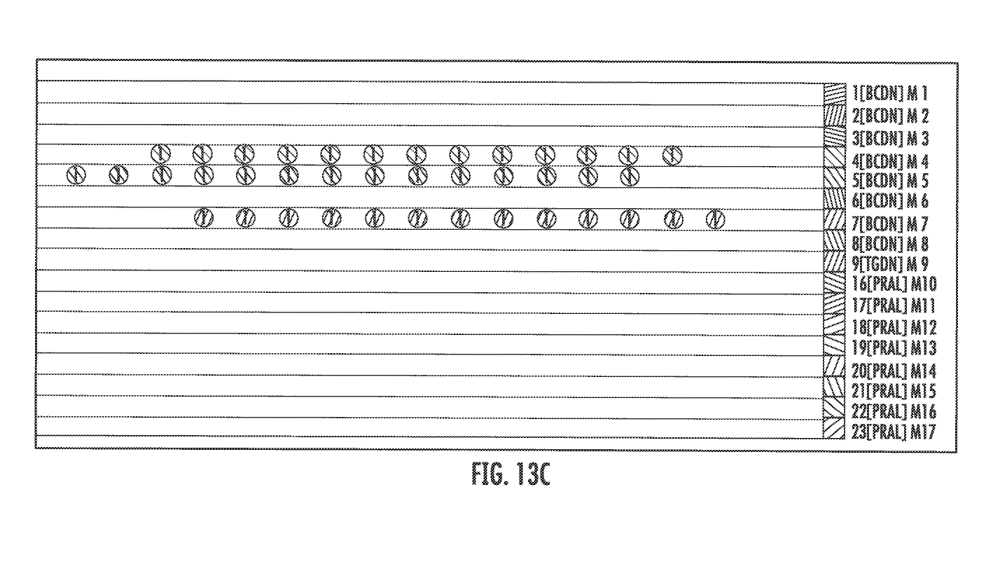

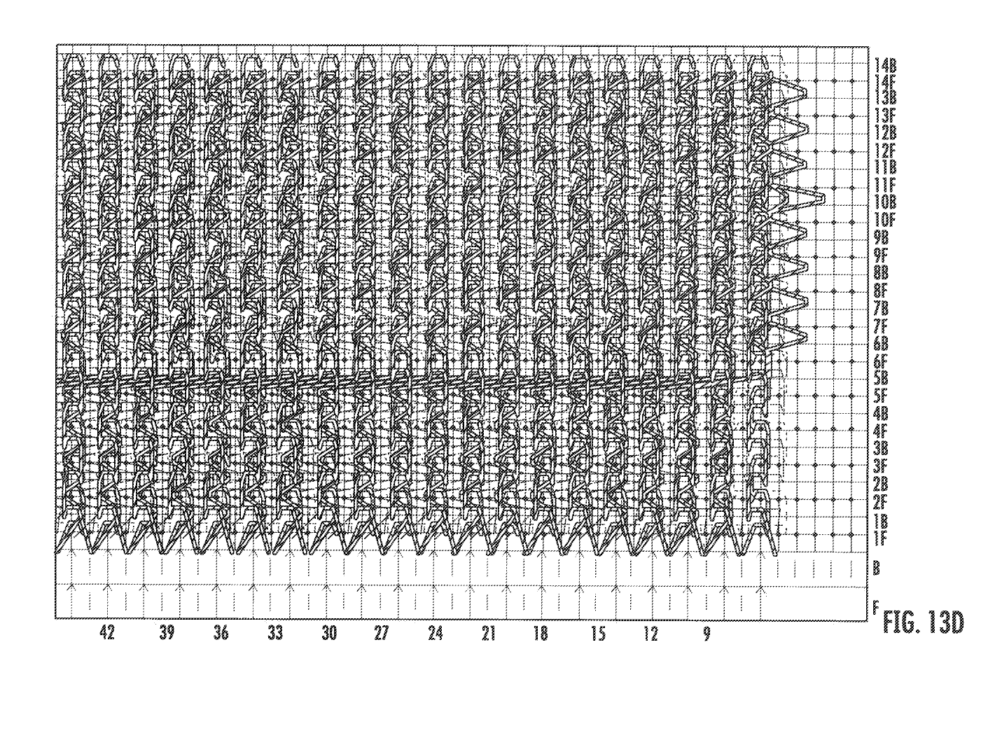

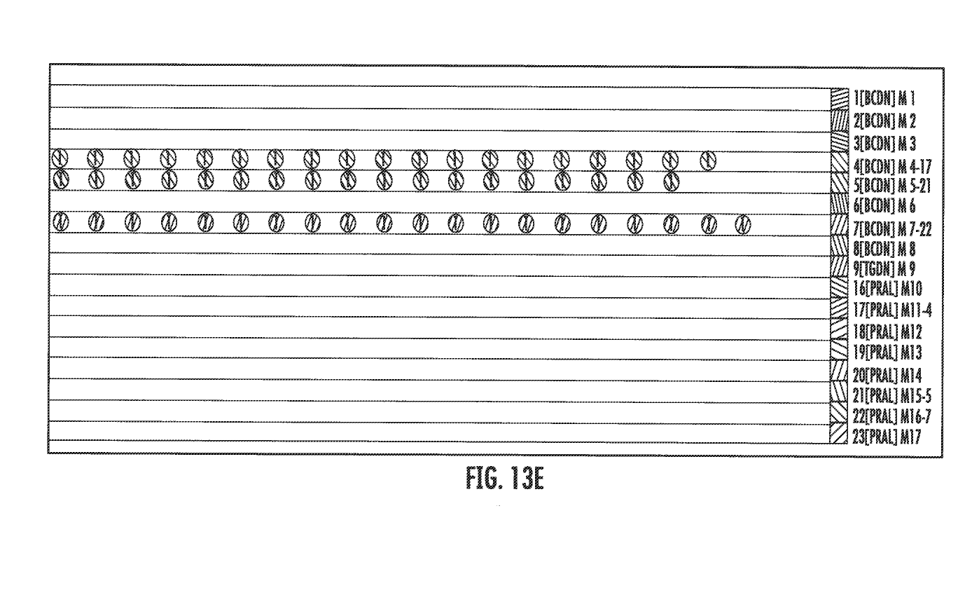

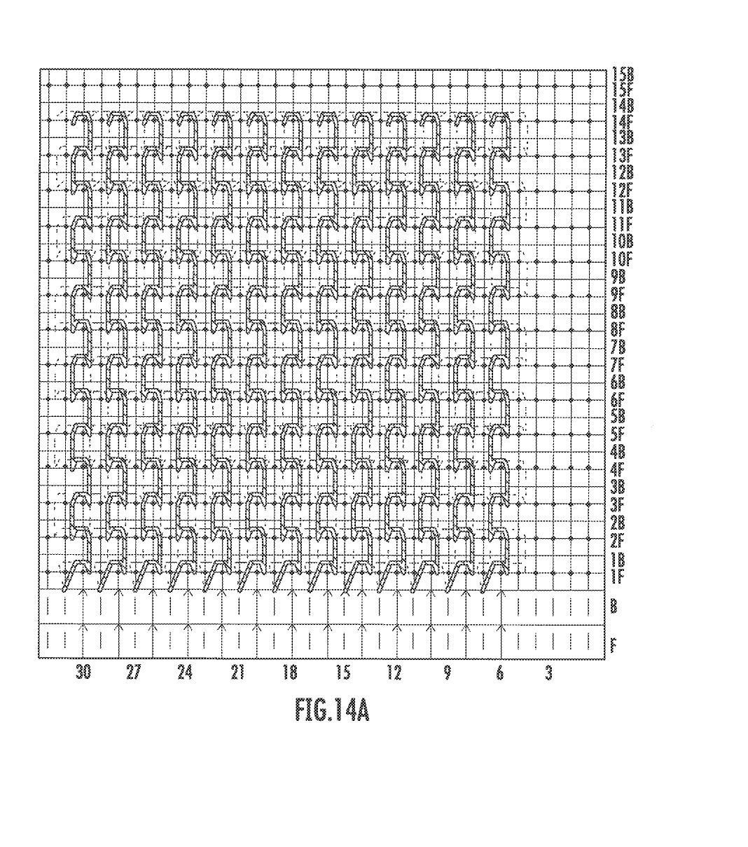

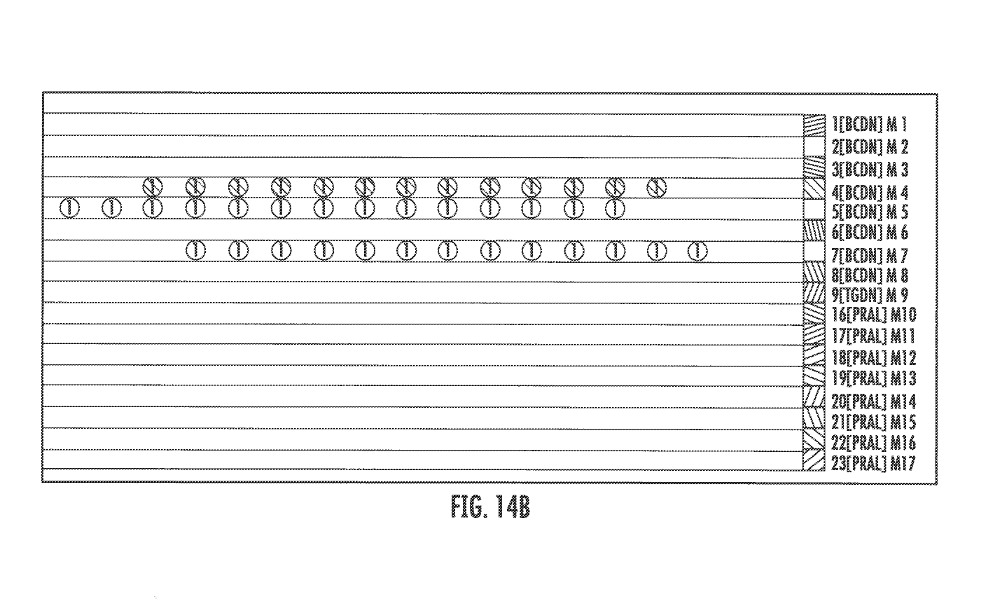

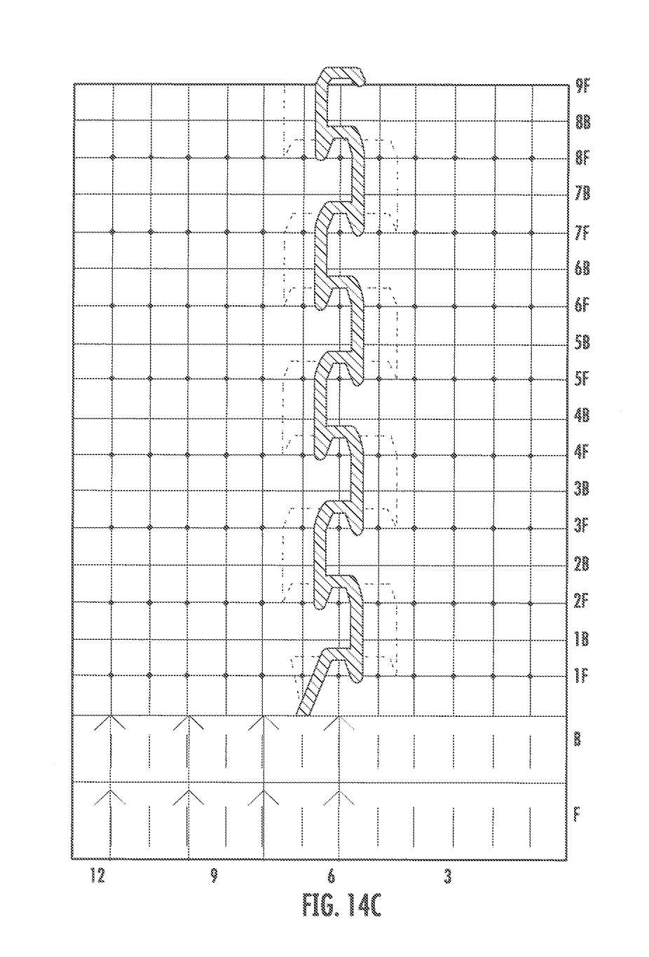

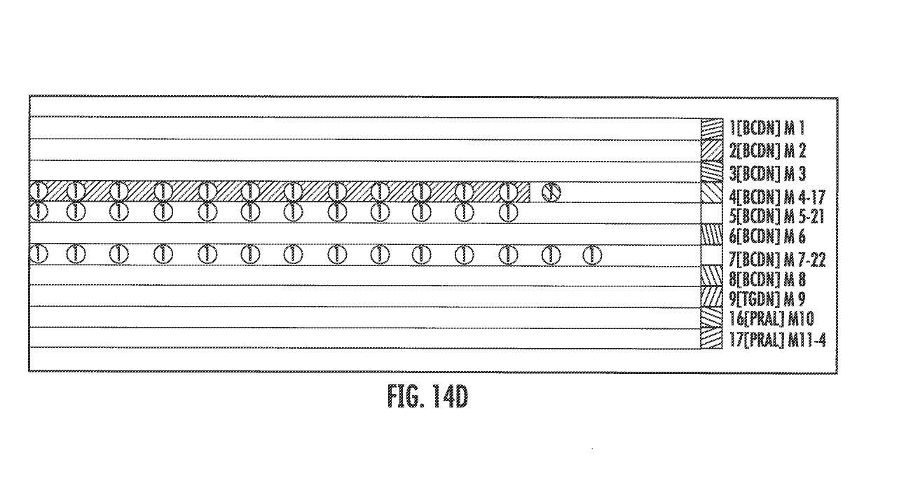

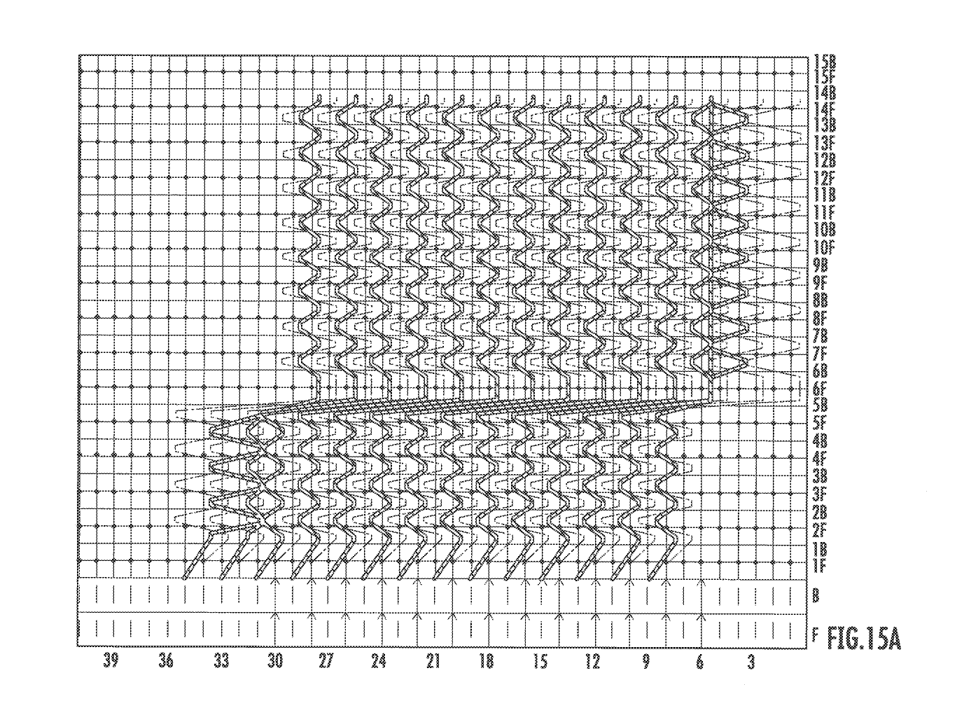

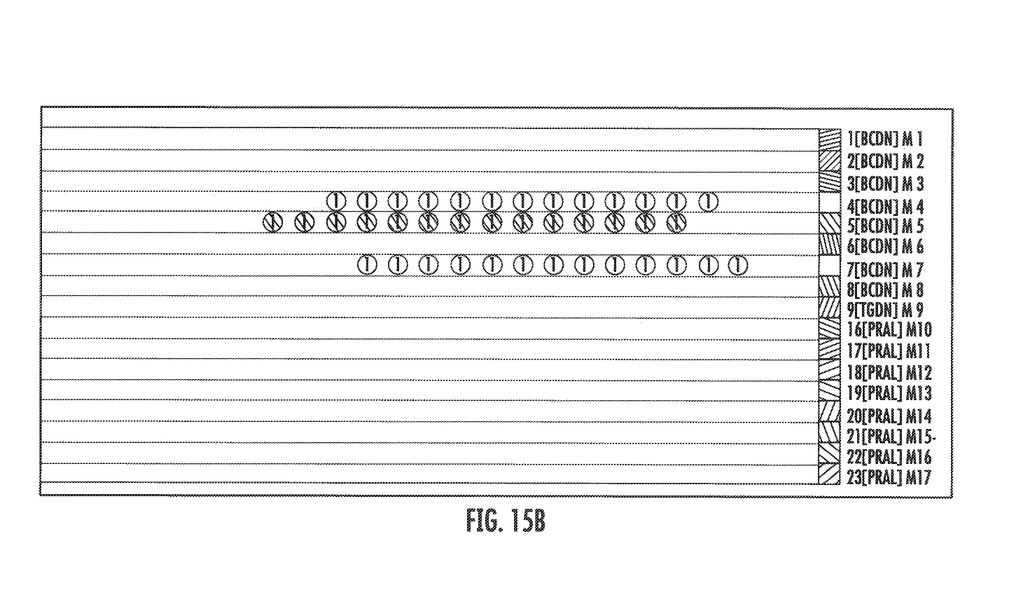

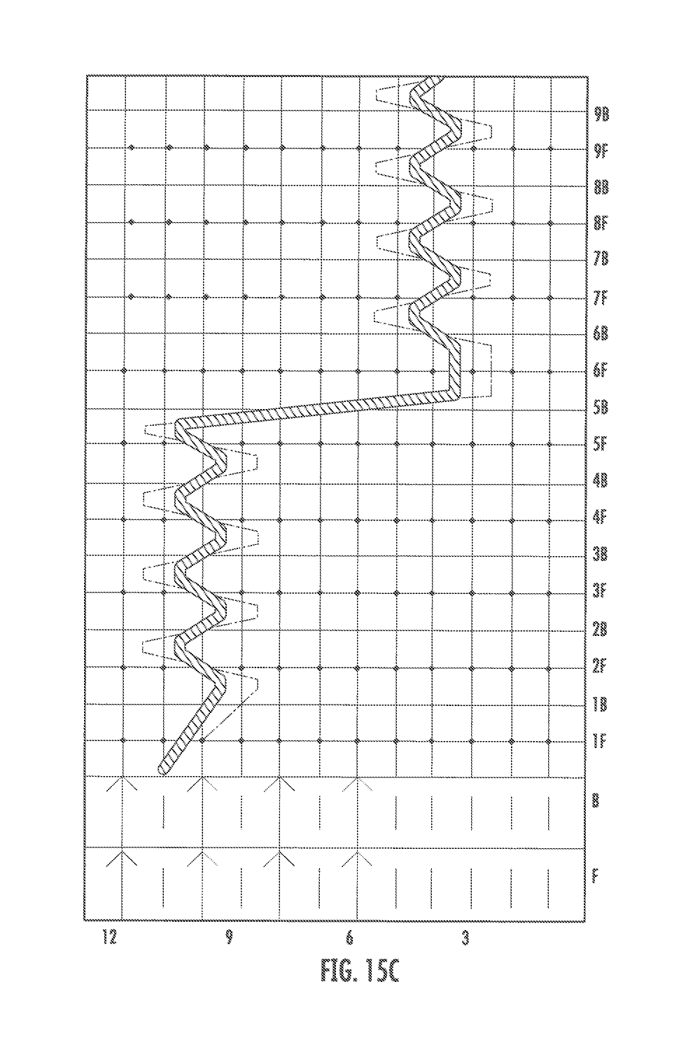

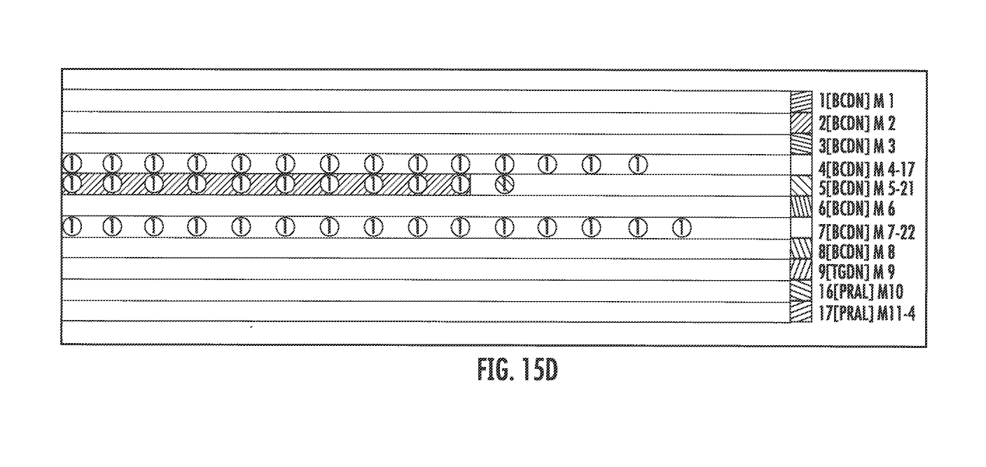

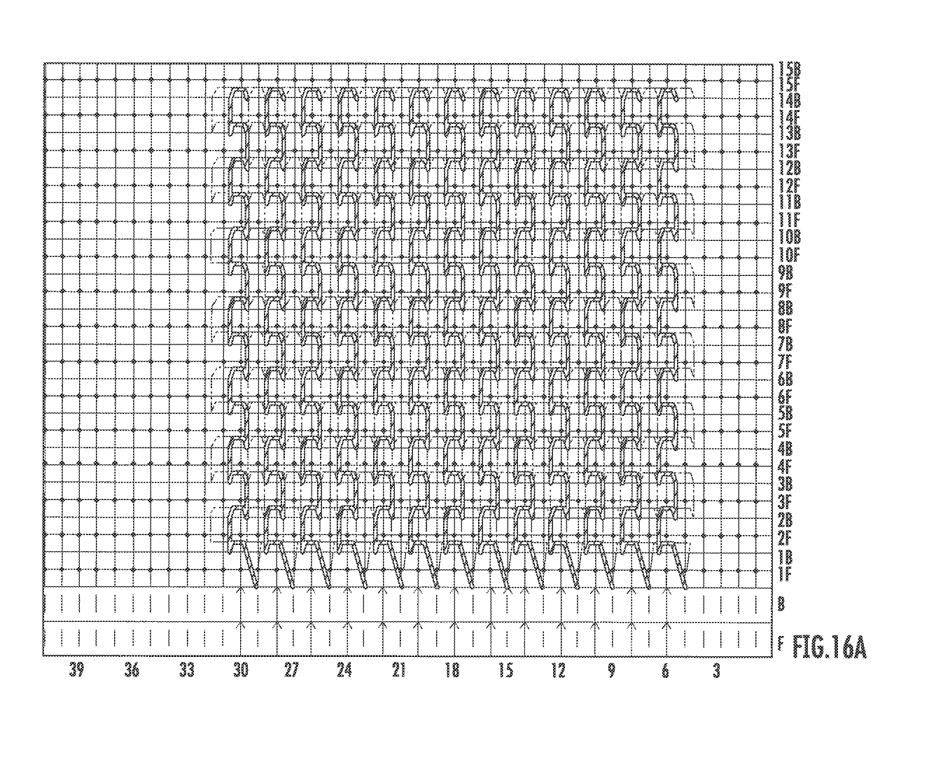

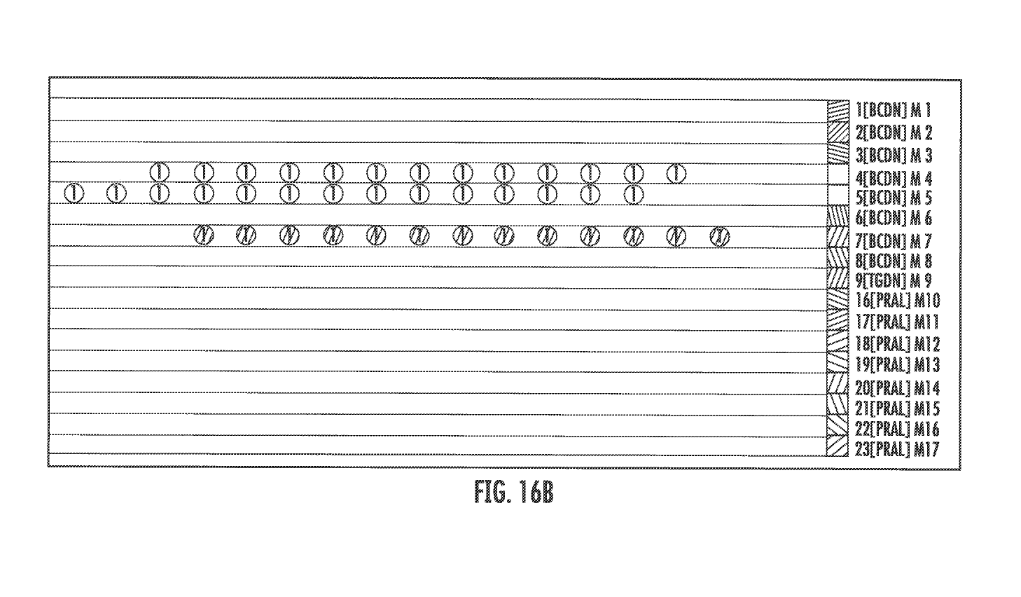

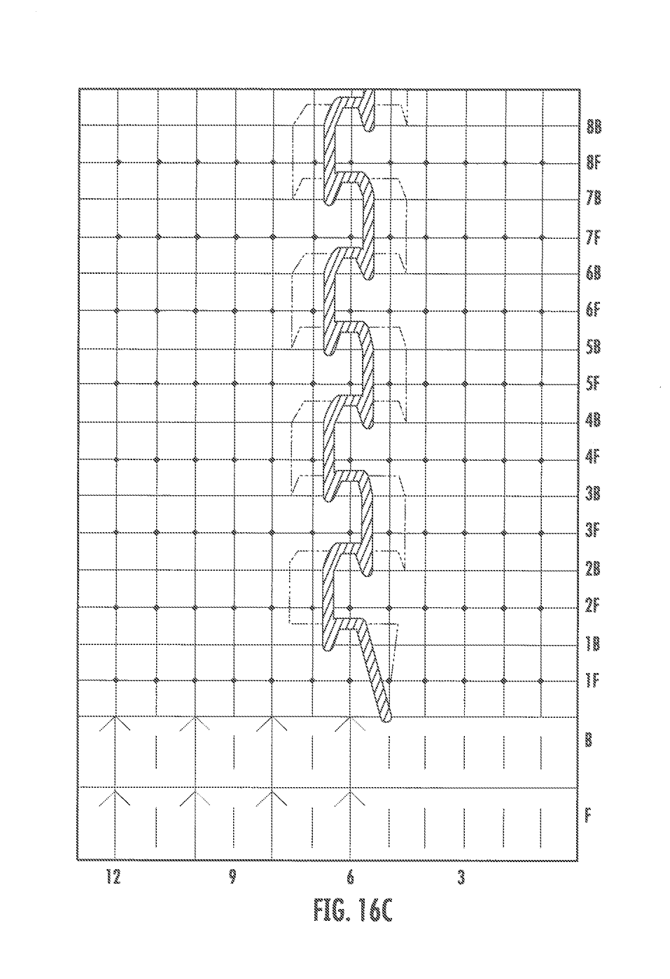

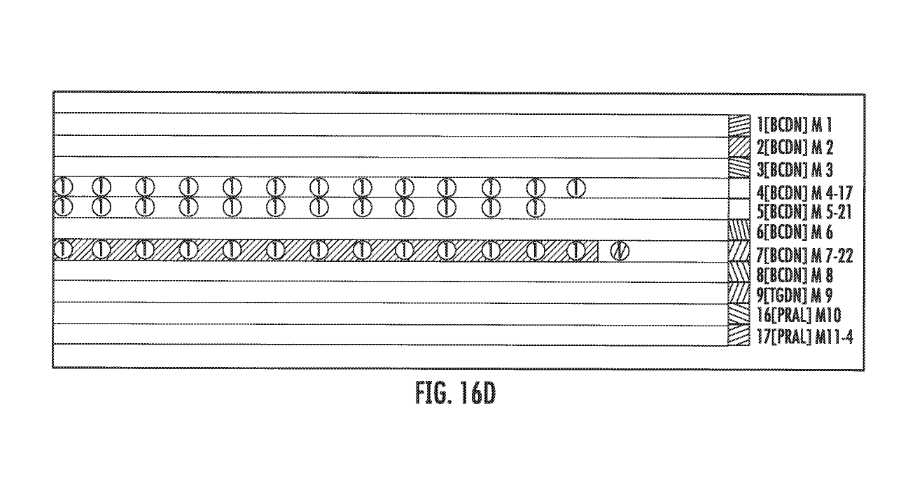

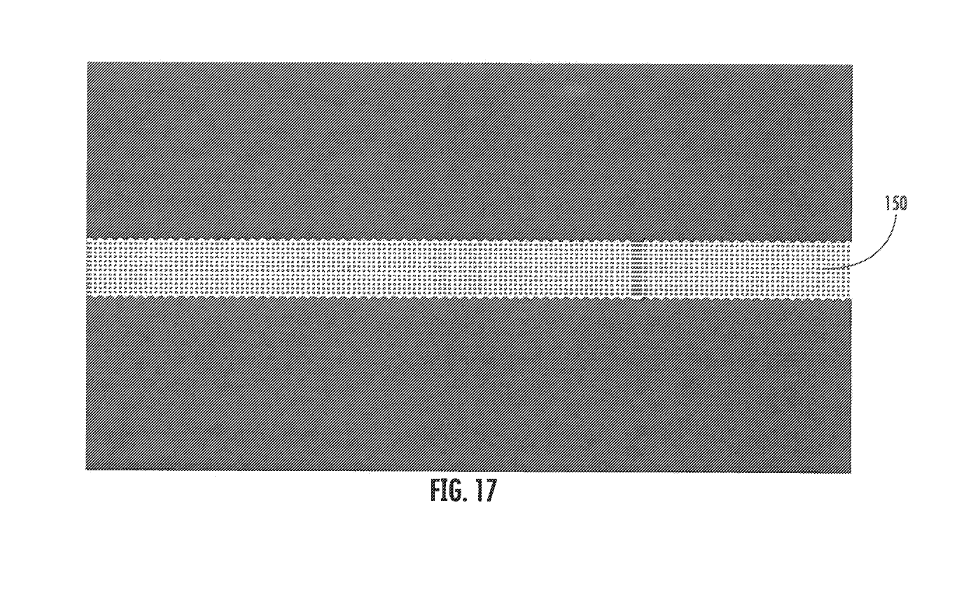

FIG. 13A is a photograph of a pattern layout for a silk-based scaffold design in accordance with the present invention. In FIG. 13A item 140 is a mesh or scaffold. This variation of the scaffold according to an aspect of the present invention is preferably created on a raschel knitting machine such as Comez DNB/EL-800-8B set up in 10 gg needle spacing by the use of three movements as shown in pattern layout in FIGS. 13B-E: two movements in the wale direction, the vertical direction within the fabric, and one movement in the course direction, the horizontal direction of the fabric. The movements in the wale direction occurs on separate needle beds with alternate yarns; loops that occur on every course are staggered within repeat. The yarn follows a repeat pattern of 3/1-1/1-1/3-3/3-for one of the wale direction movements shown in FIGS. 14A-D and 1/1-1/3-3/3-3/1 for the other wale direction movement as shown in FIGS. 16A-D. The interlacing of the loops within the fabric allows for one yarn to be under more tension than the other under stress, locking it around the less tensioned yarn; keeping the fabric from unraveling when cut. The other movement in the course direction which is shown in FIG. 15A-D occurs in every few courses creating the porous design of the scaffold. These yarns follow a repeat pattern of 9/9-9/9-7/7-9/9-7/7-9/9-7/7-9/9-7/7-9/9-11-11/1-3/3-1/1-3/3-1/1-3/3-1/1-3/3-1/1 for the course direction movement. The pattern simulation layout of this pattern is rendered with ComezDraw 3 software in FIG. 17 considering a yarn design made with 3 ends of Td 20/22 raw silk twisted together in the S direction to form a ply with 6 tpi and further combining three of the resulting ply with 3 tpi. The same yarn design is used for the movements occurring in the wale and course directions The stitch density or pick count for the design in FIG. 17 is 34 picks per centimeter considering the total pick count for the technical front face and the technical back face of the fabric, or 17 picks per cm considering only on the face of the fabric. The operating parameters are not limited to those described in FIGS. 13B-E, but just the optimum values for the specific yarn design used for the pattern simulation layout of FIG. 17. In FIG. 17 item 150 is a simulated double needle bed scaffold.

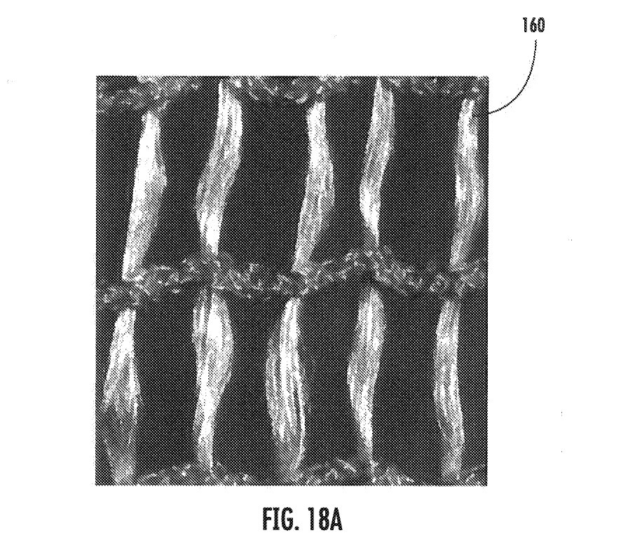

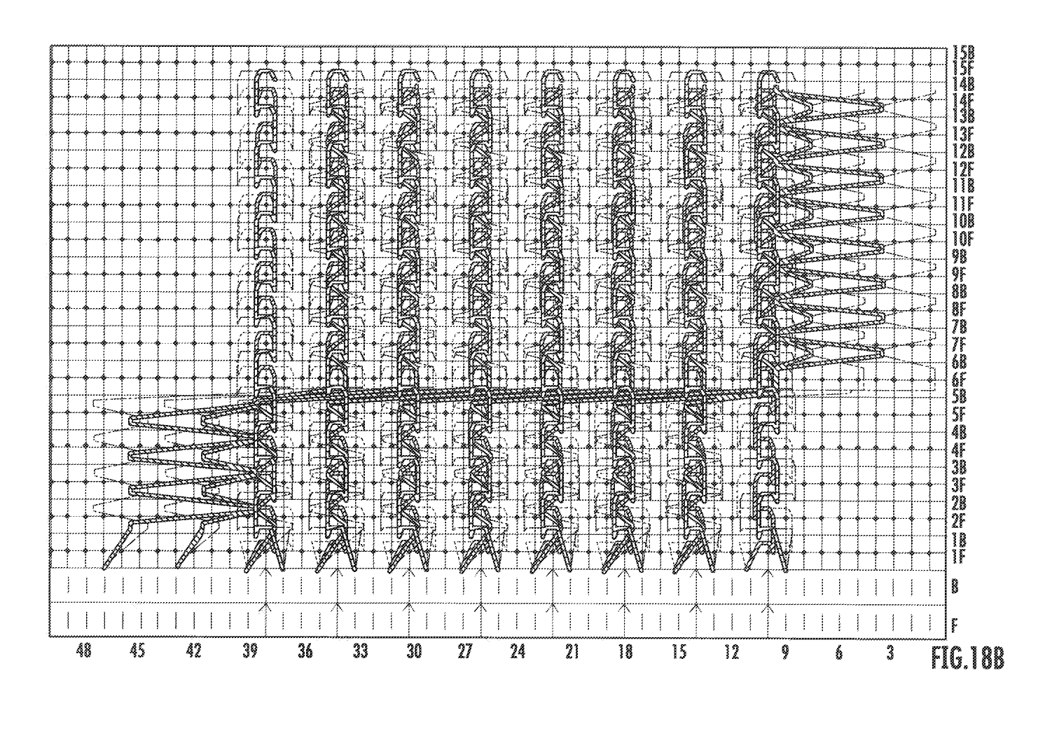

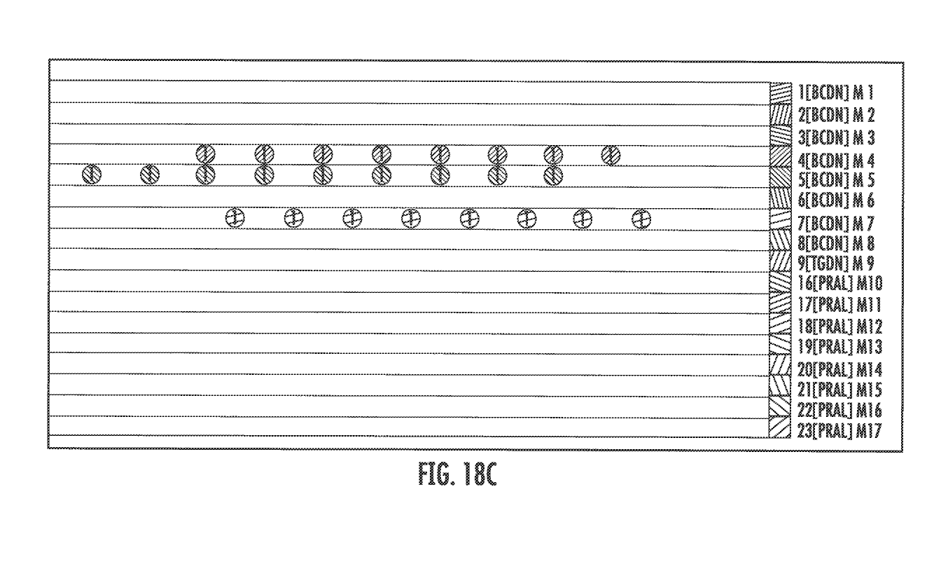

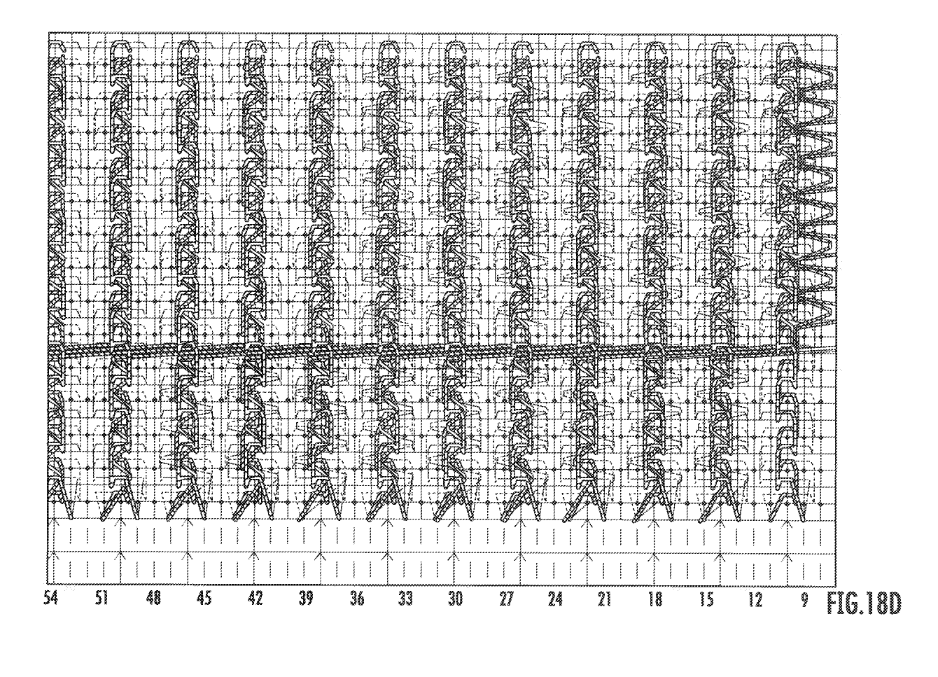

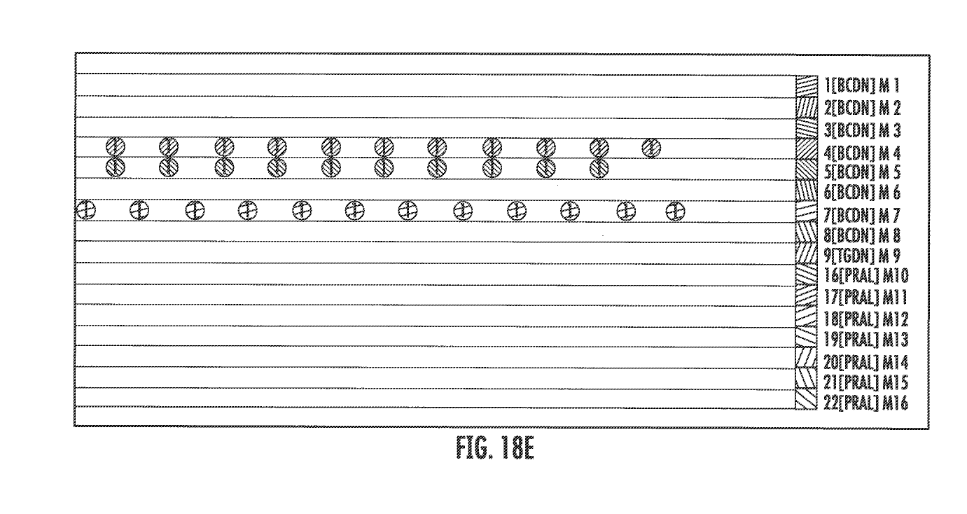

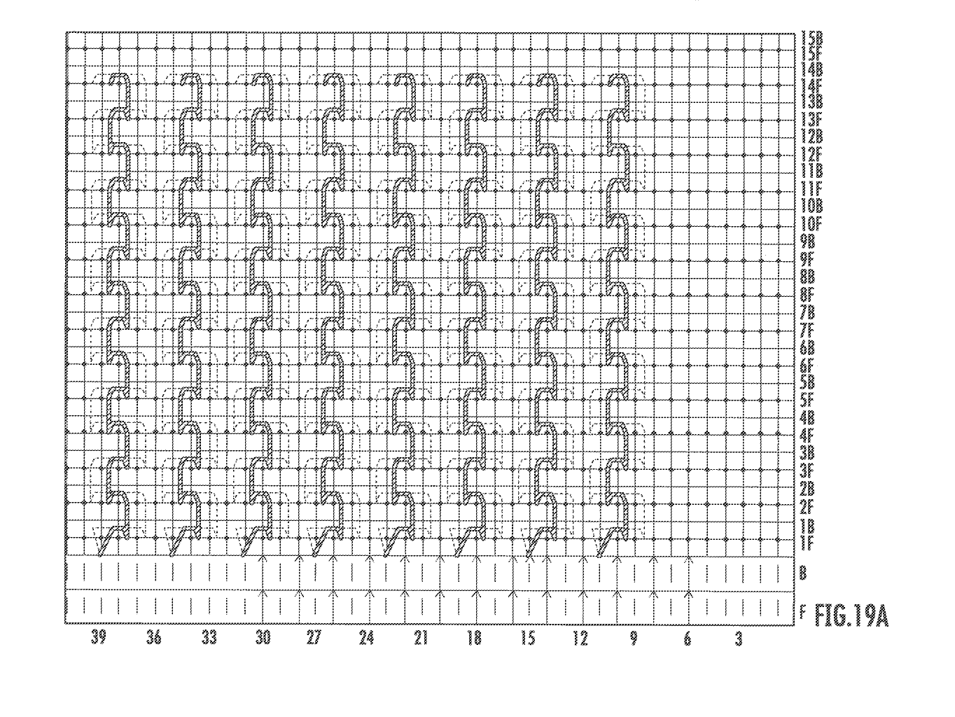

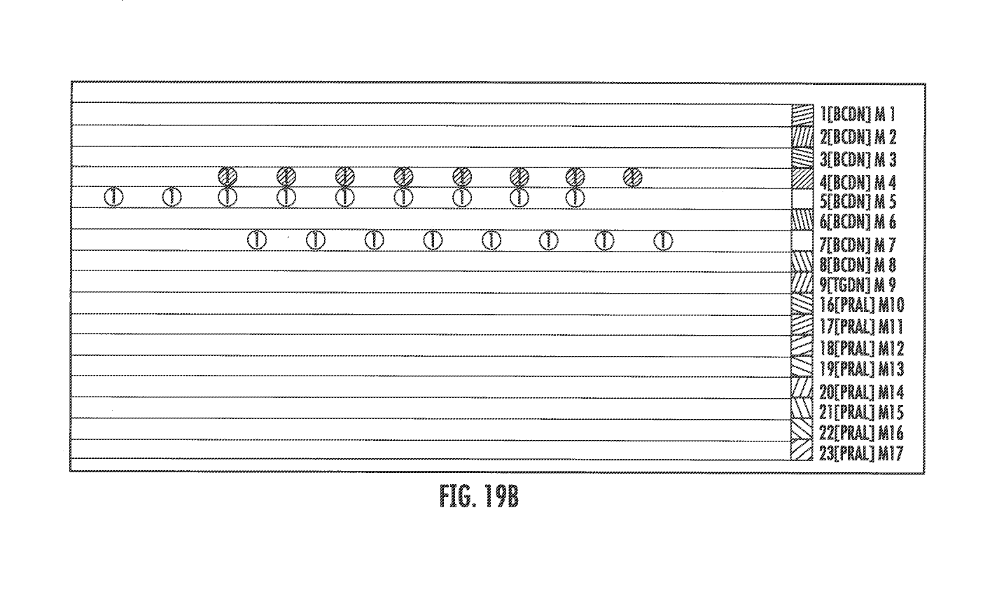

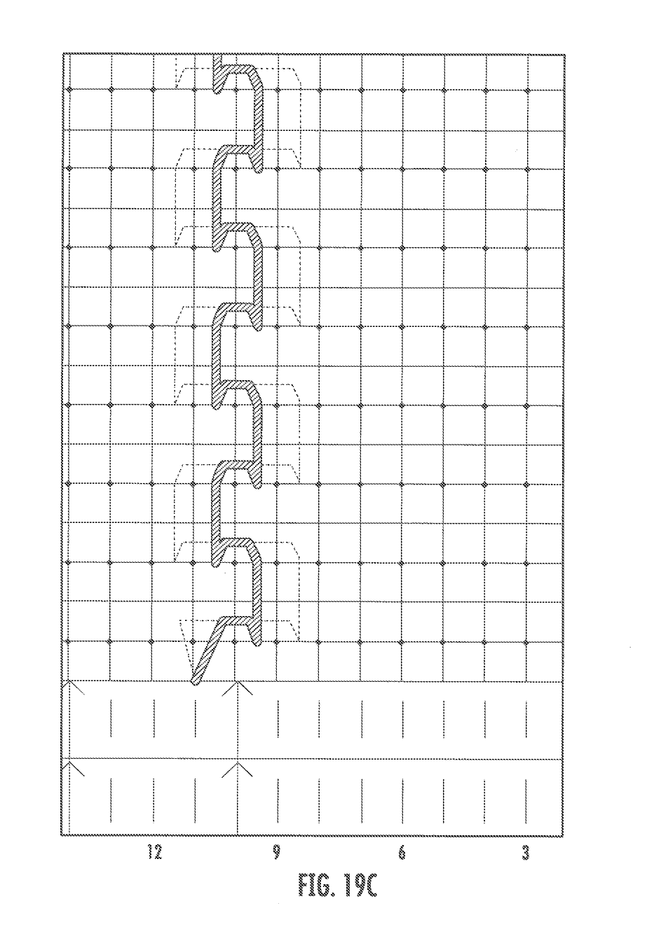

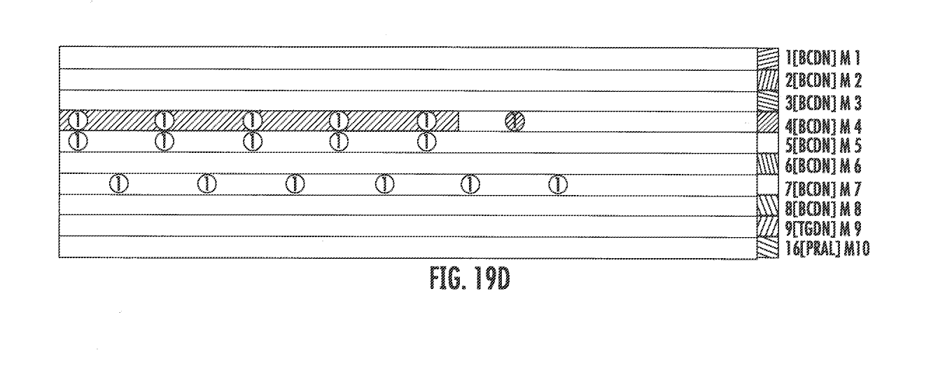

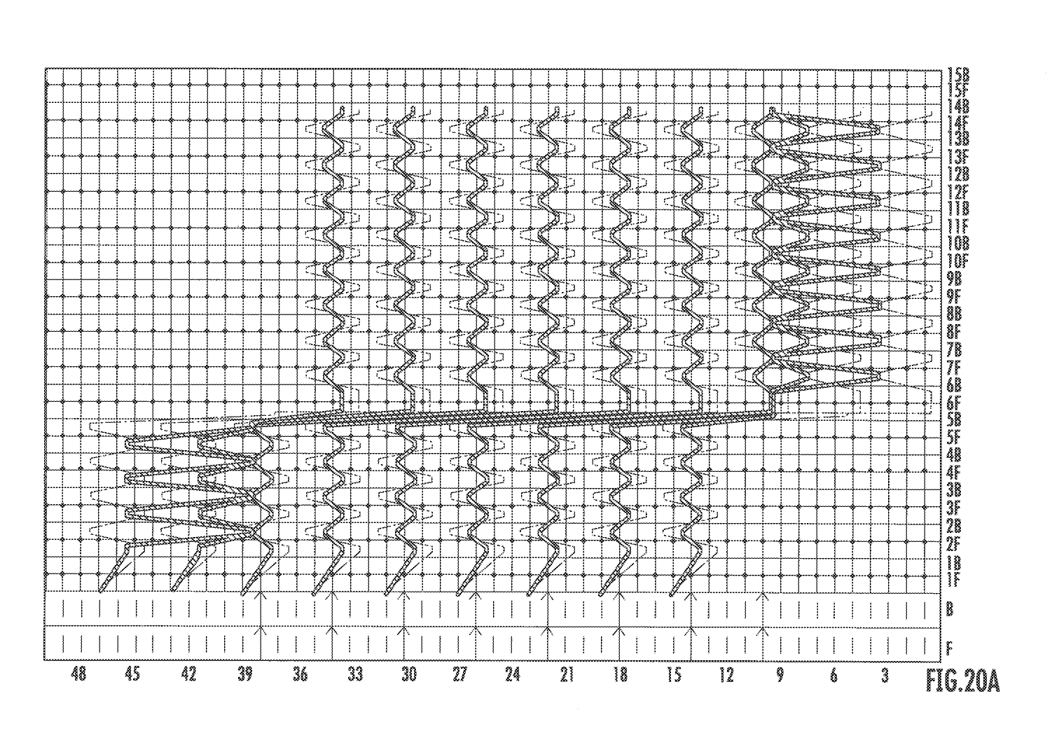

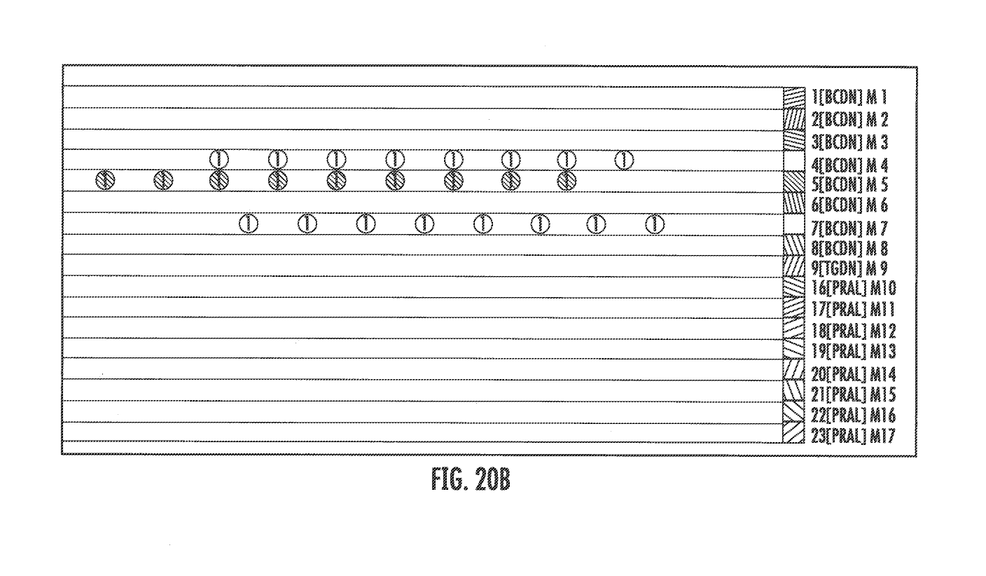

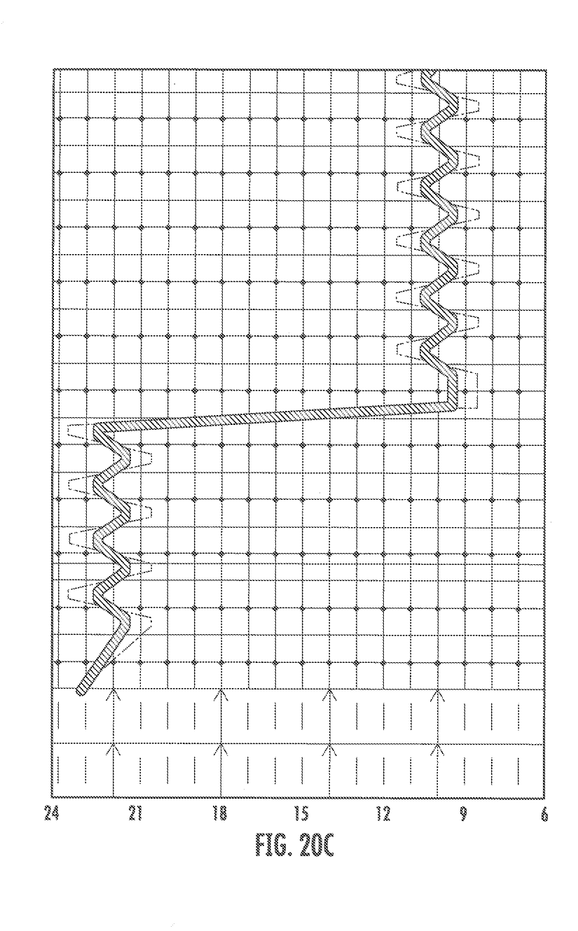

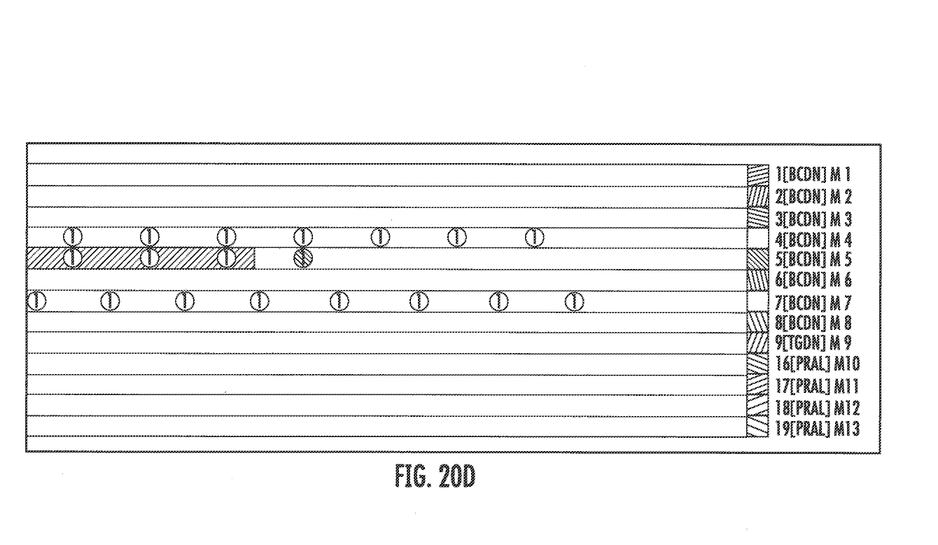

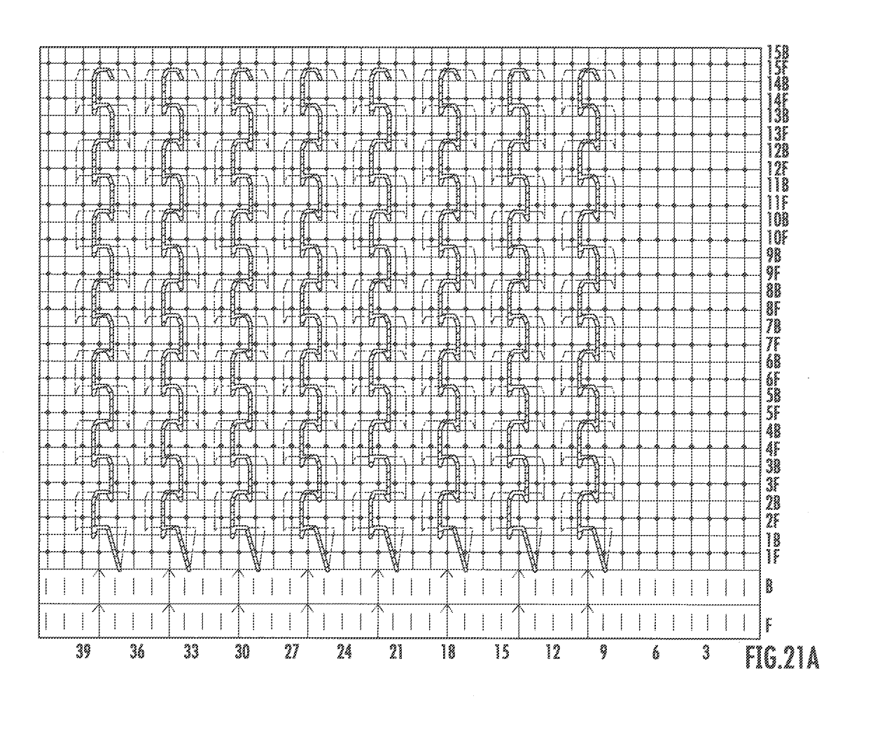

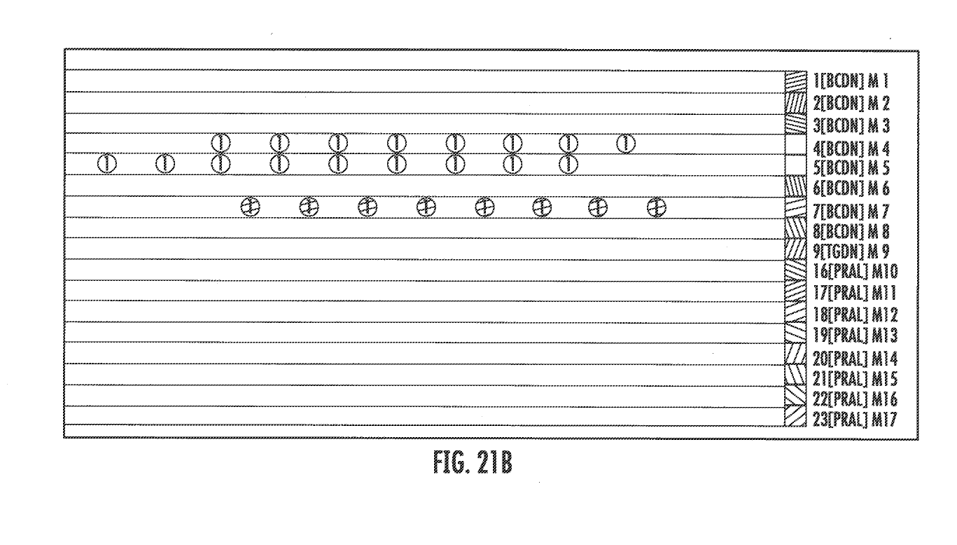

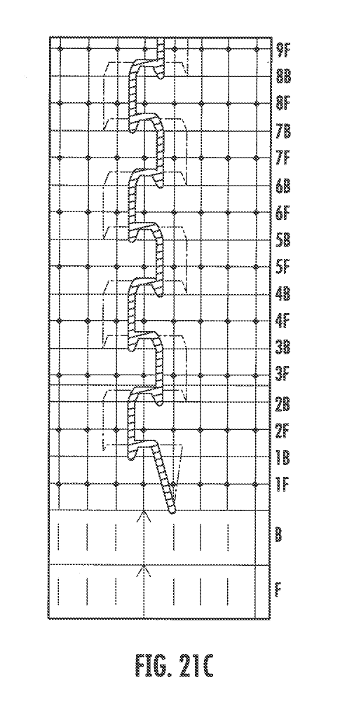

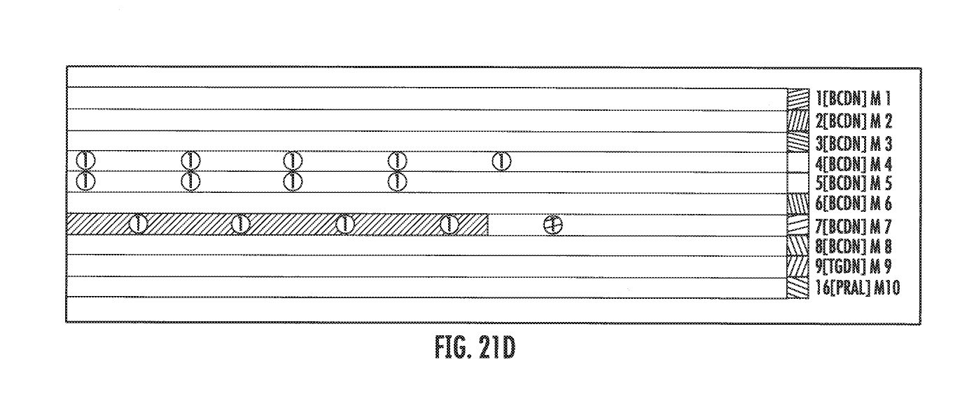

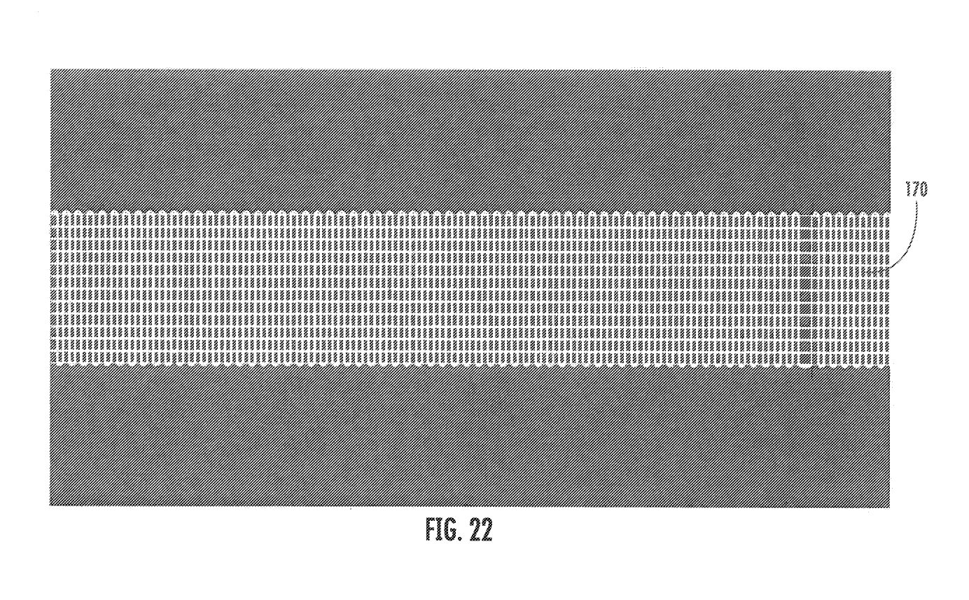

FIG. 18A is a photograph of a pattern layout for a silk-based scaffold design in accordance with the present invention. In FIG. 18A item 160 is a mesh or scaffold. This variation of the scaffold in accordance with another aspect of the present invention is preferably created on a raschel knitting machine such as Comez DNB/EL-800-8B set up in 5 gg needle spacing by the use of three movements as shown in the pattern layout in FIGS. 18B-E: two movements in the wale direction, the vertical direction within the fabric, and one movement in the course direction, the horizontal direction of the fabric. The movements in the wale direction occur on separate needle beds with alternate yarns; loops that occur on every course are staggered within repeat. The yarn follows a repeat pattern of 3/1-1/1-1/3-3/3-for one of the wale direction movements as shown in FIGS. 19A-D and 1/1-1/3-3/3-3/1 for the other wale direction movement as shown in FIG. 21A-D. The interlacing of the loops within the fabric allows for one yarn to be under more tension than the other under stress, locking it around the less tensioned yarn; keeping the fabric from unraveling when cut. The other movement in the course direction as shown in FIG. 20A-D occurs in every few courses creating the porous design. These yarns follow a repeat pattern of 15/15-15/15-13/13-15/15-13/13-15/15-13/13-15/15-13/13-15/15/-1/1-1/1-3/3-1/1-3/3-1/1-3/3-1/1-3/3-1/1 for the course direction movement. The pattern simulation layout of this pattern is rendered with ComezDraw 3 software in FIG. 22 considering a yarn design made with 2 ends of Td 20/22 raw silk twisted together in the S direction to form a ply with 6 tpi and further combining three of the resulting ply with 3 tpi for the two movements in the wale direction. For the movements in the course direction the yarn design is made with 3 ends of Td 20/22 raw silk twisted together in the S direction to form a ply with 6 tpi and further combining three of the resulting ply with 3 tpi. The stitch density or pick count for the design in FIG. 22 is 40 picks per centimeter considering the total pick count for the technical front face and the technical back face of the fabric, or 20 picks per cm considering only on the face of the fabric. The operating parameters are not limited to these described in FIGS. 18B-E, but just the optimum values for the specific yarn design used for the pattern simulation layout of FIG. 22. In FIG. 22 item 170 is a simulated double needle bed mesh or scaffold.

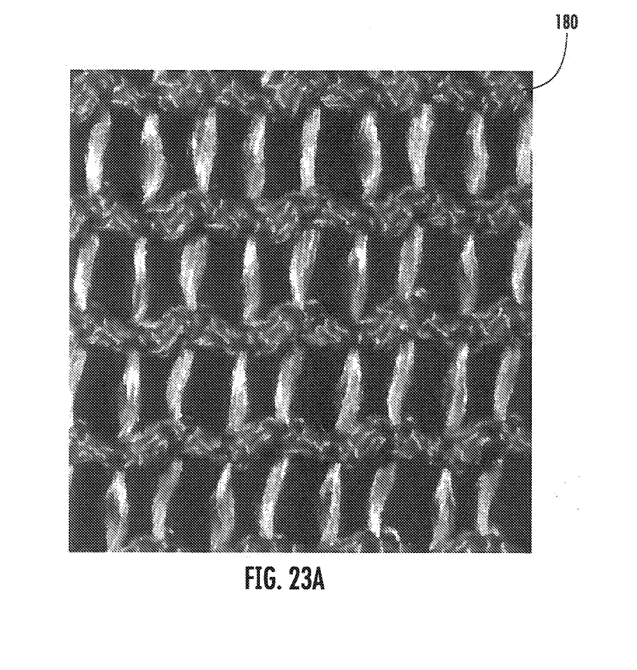

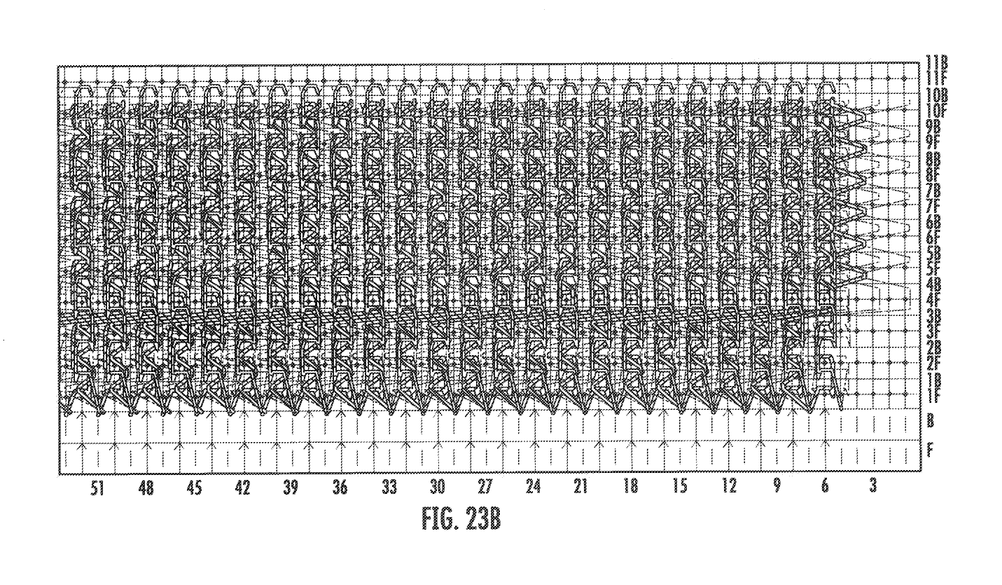

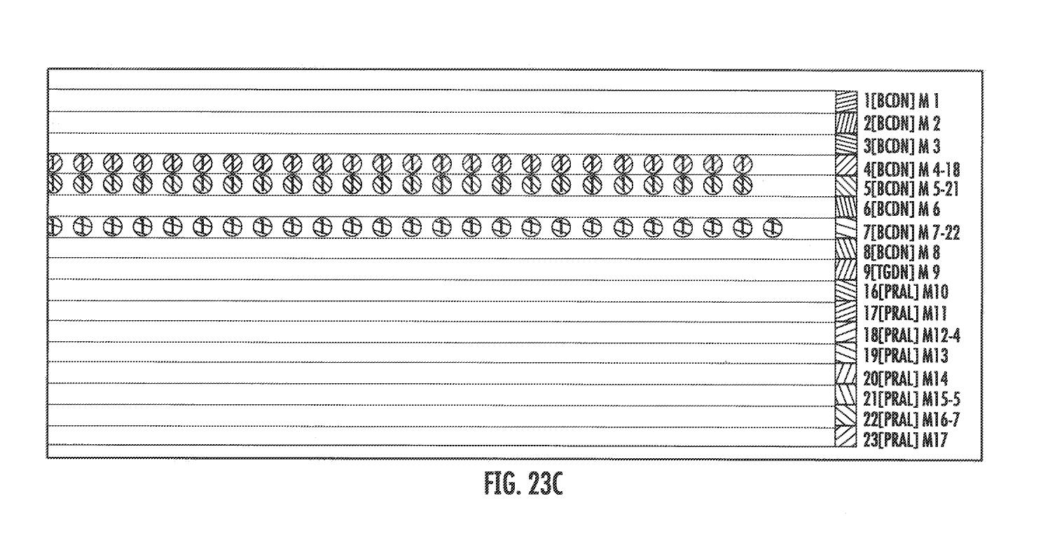

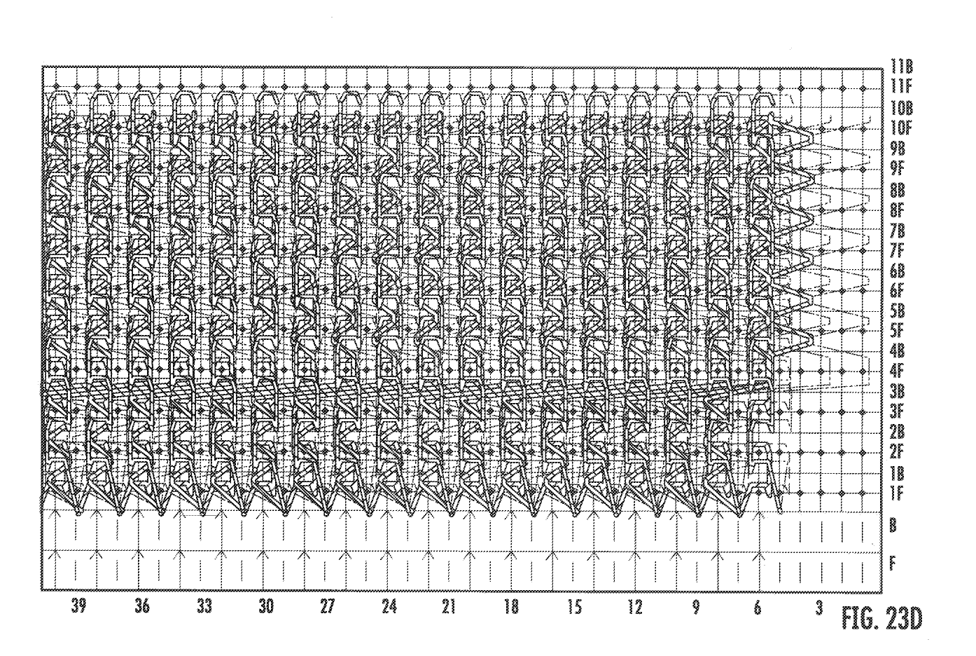

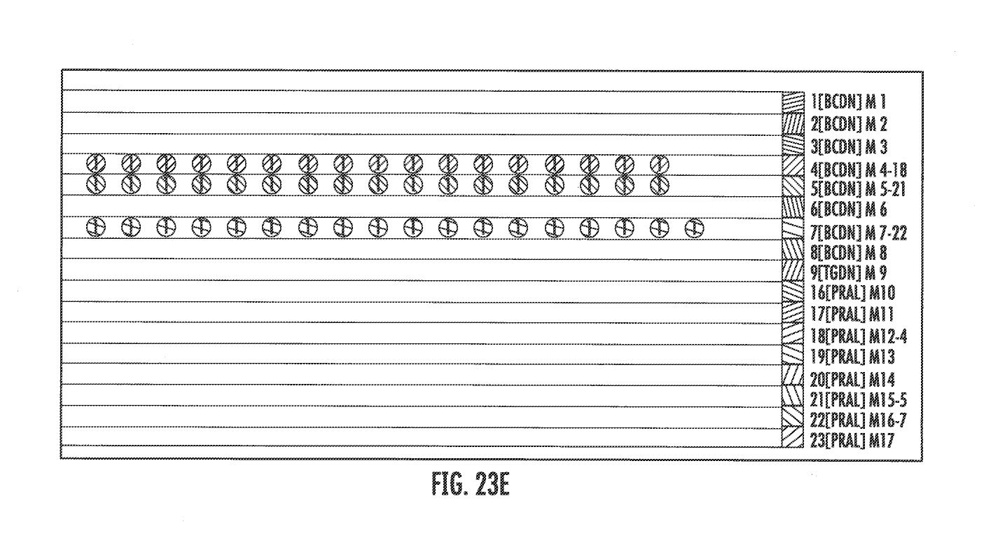

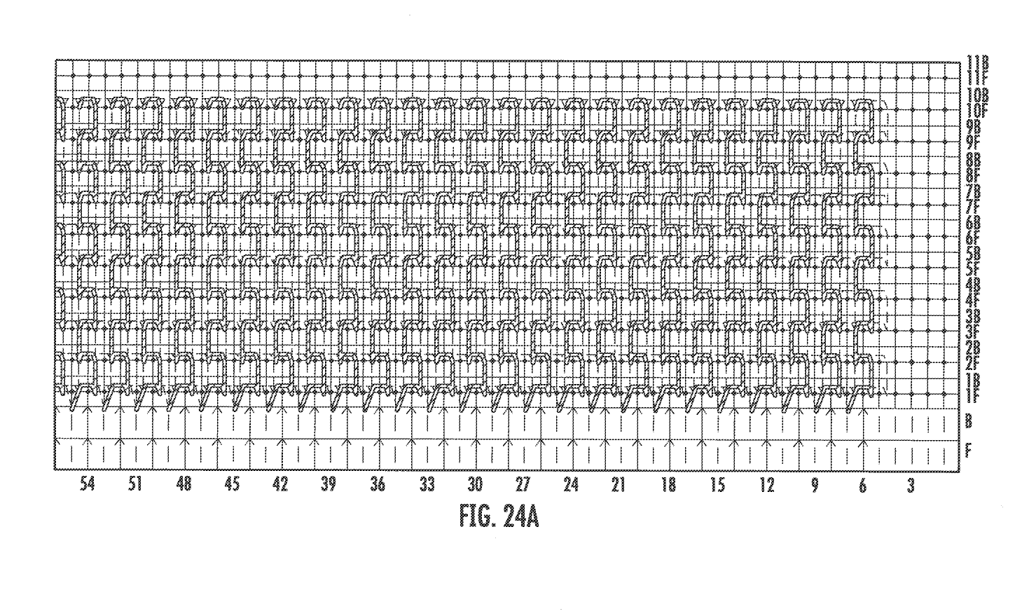

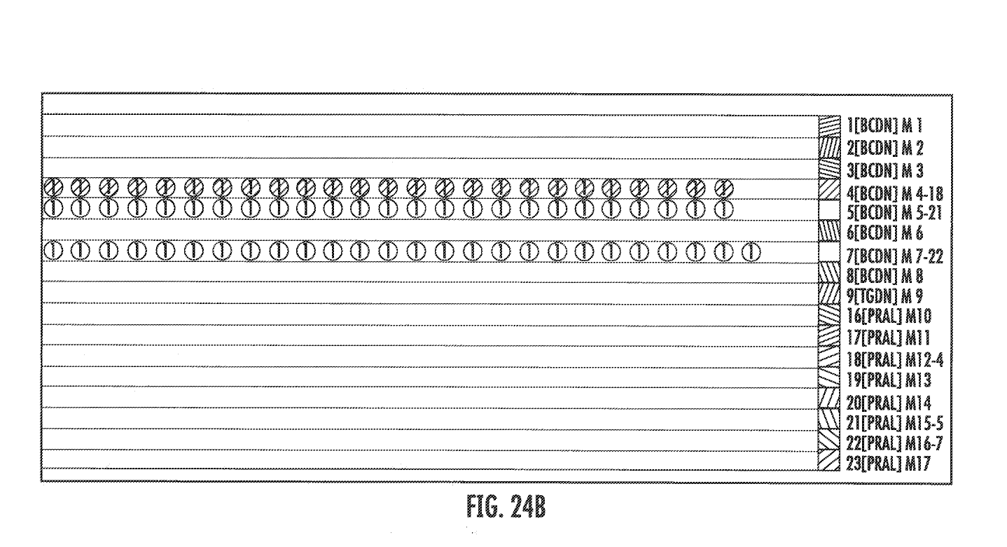

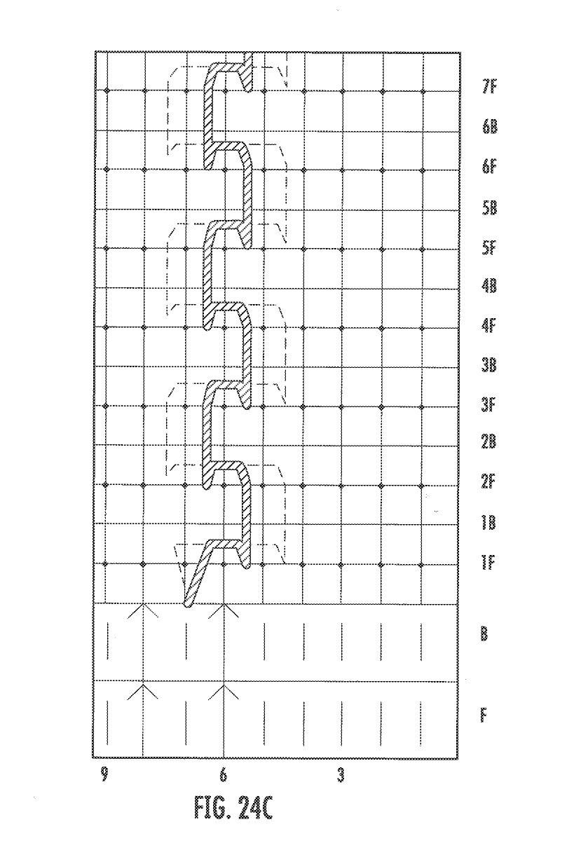

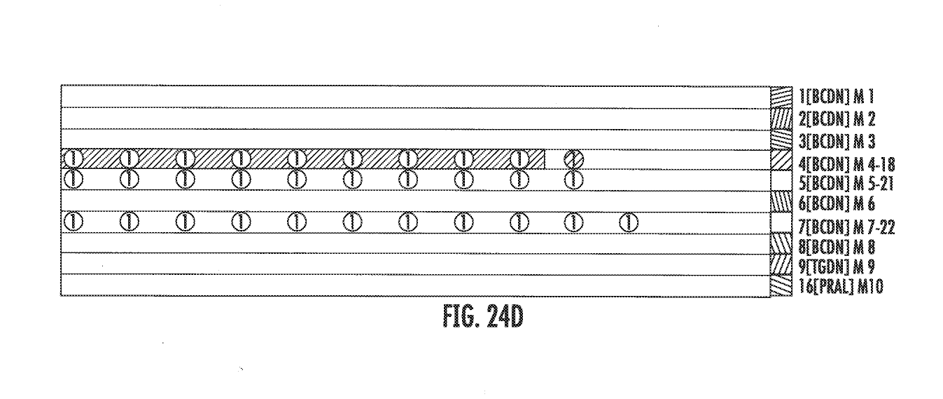

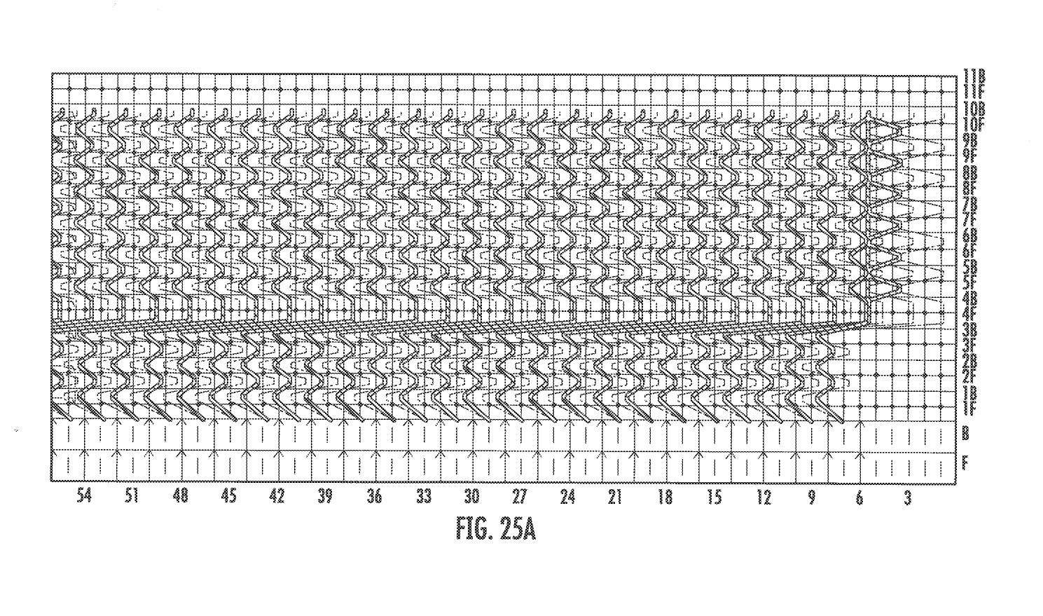

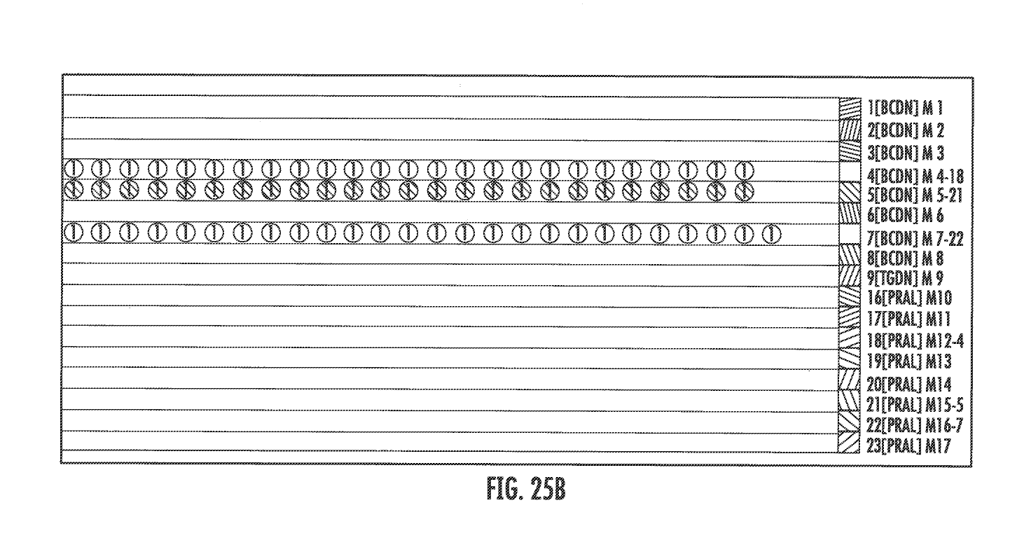

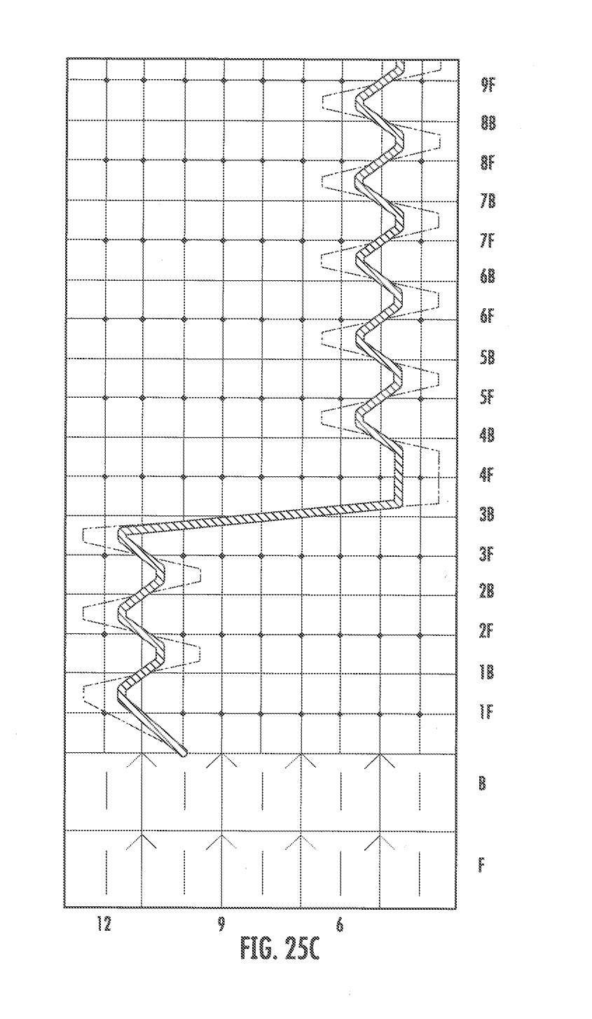

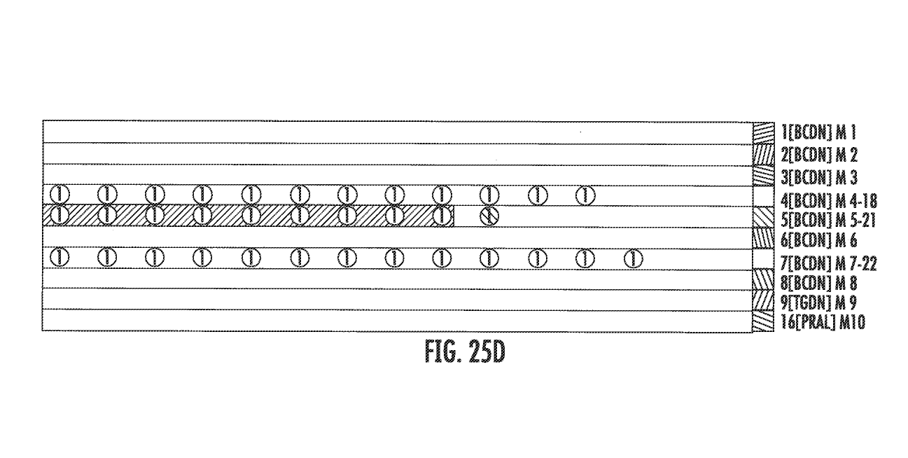

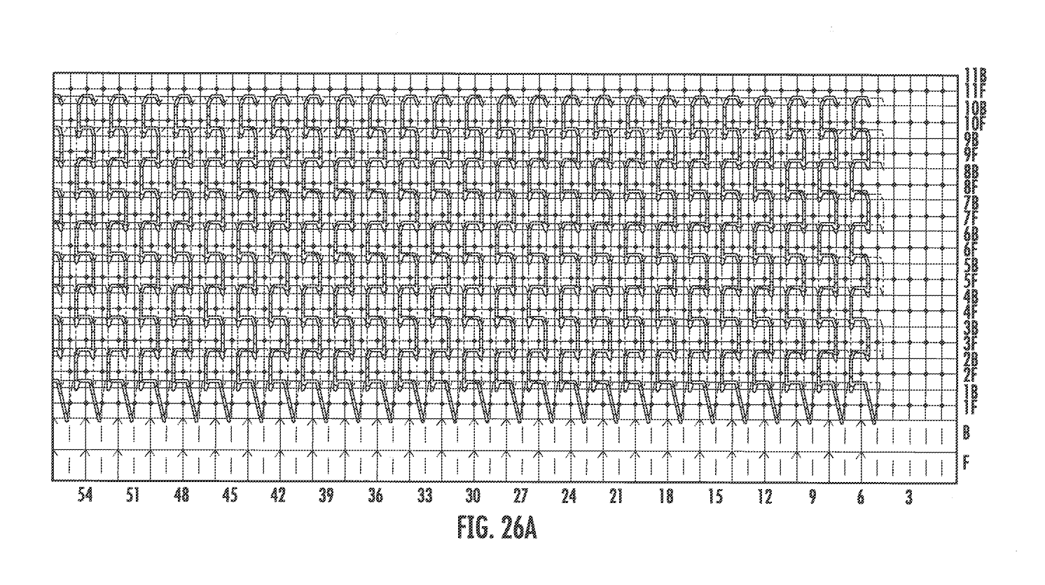

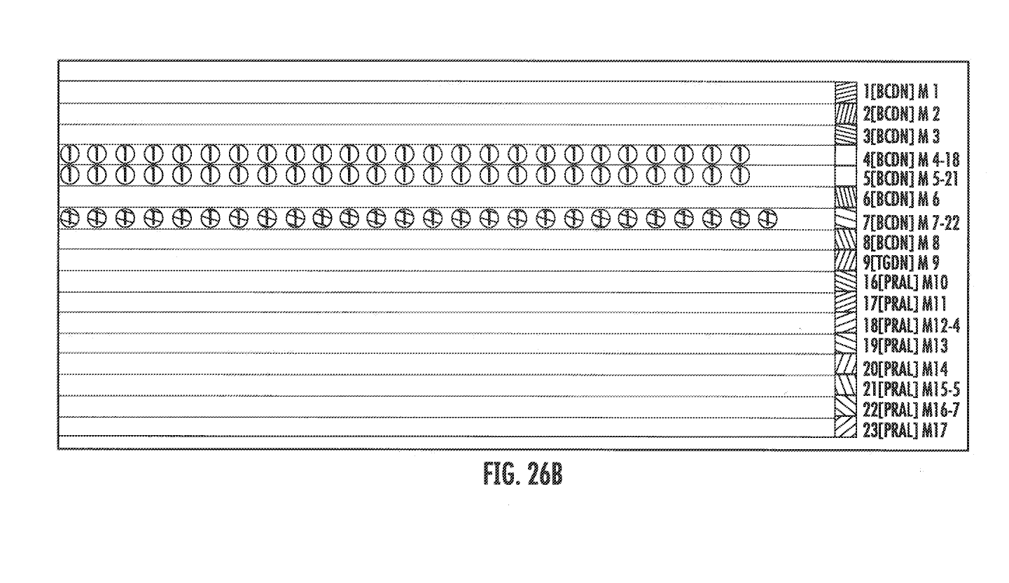

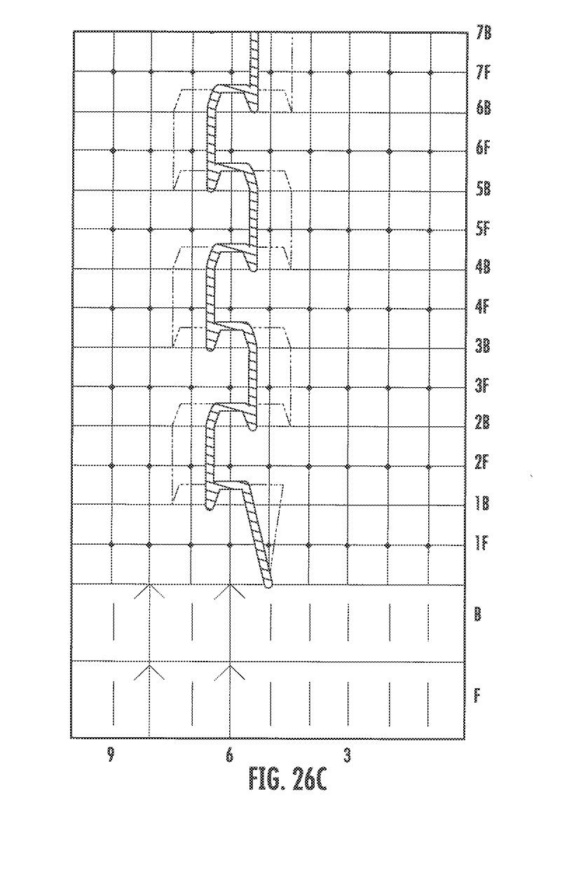

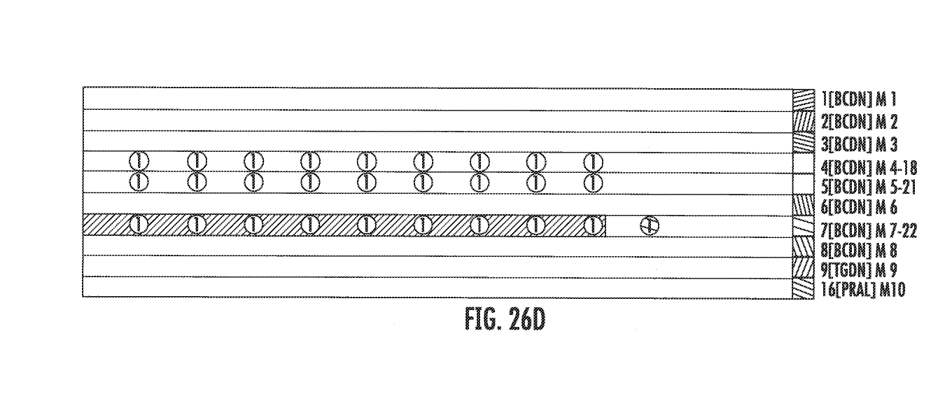

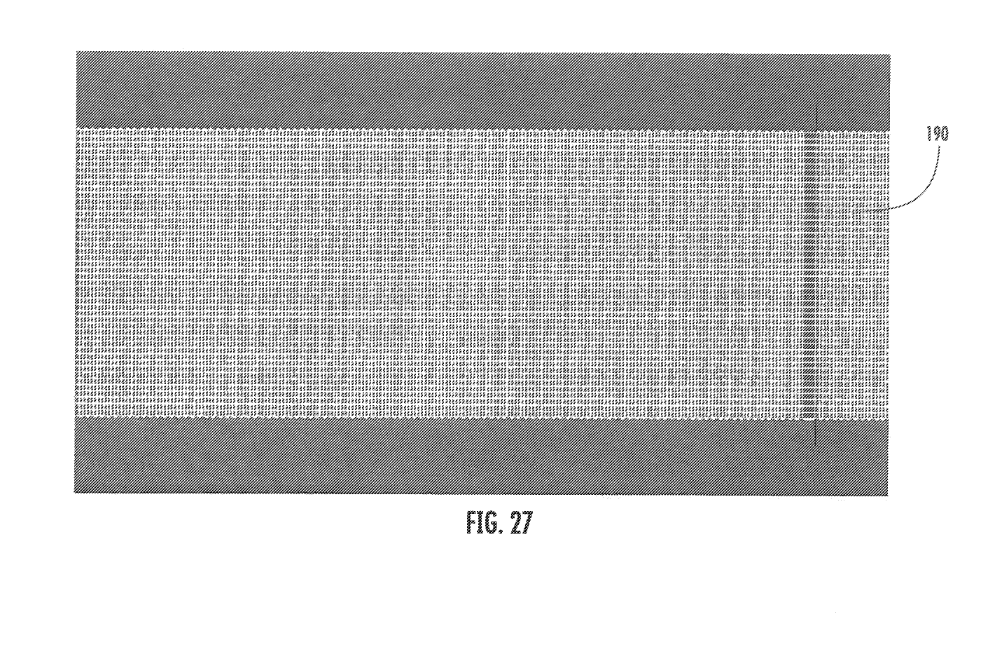

FIG. 23A is a photograph of a pattern layout for a silk-based scaffold design in accordance with the present invention. In FIG. 23A item 180 is a mesh or scaffold. This variation of the scaffold in accordance with an aspect of the present invention is preferably created on a raschel knitting machine such as Comez DNB/EL-800-8B set up in 10 gg needle spacing by the use of three movements as shown in the pattern layout in FIGS. 23B-E: two movements in the wale direction, the vertical direction within the fabric, and one movement in the course direction, the horizontal direction of the fabric. The movements in the wale direction occur on separate needle beds with alternate yarns; loops that occur on every course are staggered within repeat. The yarn follows a repeat pattern of 3/1-1/1-1/3-3/3-for one of the wale direction movements shown in FIGS. 24A-D and 1/1-1/3-3/3-3/1 for the other wale direction movement shown in FIGS. 26A-D. The interlacing of the loops within the fabric allows for one yarn to be under more tension than the other under stress, locking it around the less tensioned yarn; keeping the fabric from unraveling when cut. The other movement in the course direction as shown in FIGS. 25A-D occurs in every few courses creating the porous design. These yarns follow a repeat pattern of 9/9-9/9-7/7-9/9-7/7-9/9-7/7-9/9-7/7-9/9/-1/1-1/1-3/3-1/1-3/3-1/1-3/3-1/1-3/3-1/1 for the course direction movement. The pattern simulation layout of this pattern is rendered with ComezDraw 3 software in FIG. 27 considering a yarn design made with 2 ends of Td 20/22 raw silk twisted together in the S direction to form a ply with 6 tpi and further combining three of the resulting ply with 3 tpi. The same yarn design is used for the movements occurring in the wale and course directions. The stitch density or pick count for the design in FIG. 27 is 40 picks per centimeter considering the total picks count for the technical front and the technical back of the fabric, or 20 picks per cm considering only on the face of the fabric. The operating parameters are not limited to the those described in FIGS. 23B-E, but just the optimum values for the specific yarn design used for the pattern simulation layout of FIG. 27. In FIG. 27 item 190 is a simulated double needle bed mesh or scaffold.

In embodiments employing silk yarn, the silk yarn may be twisted from yarn made by 20-22 denier raw silk fibers approximately 40 to 60 μm in diameter. Preferably, raw silk fibers ranging from 10 to 30 deniers may be employed; however any fiber diameters that will allow the device to provide sufficient strength are acceptable. Advantageously, a constant yarn size may maximize the uniformity of the surgical scaffold mechanical properties, e.g. stiffness, elongation, etc., physical and/or biological properties within each region. However, the yarn size may be varied in sections of the scaffold in order to achieve different mechanical, physical and/or biological characteristics in the preferred scaffold locations. Factors that may be influenced by the size of the yarn include, but are not limited to: ultimate tensile strength (UTS); yield strength, i.e. the point at which yarn is permanently deformed; percent elongation; fatigue and dynamic laxity (creep); bioresorption rate; and transfer of cell/nutrients into and out of the mesh.

The knit patterns illustrated in FIGS. 1A, 2A, 7A, 13A, 18A and 23A respectively, may be knit to any width depending upon the knitting machine and could be knitted with any of the gauges available with the various crochet machines or warp knitting machines. Table 1 outlines the fabric widths that may be achieved using a different numbers of needles on different gauge machines. It is understood that the dimensions in Table 1 are approximate due to the shrink factor of the knitted fabric which depends on stitch design, stitch density, and yarn size used.

TABLE 1

| | |
|---|---|
| Needle Count | Knitting Width (mm) |

| Gauge | From | To | From | To |
|---|---|---|---|---|
| 48 | 2 | 5656 | 0.53 | 2997.68 |
| 24 | 2 | 2826 | 1.06 | 2995.56 |
| 20 | 2 | 2358 | 1.27 | 2994.66 |
| 18 | 2 | 2123 | 1.41 | 2993.43 |
| 16 | 2 | 1882 | 1.59 | 2992.38 |
| 14 | 2 | 1653 | 1.81 | 2991.93 |
| 12 | 2 | 1411 | 2.12 | 2991.32 |
| 10 | 2 | 1177 | 2.54 | 2989.58 |
| 5 | 2 | 586 | 5.08 | 2976.88 |

Embodiments of the scaffold device according to the present invention may be knitted on a fine gauge crochet knitting machine. A non-limiting list of crochet machines capable of manufacturing the surgical scaffold according to aspects of the present invention are provided by: Changde Textile Machinery Co., Ltd.; Comez; China Textile Machinery Co., Ltd.; Huibang Machine; Jakob Muller AG; Jingwei Textile Machinery Co., Ltd.; Zhejiang Jingyi Textile Machinery Co., Ltd.; Dongguan Kyang Yhe Delicate Machine Co., Ltd.; Karl Mayer; Sanfang Machine; Sino Techfull; Suzhou Huilong Textile Machinary Co., Ltd.; Taiwan Giu Chun Ind. Co., Ltd.; Zhangjiagang Victor Textile; Liba; Lucas; Muller Frick; and Texma.

Embodiments of the scaffold device according to the present invention may be knitted on a fine gauge warp knitting machine. A non-limiting list of warp knitting machines capable of manufacturing the surgical mesh according to aspects of the present invention are provided by: Comez; Diba; Jingwei Textile Machinery; Liba; Lucas; Karl Mayer; Muller Frick; Runyuan Warp Knitting; Taiwan Giu Chun Ind.; Fujian Xingang Textile Machinery; and Yuejian Group.

Embodiments of the scaffold device according to the present invention may be knitted on a fine gauge flat bed knitting machine. A non-limiting list of flat bed machines capable of manufacturing the surgical mesh according to aspects of the present invention are provided by: Around Star; Boosan; Cixing Textile Machine; Fengshen; Flying Tiger Machinery; Fujian Hongqi; G & P; Gorteks; Jinlong; JP; Jy Leh; Kauo Heng Co., Ltd.; Matsuya; Nan Sing Machinery Limited; Nantong Sansi Instrument; Shima Seiki; Nantong Tianyuan; and Ningbo Yuren Knitting.

Method Of In Vivo Evaluation Of Medical Device Scaffolds

Implantation of sub-mammary tissue expanders, augmentation devices, and reconstruction materials is a common feature of both non-pathologic breast augmentation and post-mastectomy breast reconstruction in humans. In either case, translocated muscle or skin flaps or non-patient biological materials of a sufficient size are implanted to accommodate placement of the tissue expanders or augmentation devices. To date, no animal studies that implement anatomical locations sufficiently similar to breast reconstruction have been published to assess functionality and biological response to implanted tissue expanders or supplemental materials appropriate for breast reconstruction. In an aspect of the present invention, a quadruped is used for a model system. The term “model” or “modeling”, as used herein, means mimicking or simulating what would occur in a human using a quadraped. For example, the method of the present invention comprises modeling of soft tissue repair such as, for example, in human breast reconstruction. Thus, method for modeling soft tissue repair in a human in accordance with the present invention comprises implanting a medical device scaffold in a quadruped. The present invention is directed to a suitable animal model for this human end use applications and the surgical procedure.

Quadrupeds including, but not limited to, sheep and pigs are among the animal species suitable for use in the animal model of the present invention. The animal model comprises sub-lattisimus dorsi muscle (SLDM) implantation of breast reconstruction/augmentations devices. Although the SLDM muscle is suitable, any other muscle that allows the positioning of the implant may be used in accordance with the present invention. The lattisimus dorsi muscle in mature (i.e. not fully grown) sheep and pigs has a shape, orientation, and size similar to the human pectoralis major muscle. In accordance with the method of the present invention, the species, breed, animal size, tissue expander (TE) size, and surgical technique were selected.

The implantable devices of the present invention and the in vivo animal model of the present invention for simulating human implantation of such devices is suitable for use in a variety or reconstructive or support applications including, but not limited to, breast reconstruction, mastoplexy, breast augmentation revision, breast augmentation support, standard breast augmentation, chest wall repair, organ support, body contouring, abdominoplasty, facial reconstruction, hernia repair, and pelvic floor repair.

### EXAMPLES

The following Examples illustrate aspects of the present invention

### Example 1

Sheep Study to Determine Suitability of the Silk Scaffold in Breast Reconstruction

A study was conducted to evaluate the performance and suitability of surgical scaffold devices within the scope of the present invention by implanting them in simulated human breast reconstruction procedures using an in vivo sub-latissimus dorsi muscle implantation model in sheep. Specifically, the study evaluated the biological response to, and explant characteristics of, a variety of scaffold configurations when employed in a clinically relevant manner and determined that the silk scaffold is well suited for use in human breast reconstruction surgery and procedures. The test system was as follows:

Test System: Animals (Sheep)

The test animals were sheep (Ovis aries) of the strain or Breed rambouillet cross or Suffolk-Hampshire cross. There were 96 test animals and 10-auxiliary animals. The animals (i.e. the sheep) were castrated males or not pregnant females. The age at of the animals at the time of surgery was 9 to 16 months. The weight of the animals at the time of the surgery: was 36 kg to 60 kg.

### Test Articles

The test articles (the “Test groups” below) used in this study were the silk surgical mesh (scaffold), also referred to as devices or “device”, also referred to as SeriScaffold™, all being within the scope of the present invention. The ratio of the implantable test article weight (surgical mesh) to the weight of the animal ranged between 7 mg to 46 mg per kg of animal body weight for 36-60 kg sheep, respectively.

The test article used was a surgical mesh was indicated (FDA approved) for use as a transitory scaffold for soft tissue support and repair to reinforce deficiencies where weakness or voids existed that required the addition of material to obtain the desired surgical outcome. Additionally, all the test articles used were knitted (or synonymously knit), multi-filament, bioengineered, silk mesh which has the properties of being mechanically strong, biocompatible, and long term bioresorbable.

Test group—Silk-Based Scaffold Design No. 3 (Shown in FIG. 7A)

Test group—Silk-Based Scaffold Design No. 5 (Shown in FIG. 18A)

Test group—Silk-Based Scaffold Design No. 6 (Shown in FIG. 23A)

Test group—Silk-Based Scaffold Design No. 1 (See FIG. 1A).

Sterile technique was used when handling all test articles prior to and during implantation.

The device implants (that is the test articles used) were extensively irrigated and aspirated with saline/antibiotic solution following the in situ cutting of the device to remove any device particulate debris that was generated.

### Tissue Expander and Breast Implants

This study included use of NATRELLE™ 133 Anatomical Tissue Expanders and either NATRELLE™ silicone-filled smooth round breast implants or NATRELLE™ silicone-filled BIOCELL® textured round breast implants.

NATRELLE™ 133 Anatomical Tissue Expanders

Size: 500-750 cc

Manufacturer: Allergan Medical

The 133 Series tissue expander used was intended for temporary subcutaneous implantation and required periodic, incremental inflation with sterile saline for injection until the desired degree of tissue expansion was achieved.

The 133 Series tissue expanders were constructed from silicone elastomer and consisted of an expansion envelope with a BIOCELL® textured surface, and a MAGNA-SITE® integrated injection site. The expanders were available in a wide range of styles and sizes. The indications for use were as follows: breast reconstruction following mastectomy; treatment of underdeveloped breasts; treatment of soft tissue deformities.

NATRELLE™ silicone filled smooth or textured round breast Implants 500-750 cc volume) available from Allergan Medical, Santa Barbara, Calif.

NATRELLE™ silicone-filled breast implants are made with barrier shell technology resulting in a low diffusion silicone elastomer shell and were filled with a soft, cohesive silicone gel. All styles used in this study were single “lumen” round design and consisted of a shell, a patch, and silicone gel filling. NATRELLE™ silicone-filled breast implants were dry heat sterilized. NATRELLE™ is approved (indicated) for breast augmentation for women at least 22 years old and for use in breast reconstruction.

### Study Design

Animals

Groups A, B, C, and D contained three (3) animals or 6 surgical sites for each of the following time points: 1, 3, 6, 12, 18, and 24 months. The study utilized up to 72 sheep (12 per time point) for groups A, B, C, D. Study groups (quantities are listed as per time point)

Test group—Silk-Based Scaffold Device No. 3 (3 sheep, bilateral procedure)

Test group—Silk-Based Scaffold Device No. 6 (3 sheep, bilateral procedure)

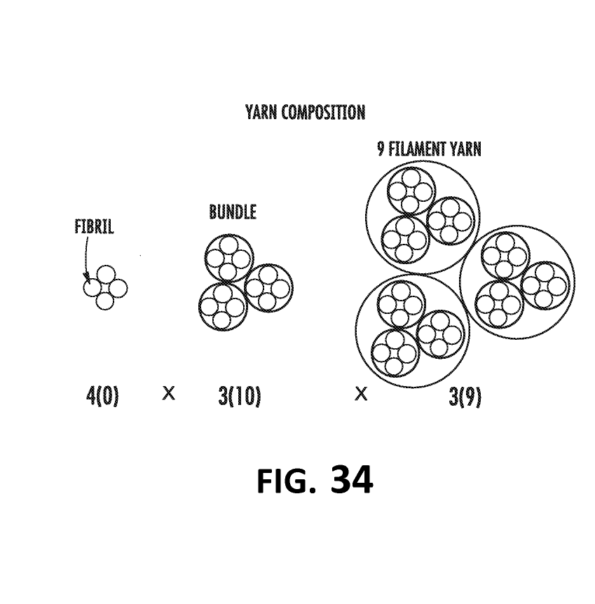

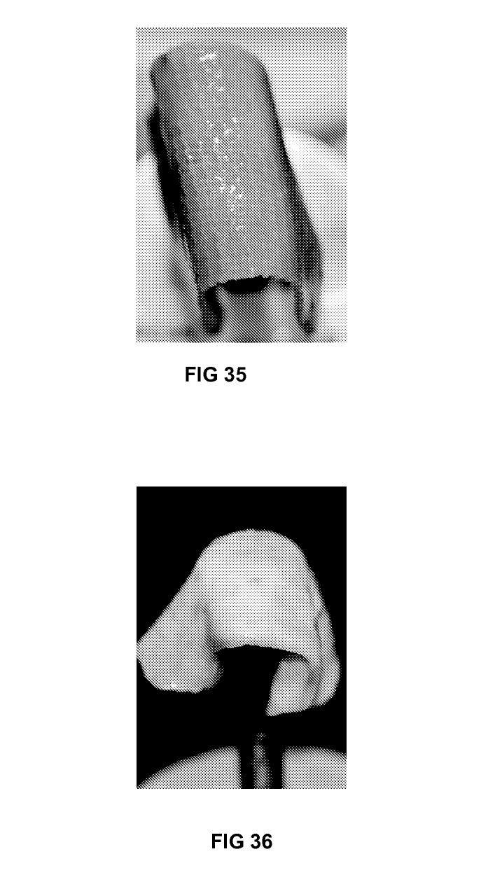

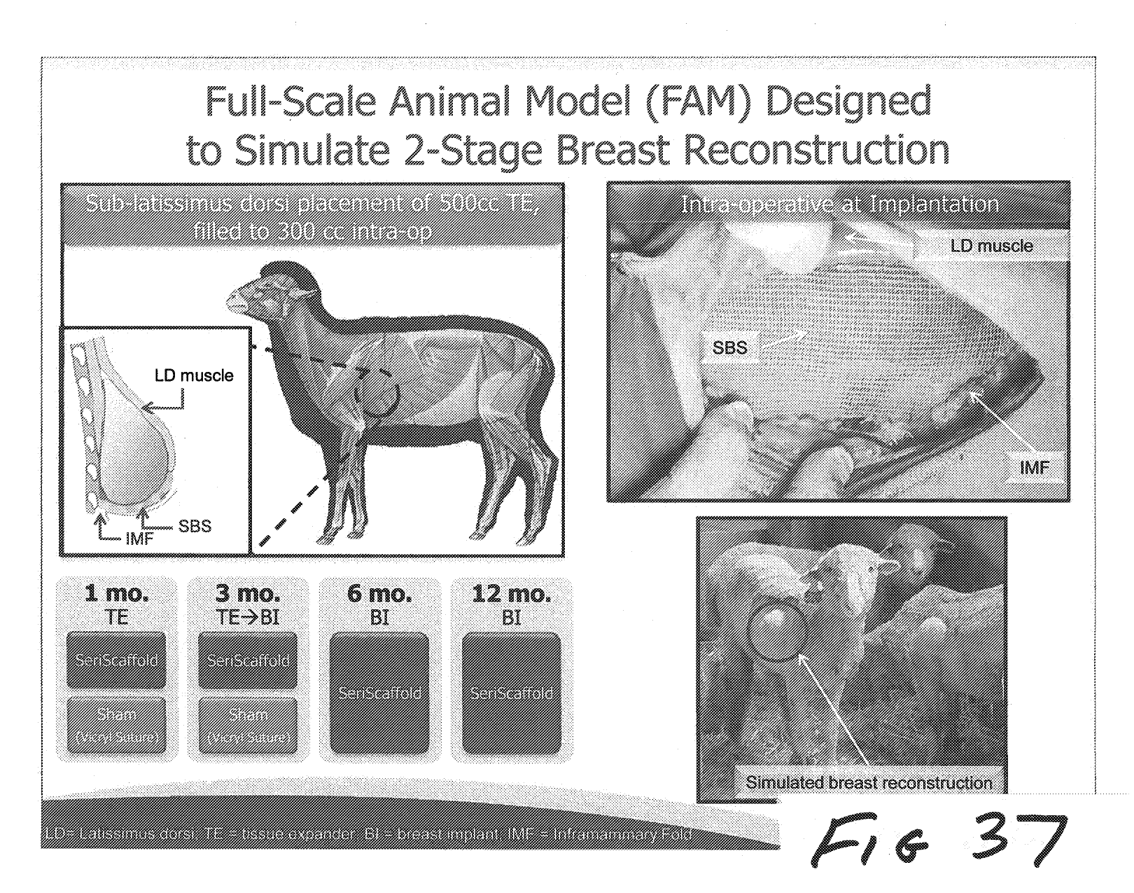

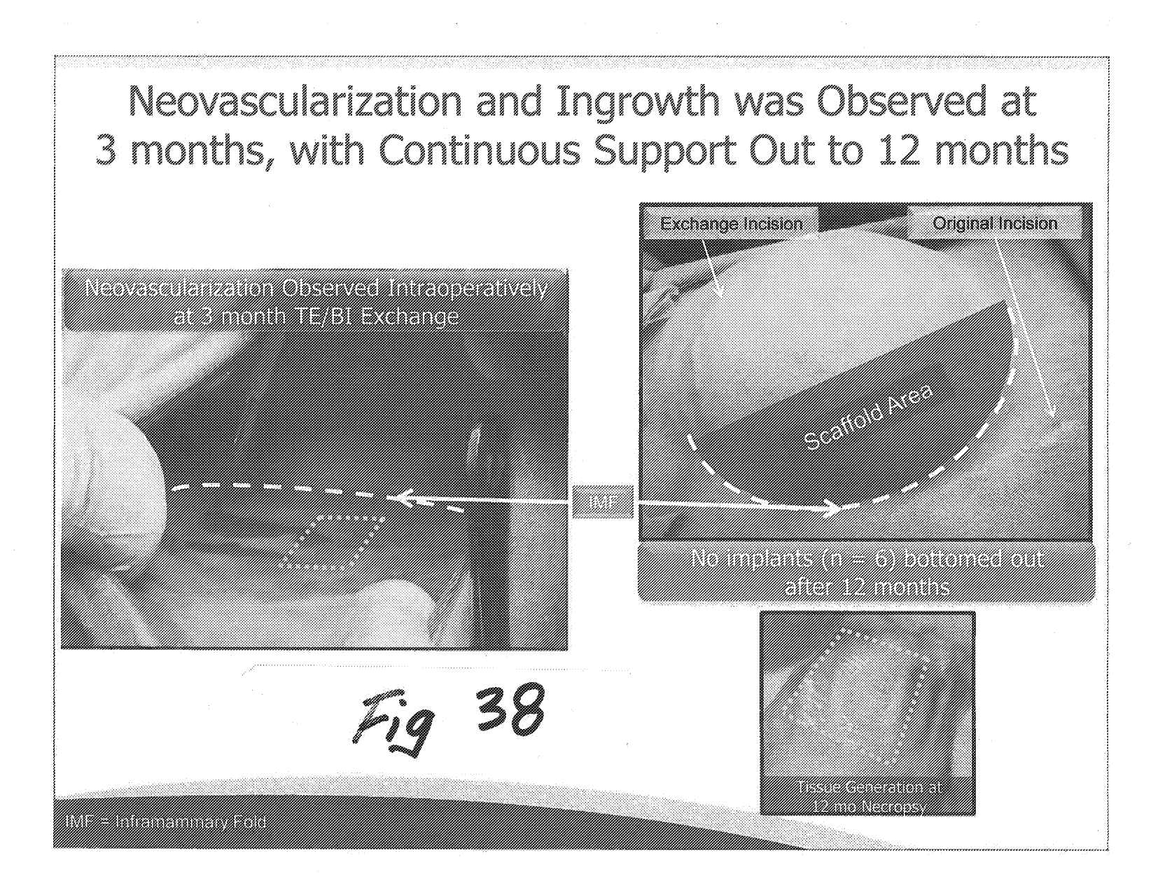

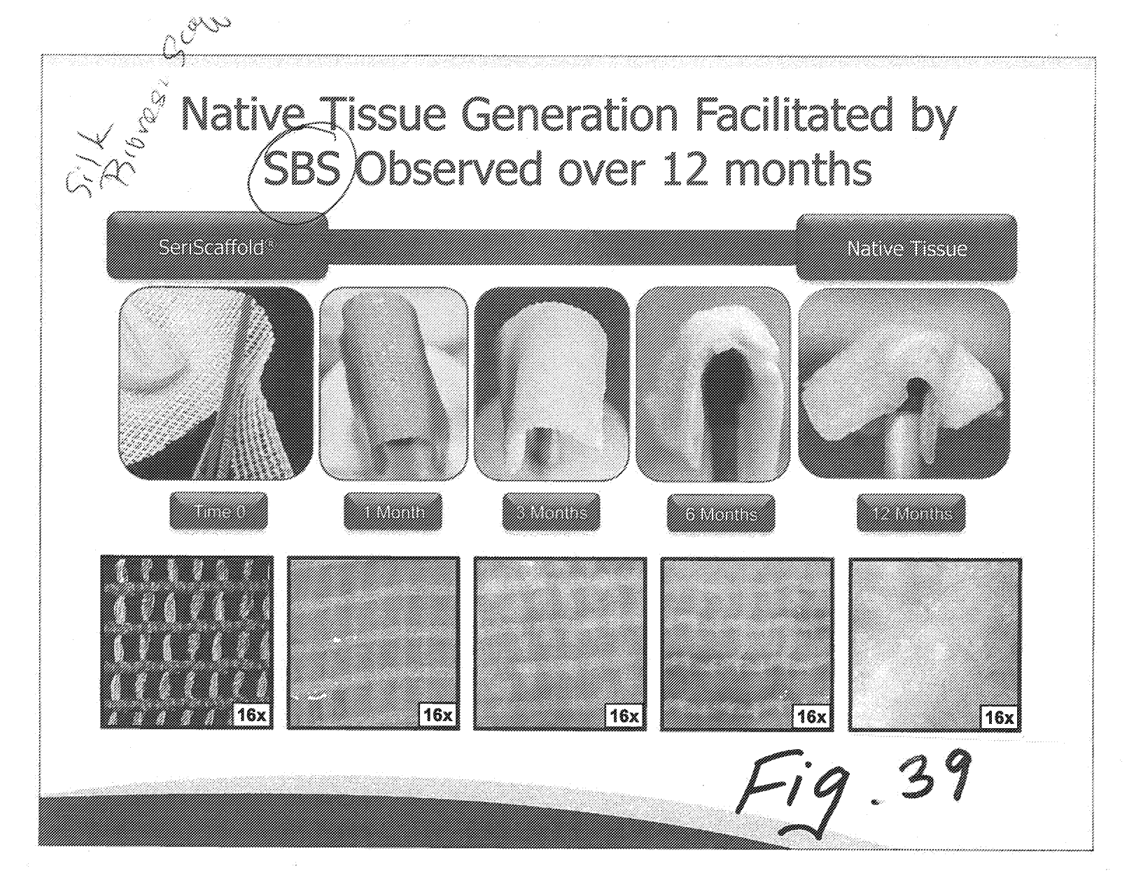

Test group—Silk-Based Scaffold Device No. 1 (3 sheep, bilateral procedure). Silk-Based Scaffold (SBM) No. 1 scaffold used was knit with 9-filament, twisted silk yarns. A yarn was comprised of three silk bundles, each of which was comprised of individual silk fibrils as illustrated in FIG. 34. The 9-filament yarns were knit into the surgical scaffold. The wales ran horizontally and the courses ran vertically along the scaffold.

Sham control group (3 sheep, bilateral procedure)

Study Metrics

Throughout the study, animals and surgical sites were examined by the following metrics:

Clinical observations (pre- and post-operative, and pre-necropsy)

Histological evaluation (post-necropsy)

Histomorphometry (post-necropsy)

Post-device explanation physical and biomechanical evaluation

Diagnostic imaging of the surgical site and surrounding tissue (in-life—at specified intervals)

Clinical pathology (in-life—at specified intervals)

### Study Schedule: Necropsy and Implant Exchange

One Month Sacrifice: 30±3 days post-operative

All animals from the 1 month group were euthanized and necropsied 30±3 days post-operative.

Three Month Sacrifice: 13±1 week post-operative

All animals from the 3 month group were euthanized and necropsied 13±1 week post-operative.

Tissue Expander to Implant Exchange: 13±2 weeks post-tissue expander implantation

All animals from the 6 and 12 month groups were surgically operated on to exchange the tissue expander for the breast implant 13±2 weeks post-tissue expander implantation.

Six Month Sacrifice: 26±2 weeks post-tissue expander implantation

All animals from the 6 month group were euthanized and necropsied 26±2 weeks post-tissue expander implantation.

Twelve Month Sacrifice: 52±2 weeks post-tissue expander implantation

All animals from the 12 month group were euthanized and necropsied 52±2 weeks post-tissue expander implantation.

Eighteen Month Sacrifice: 78±2 weeks post-tissue expander implantation

All animals from the 18 month group were euthanized and necropsied 78±2 weeks post-tissue expander implantation.

Twenty-Four Month Sacrifice: 104±2 weeks post-tissue expander implantation

All animals from the 24 month group were euthanized and necropsied 104±2 weeks post-tissue expander implantation.

### In-Vivo Procedures

Test articles, tissue expanders and breast implants were briefly immersed in a triple antibiotic solution consisting of 1 mg cefazolin, 80 mg amikacin, 50,000 U bacitracin and 500 ml 0.9% sterile saline (or a medically equivalent solution) immediately before implantation. Implant pockets were irrigated with the triple antibiotic solution before implantation or implant exchange.

Surgical Site Preparation

Animals were placed under general anesthesia. Anesthesia Procedure and positioned in dorsal recumbency (on back) on a surgery table. An ophthalmic lubricating ointment were administered to each eye. An orogastric “rumen” tube was inserted to prevent regurgitation aspiration and bloat during the anesthetized period. Animals were placed in dorsal recumbency on an operating gurney. The skin covering the mid-regions of both front limbs and the chest were close-clipped removing wool and hair by vacuum and scrubbed for aseptic surgery. Surgical scrubbing consisted of three (3), two-step cycles consisting of center-out scrubbing with a povidone iodine scrub solution and center-out iodine removal with 70% isopropyl alcohol. After a brief dry time, the scrubbed area was lightly sprayed with povidone iodine solution and allowed to dry. The animal was then be transferred to the surgical suite where a final scrub was performed and the surgical site was sterile draped for aseptic surgery.

Surgical Procedures

Test Articles Implantation

Each animal received a bilateral procedure and both sides were implanted with the same test article. A 10-20 cm incision through the skin and panniculus carnosus muscle was made along the ventral edge of one of the latissimus dorsi muscles; sequential or non-sequential bilateral procedures were performed. Soft connective tissue underneath the latissimus dorsi muscle was bluntly separated to create an implantation pocket of approximately 16-18 cm cranial-caudal by 12-15 cm dorsal-ventral to accommodate the tissue expander between the chest wall and latissimus dorsi muscle. The cranial edge of the implantation pocket was sutured to minimize void space and help prevent cranial implant migration. To cover the anterior gap created between the chest wall and latissimus dorsi muscle due to expander presence, test articles were trimmed to a size of approximately 5-7cm dorsal-ventral by 15-17cm cranial-caudal, with a flat edge for suturing to the latissimus dorsi muscle and a curved edge for suturing to the intercostal muscles to create an inframammary fold (IMF). The test article was first sutured to the ventral edge of the latissimus dorsi muscle, staggering the depth of suture bites into the muscle to reduce scaffold suture line pull out. Depth of the suture bite into the test article was maintained constant along the perimeter of the device in order to avoid unequal tensioning of the device within its structure. 2-0 absorbable suture (e.g. BIOSYN™) was used for the latissimus dorsi-scaffold suture line, in a simple continuous or continuous interlocking pattern. A 500-750 cc tissue expander, containing a portion of the total volume capacity of sterile saline solution, was inserted into the pocket, with the injection port positioned laterally and dorsally. The base of the tissue expander was positioned flat against the chest wall. The test article was then sutured to the lateral chest wall in a line approximating, but slightly posterior to, the anterior margin of the tissue expander.

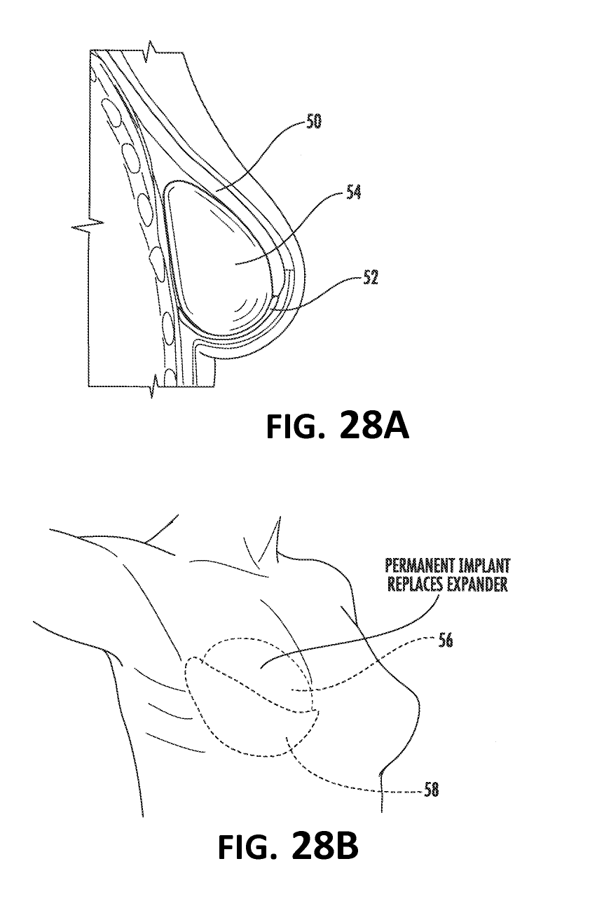

FIGS. 28A and 28B illustrate a human breast reconstruction. FIG. 28A illustrates a side view of a human breast with scaffold. In FIG. 28A, 32 is the pectoralis major muscle, 52 is the silk scaffold and 54 is a breast implant. FIG. 28B illustrates a permanent implant having replaced a tissue expander. In FIG. 28B, 58 is the silk scaffold (test article) and 56 is a breast implant.

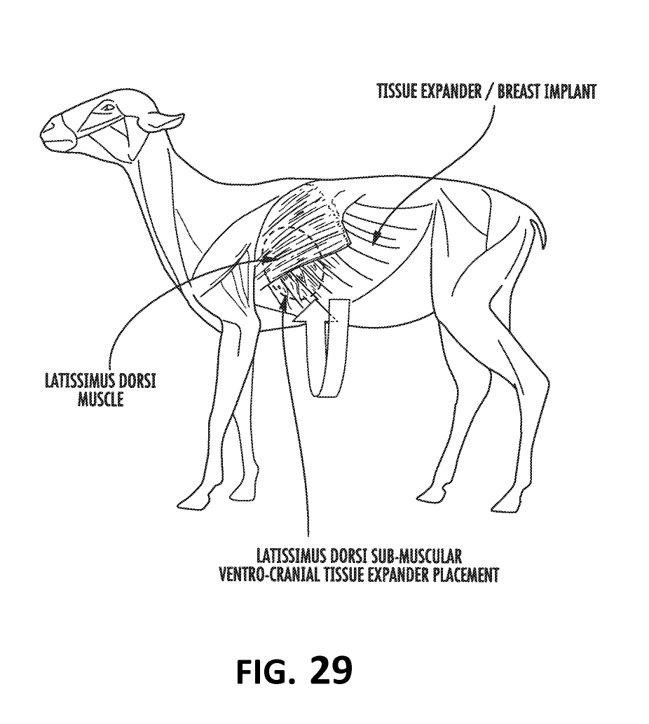

FIG. 29 illustrates a Latissimus dorsi sub-muscular tissue expander/breast implant location in sheep.

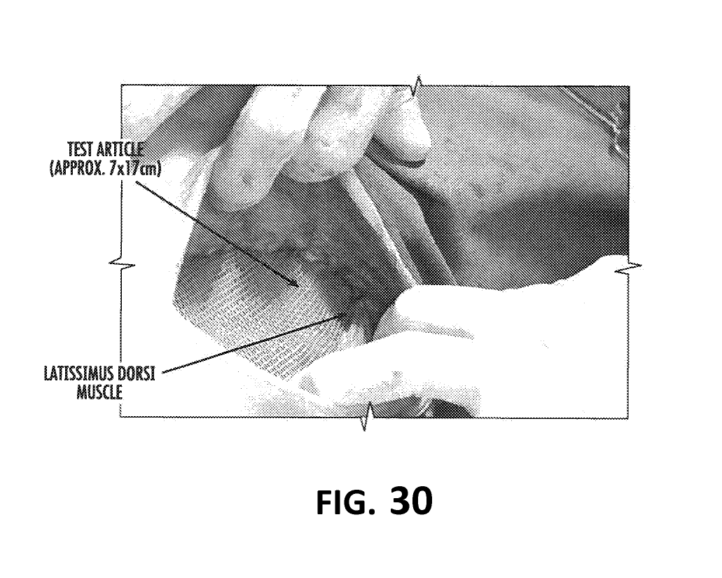

FIG. 30 is a photograph of test article placement upon completion of the surgical procedure prior to incision closing.

Interrupted “tackdown” suturing was performed at the cranial and caudal edges of the test article scaffold, and midway along the intended inframammary fold (IMF) line, to suspend the scaffold over the final hammock area. The IMF and corner tackdown stitches were of 2-0 absorbable suture (e.g. VICRYL®, BIOSYN™). Suturing was then performed along the intended IMF line in a simple or interlocking continuous pattern, using 2-0 absorbable suture (e.g. VICRYL®, BIOSYN™). Care was taken not to puncture the expander while suturing. The test article was optionally trimmed to the final size once suturing was complete and the tissue expander was optionally further filled to or beyond the target time 0 volume to reduce tissue expander folding. A temporary closed loop drain (BLAKE® Drain, Ethicon) was placed within the implant pocket (ventral to the IMF), and the tube portion of the drain system was tunneled subcutaneously from the pocket, dorsally for approximately 20 cm to exit the skin dorsal to the shoulder blade. The skin exit site for the drain tube was closed with 2-0 non-absorbable suture (e.g. PROLENE™), in purse string pattern, with French lace extension of suture strands up the exposed drain tube 2-4 cm to further secure the tube at the skin exit and minimize tube pistoning at the exit. The implantation surgical incision was closed in 2-3 layers as follows:

1. (Optional) Sub-pannicular soft connective tissue closure was optionally performed using 3-0 or 2-0 absorbable suture (e.g. VICRYL®, BIOSYN™) in a simple continuous pattern.

2. The panniculus muscle incision was closed in a simple continuous pattern with 2-0 absorbable suture (e.g. VICRYL®, BIOSYN™).

3. Skin margins were closed/apposed in a simple continuous subcuticular pattern with 2-0 or preferably with 3-0 absorbable suture (e.g. VICRYL®, BIOSYN™). All outer skin incision margins were sealed with liquid cyanoacrylate glue, and an antiseptic ointment or powder (e.g. nitrofurazone) was applied over the glued skin incision. Drain tubes were glued to the skin at the skin exit, and were optionally spot-glued along the tube path to connected suction bulbs that were secured to the skin/wool dorsal to the shoulder blades by suture and/or glue. The above procedure was performed for both sides of the animal either sequentially or simultaneously.

Sham controls

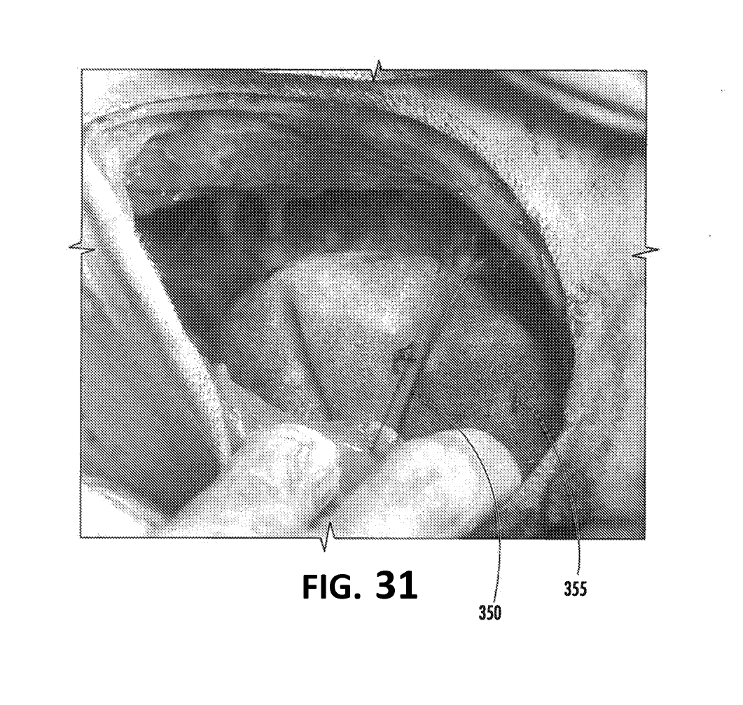

Sham control implantations followed the surgical procedure described above for ‘Test Article Implantations’, but rather than supporting the implanted tissue expander with a test article, the latissimus dorsi muscle was sutured to the chest wall (see FIG. 31) at three well separated locations over the tissue expander, spanning between the ventral free margin of the latissimus dorsi muscle and a chest wall arch consistent with IMF suture lines created in Test Article Implantations (Sham implants did not have IMF suture lines), using 2-0 absorbable suture (e.g. VICRYL®, BIOSYN™) in a mattress or cruciate pattern. In FIG. 31 item 350 shows the sutures (3 locations), and item 355 is the tissue expander.

Implant Exchange

Thirteen weeks (+2 weeks) following tissue expander implantation, expanders were surgically removed and replaced with the breast implant of corresponding size. This surgery was performed as follows, after previously described aseptic preparation of the surgery site and with the animal in sternal recumbency on the surgical table (bilateral simultaneous procedures were optionally performed): An 8-10 cm skin incision was made over the mid portion of the implanted expander, approximately in line with latissimus dorsi muscle fibers. The latissimus dorsi muscle was split in line with fibers by blunt and sharp dissection, taking care to not damage the tissue expander. The implanted tissue expander was atraumatically grasped and gently extracted from the pocket while stripping fibrous encapsulation away from the expander. The tissue expander was set aside in order to extract the following tissue expander samples for analysis on a scanning electron microscope (SEM) (samples did not need to be sterile):

1. 4×4 cm TE shell underneath the test article for SEM abrasion analysis; stored in 10% buffered formalin

2. 4×4 cm TE shell underneath the latisimus muscle for SEM abrasion analysis; stored in 10% buffered formalin

The pocket was inspected and irrigated with antibiotic irrigation solution, as previously described for Test Articles Implantation. A small biopsy specimen of the implanted test article (˜1 cm²) and the surrounding tissue was optionally excised from within the pocket. A breast implant of corresponding size to the final expander inflation volume was inserted into the vacant pocket, and the split latissimus dorsi edges was re-apposed in 2-3 layers as described previously for Test Articles Implantation incisional closure, with the following exception: the optional first/deepest layer was optionally used to oppose fibrous capsule incision margins—extracapsular—using 3-0 or 2-0 absorbable suture (e.g. VICRYL®, BIOSYN™) in a simple continuous pattern. Outer skin incision margins were sealed with liquid cyanoacrylate glue as previously described. A temporary drain was optionally placed in the surgical site. The above procedure was performed for both sides of the animal either sequentially or simultaneously.

Test article observations and measurements at the time of implant exchange procedures included:

Gross observations of pocket expansion (i.e., were there visible voids around the tissue expander/implant)

Position of the tissue expander/breast implant (e.g., any implant rotation, folding, etc.)

Visual assessment of ingrowth into the tissue expander at time of exchange

Visual assessment of interior surface of device (e.g., device visible, ingrowth removed from TE pores visible, etc.) at time of exchange

Volume and type estimate of fluid within pocket at time of exchange

Post operative observations following the implant exchange surgeries mimicked those performed post-implantation in terms of frequency and data collected.

Tissue Expander Fillings

The tissue expander was filled to a percentage of its total volume at the time of implantation. The remaining volume was divided into multiple clinically appropriate fills. During the saline injections, a degree of blanching was optionally observed by the research facility veterinarian. If blanching occurs, the research facility veterinarian optionally reduced the injection volume accordingly, the deviation was documented on the recording forms. Animals were sedated for tissue expander filling. Study-specific animal observations were documented.

Postoperative Assessment

Tissue Expander and Breast Implant Positioning

The following tissue expander positioning measurements were taken at the time of the implantation surgery, at each postoperative tissue expander filling, and at monthly increments from the time of implantation. Additionally, these measurements were taken directly before the exchange of the tissue expander for the breast implant and one week following the exchange. The results were recorded.

Tissue Expander Vertical Positioning

Girth about Thoracic Region Cutting Axially Through Implant (Referred to as “Girth”)

Distance from Spine to Ventral Margin of Implant (Referred to as “Spine to implant”)

Tissue Expander Horizontal Positioning

Distance from Base of Tail to Shoulder (Referred to as “Tail to Shoulder”)

Distance from Base of Tail to Caudal Margin of Implant (Referred to as “Tail to implant”)

In addition to these measurements, photographs were taken to indicate the location of the tissue expander fill port at implantation, each tissue expander filling and/or at monthly intervals until the breast implant exchange procedure was completed.

Palpability

Device palpability through the skin was assessed at time of surgery, tissue expander fillings and at one month intervals post-operatively. At necropsy, palpability was assessed with the skin and then without the skin and over the panniculus muscle. Palpability was assessed by pressing firmly on the lower pole of the breast and observations recorded. Palpability was scored as follows: 0 meant device could not be felt; 1 meant device suture lines could be felt, but individual features (pores, etc.) could not be discerned; 2 meant device features could be felt (e.g., pores, wrinkles, etc.) but were not visible; 3 meant device features were visible through muscle and easily discerned.

Diagnostic Imaging

Ultrasonographic imaging was performed for animals in the 12 month group, but also was optionally performed for 18 and 24 month groups. Images were taken at the following time points: 1) directly prior to the first tissue expander filling 2) at the time of the last tissue expander filling, 3) 3 months post-operative, 4) 6 months post-operative and 5) 12 months post-operative. Additional ultrasonographic imaging was optionally performed on an as needed basis for animals experiencing adverse events.

Computed tomography (CT) scanning and magnetic resonance imaging (MRI) was performed for animals in the 12 month group, but also was optionally performed for 18 and 24 month groups. Images were taken at 6 and 12 months post-operative. Additional CT and/or MRI data was optionally requested. The imaging output was captured and saved.

Necropsy

Following euthanasia and gross examination of the external animal body, the surgical site was prepared for asepsis in the same manner as it was prepared for the device implantation, except that the animal was placed in a ventro-lateral recumbency on the surgical table. A single surgical site at a time was examined (one side of one animal preceding the other). A broad section of the skin (approximately 20×20 cm) covering the implantation site and the immediate surrounding area was extracted to expose the panniculus muscle. The health of the panniculus muscle was evaluated (inspecting for hematoma, blanching, etc.) and the palpability of the scaffold through the panniculus was assessed. Culture swabs were optionally taken in areas of interest if infection was suspected. The entire 20×20 cm complex from the rib cage and surrounding tissue was released with minimal handling and laid on the sterile back table so that the medial or deep side of the TE was facing up (panniculus muscle was against the table). If infection was suspected, the test article was optionally approached dorsally to excise a sample approximately 0.5×0.5 cm through the capsule and lat muscle for infection analysis. In the same manner, two additional infection samples were cut out on the cranial and caudal sides of the test article. Once all infection analysis samples were taken the tissue expander was deflated and the examination table was no longer considered a sterile field. Only after the examination table was no longer considered sterile preparation for necropsy begin on the next scheduled site. After all saline was been drained from the tissue expander the dorsal hemisphere of the tissue expander was bisected to separate the upper anterior tissue expander hemisphere from the posterior hemisphere, leaving the ventral hemisphere of the tissue expander intact and in contact with the implanted device. The upper half of the posterior tissue expander hemisphere was excised and a 4×4 cm sample of the attached capsule was removed for burst testing and a 2×3 cm sample of the shell and adhered capsule was cut for histology. The remaining burst and histology samples that contain the tissue expander shell, capsule, and implanted scaffold was dissected using a scalpel to collect the samples. A 4×4 cm sample of the capsule adhered to the tissue expander underneath the latissimus dorsi muscle was excised and the 4×4 cm shell removed from the back of the scaffold burst sample was retained and stored for SEM analysis.

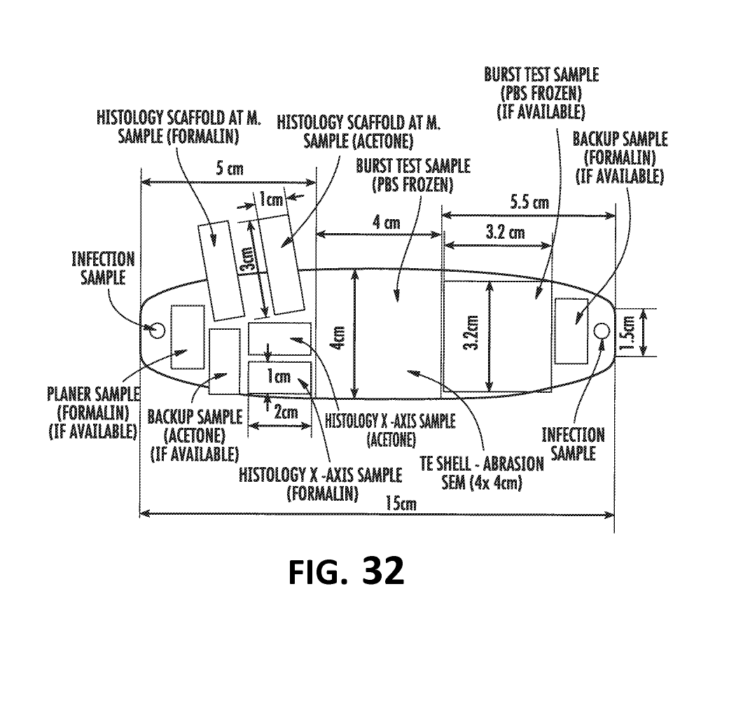

FIG. 32 illustrates a layout of biomechanical and histological samples extraction from each test article. Additionally, necropsy observations and measurements at 1, 3, 6, and 12 months included: (1) appearance, integration, and size of the tissue encapsulating the test article; (2) adhesion of the tissue into the structure of the test article; (3) visual inspection of tissue expander/breast implant. Additional samples were collected if determined to be of interest.

Bioresorption—Morphology/Morphometric Analysis

Samples of the tissue expander and breast implant were excised from the regions where implants were in contact with both the test article and muscle for comparison. Layout of biomechanical and histological samples extraction from each test article was shown in FIG. 32

This study sets forth a method for determining suitability of an implantable silk scaffold for use in human soft tissue repair or support, the method comprising the step of implanting a silk scaffold in a quadruped. The quadruped can be a sheep or a pig. We determined that the silk scaffold can maintain at least 90% of its time zero strength at one month, three months and at six months in vivo after implantation. And we determined that the silk scaffold can substantially maintain its time zero (i.e. at time of implantation) strength throughout its duration in vivo. Additionally, the thickness of the scaffold can increase with time in vivo due to tissue ingrowth. The scaffold was implanted to simulate implantation in a human breast reconstruction or augmentation procedure. The scaffold can be implanted without regard to side orientation of the scaffold.

To summarize, in this Example various embodiments of SeriScaffold™ a unique silk-derived, long-term bioresorbable scaffold medical device (the “device”) were studied. SeriScaffold™ can be used for example to provide soft tissue support in various surgical procedures, such as breast reconstruction. In this Example a 2-stage implant-based breast reconstruction model was developed in sheep to characterize over a twelve month period biomechanical and clinical properties of the device. Thus, in a pectoralis muscle elevation procedure (as often used in human breast implant breast reconstruction procedures) tissue expanders (TE) were implanted bilaterally under the elevated latissimus dorsi (LD) muscle of Rambouillet cross or Suffolk-Hampshire cross sheep. The device provided soft tissue support resulting in an inframammary fold (IMF) in the “lower pole” between the LD and the chest wall. Three animals each were euthanized at 1, 3, 6, and 12 months. The animals slated for 6- and 12-month analyses underwent a second surgery at 13+2 weeks post-op to exchange the TE for a breast implant (BI). At necropsy, periprosthetic tissue samples containing the device were collected and biomechanical strength was assessed using a standard ball-burst test and drapability of the samples was rated minimal, moderate, or significant. In-life clinical characterization included assessments of fluid collection, capsular contracture, and device palpability. At each time point, at least six samples were obtained for biomechanical characterization. At all time points, the device pore areas were fully ingrown with tissue that had infiltrated into all device surfaces (see FIGS. 35 and 36). The thickness of the samples increased from 0.9±0 mm at time=0 to 1.9±1.3 mm at 1 month and 2.2±1.0 mm at 12 months. As initial scaffold (device) strength decreased due to bioresorption, tissue ingrowth contributed to device strength with an ultimate burst load of 153±69 N at 1 month and 243±83 N at 12 months. Clinically, no evidence of capsular contracture >Baker grade 2 was observed and all explanted samples were rated as significantly drapable at all time points. Drain output yielded an average of 48±10 mL/24 hrs per implant site, with a maximum yield of 132 mL. Drains were in place for 3 days in 8 animals, and 4 and 5 days in 2 animals each. The device was not palpable through the skin at any time point. This Example sets forth the first successful use of a sheep model for simulation of full-scale human implant-based breast reconstruction with satisfactory soft tissue support resulting in an IMF using the device, a unique new silk-derived surgical scaffold. The biomechanical strength profile of the device over 12 months indicated consistent soft tissue support. Clinically, the tissue around the TE/BT was soft, supple, and drapable with no evidence of capsular contracture.

Thus this study sets forth a method for evaluating in vivo a medical device in a quadruped animal model, the method comprising the step of implanting a quadruped with a tissue expander and a silk scaffold to support soft tissue. This method can comprise suturing the silk scaffold to a sub-latissimus dorsi muscle and a chest wall of the quadruped.

Additionally, this study set forth an animal model system for determining suitability of an implantable silk scaffold for use in human soft tissue repair, the animal model system comprising a silk scaffold, and a quadruped having a muscle for providing internal support for the silk scaffold. The quadruped can be is a sheep or a pig and the muscle can be a sub-latissimus dorsi muscle.

This sheep study (Example 1) determined that the silk scaffold (SeriScaffold™) is well suited for use in human breast reconstruction surgery and procedures and the results from this sheep study showed that the silk-based devices or scaffolds of the present invention are highly desirable materials to use in breast reconstruction and breast augmentation procedures in humans, as well as for other human organ and implanted medical device support purposes.

The sheep model was used to determine suitability of SeriScaffold™ for use in human breast implantation. SeriScaffold™ is a bioresorbable silk-derived surgical scaffold, for the provision of soft tissue support. The sheep model emulates the mechanical and biological environment of a two stage breast reconstruction using SeriScaffold®, a unique silk-derived bioresorbable scaffold (SBS) developed to provide soft tissue support. The sheep model also evaluated the clinical, mechanical and biological performance of the SBS over 12 months in vivo. SBS is bioresorbed in-vivo over a 12 month period after implantation during which native neovascularized tissue develops in its place. This sheep model was developed to characterize the ability of SBS to provide soft tissue support in breast reconstruction. In this study twelve sheep underwent bilateral implantation of tissue expanders under the latissimus dorsi muscles with SBS sutured between the latissimus dorsi and the chest wall. The SBS was sutured between the latissimus dorsi and the chest wall creating an infra-mammary fold (IMF) and defining a soft tissue lower pole. Animals were evaluated 1, 3, 6 and 12 months post-surgery. Animals designated for the 6 and 12 month endpoints experienced a second surgery after 3 months to exchange the tissue expanders for permanent breast implants. Implant sites of each animal were imaged throughout the study using CT and MRI. At necropsy, SBS (with or upon removal of ingrown or newly regenerated tissue) thickness and drapability were recorded. Biomechanical strength of tissue samples (with at the time of observation any remaining non-bioresorbed SBS) was assessed using a standard ball-burst test. The results of this study were as follows: SBS was not palpable at any time point. There were no cases of implant migration. The position of SBS was visualized by MRI out to 6 months. SBS pore areas were fully ingrown with new, native tissue from +1 month after implantation (initial surgery) and thereafter. The SBS devices were drapable (see FIGS. 35 and 36). The thickness of samples of SBS (with ingrown or newly regenerated tissue) increased from 0.9±0 mm at time=0 to 1.9±1.3 mm at +1 month and to 2.2±1.0 mm at +12 months. The burst load of samples of implanted SBS (with ingrown or newly regenerated tissue) increased from 153±69 N at +1 month to 246±83 N at +12 months. Burst testing (material strength) performed on SBS samples with the ingrown or newly regenerated tissue removed resulted in burst loads of 98±35 N (at +1 month), 30±11 N (at +3 months), and 7±2 N ( at +6 months), showing progressive resorption of the SBS; no load was calculated for +12 month SBS samples, because the SBS had by then bioresorbed. This study showed that the sheep model of implant-based breast reconstruction was a satisfactory means of evaluating SBS for use in human breast reconstruction. Furthermore, this study showed that the strength of regenerated tissue was not only maintained, but increased over time. As the strength of the implanted SBS decreased, the strength of the SBS samples with ingrown or newly regenerated tissue increased, showing therefore a progressive transfer of load-bearing responsibility to the newly regenerated tissue. Thus, the SBS is a bioresorbable device with the ability to provide soft tissue support in breast reconstruction.

### Example 2

### Study of Tissue Expander with Silk Scaffold in Pig

An experiment was carried out using a mini pig cadaver lab. The pig was a Yukatan Mini Pig about 18 months old and weighing about 91 kg. The scaffold used in this experiment was the Silk-Based Device No. 1, 10×25 cm, a device within the scope of the present invention (SeriScaffold™). The tissue expander used in this pig study was the NATRELLE Style 133MV 500cc, Model No. 133MV-14

The pig was euthanized for animal model and surgical procedure development. A breast reconstruction procedure was simulated and performed using a sub-latissimus dorsi tissue expander implantation. An incision was made through the skin and the adipose tissue approximately 2-3 cm ventral from the latissimus dorsi muscle. The latissimus dorsi muscle was separated and elevated from the underlying serratus ventralis and the tissue expander was inserted in the sub-muscular pocket formation. The surgical scaffold was sutured to the ventral edge of the latissimus dorsi muscle and to the chest wall to support the tissue expander in a sub-muscular position. The tissue expander was then filled to its maximum capacity in several stages during which the resulting tension on the scaffold, sutures, and surrounding tissue was observed. In addition, pectoralis muscles was an alternative sub implantation site.

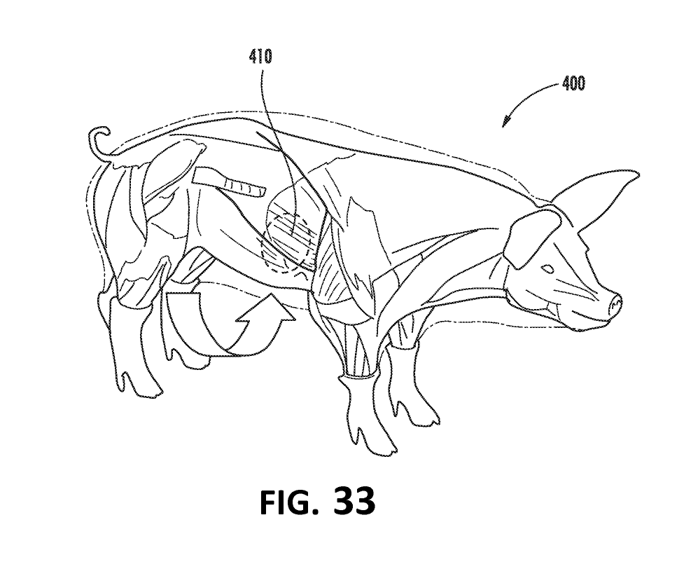

The latissimus dorsi muscle was easily identified and elevated. The thickness of the muscle was found to be adequate for suturing the scaffold to its ventral edge. The size of the sub-muscular pocket was sufficient for placement of a 500 cc tissue expander. The skin incision placement was optimized for access to the ventral edge of the latissimus dorsi muscle. After the tissue expander was filled to its full capacity, scaffold, sutures, and the surrounding tissue supported the imposed tension. An excessive layer of subcutaneous adipose tissue (approx. 2″) was observed. FIG. 33 illustrates a Latissimus Dorsi sub-muscular ventro-cranial tissue expander placement in a pig for modeling in a human. In FIG. 33 item 400 is the pig and item 410 is the pig's latissimus dorsi muscle.

### Example 3

### Use of the Silk Scaffold in Human Breast Reconstruction and/or Augmentation

A tissue expander can be placed adjacent to the pectoralis major muscle of a female human patient and positioned under the muscle. A test device, SMB Nos, 1, 2, 3, 4, 5, or 6, surgical scaffold (SeriScaffold™), can be sutured to the pectoralis muscle and chest wall to support the soft tissue covering the tissue expander. The tissue expander and muscle can be supported by placing sutures between the muscle and the chest wall. The procedure can be performed unilaterally or bilaterally on the right and/or left side of each female patient. The tissue expander can be filled with saline to capacity over time and a stage TI surgical procedure subsequently performed. A Stage II surgery can consist of removal of the tissue expander and placement of a breast implant. The silk scaffold can also be used is breast augmentation surgeries and procedures (where a tissue expander is typically not used) by suturing the silk scaffold in a position so that it will support the lower pole or end of a breast implant to thereby prevent undue movement of the breast implant post implantation and to support tissue ingrowth as the silk scaffold bioresorbs.

In another aspect of the invention, a silk-derived, bioresorbable scaffold device for soft tissue support, evaluated in a sheep model simulating human breast reconstruction, is provided.

The device is useful as a transitory scaffold for soft tissue support, for example, where weakness or voids exist that require the addition of material to obtain the desired surgical and aesthetic outcome.

Concerns within the aesthetic surgery field that are addressed by the present device include, but are not limited to: seroma, infection and high rates of explant, palpability/scar encapsulation. The present devices are directed at reducing and/or eliminating at least some of these concerns.

The present device is useful for at least the following applications: face lift, eyelid lift, gingival grafting, neck lift, breast augmentation and reconstruction, breast revision augmentation, mastoplexy, correction of genetic disorders of the breast, hernia repair including inguinal hernia, abdominal repair, including TRAM flap, abdominoplasty for MWL, ventral hernia, hernia prophylaxis.

The device comprises a silk-derived bioresorbable scaffold (SBS). It may be a knitted, cuttable, long-term bioresorbable scaffold as described elsewhere herein.

As shown in FIG. 37, a full-scale animal model, as described elsewhere herein, developed for testing the presently described devices to simulate two stage breast reconstruction, are provided. The animal model has demonstrated the effectiveness of the present devices for soft tissue support.

As shown in FIG. 38, neovascularization and ingrowth was observed intraoperatively at 3 months tissue expander, breast implant exchange. The device showed with continuous support out to 12 months.

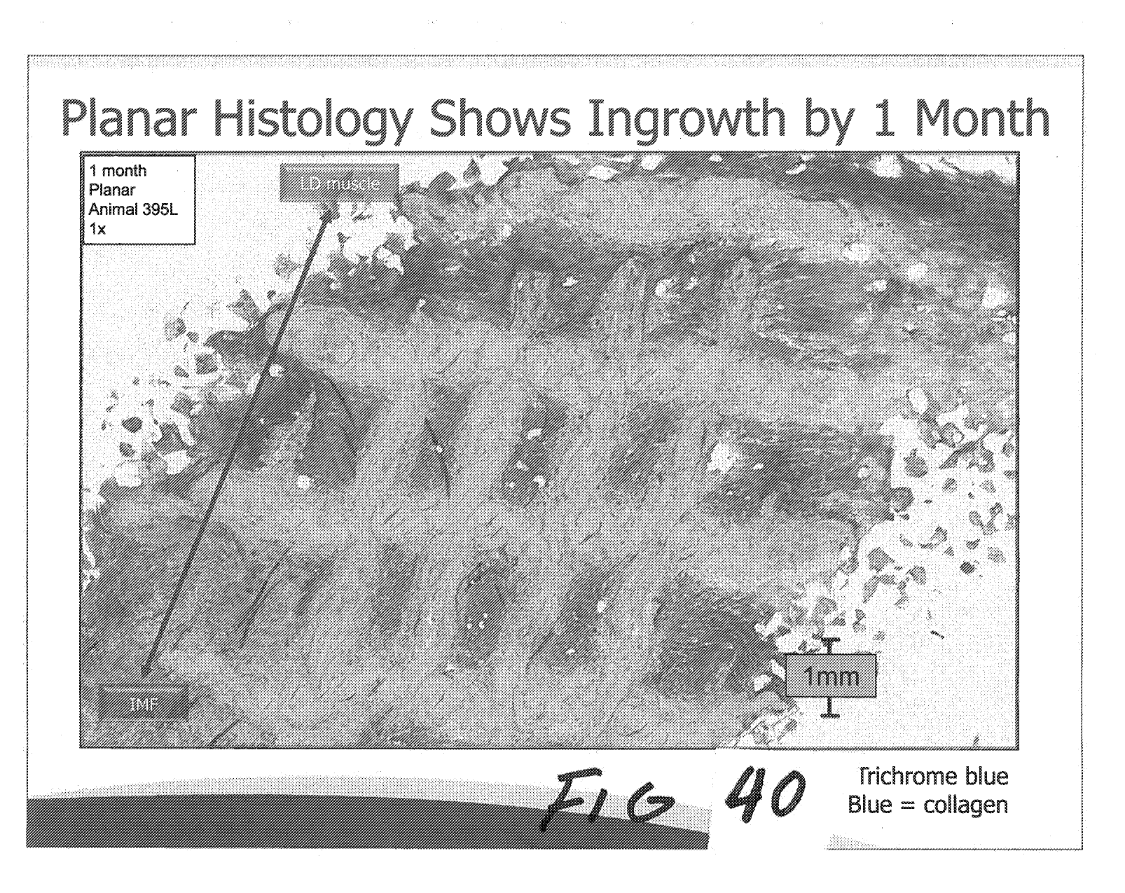

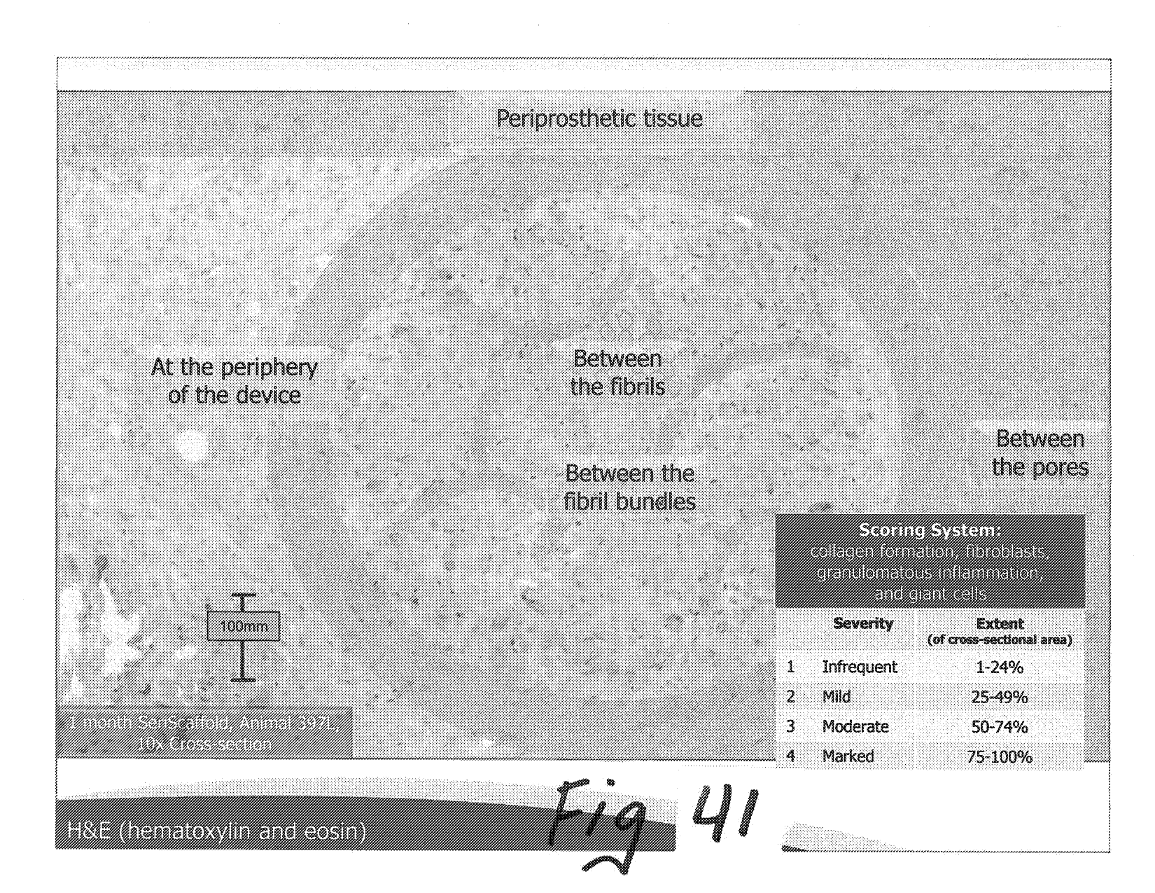

As shown in FIG. 39, native tissue generation was facilitated by the device. Planar histology shows tissue ingrowth by one month from implant, as shown in FIG. 40.

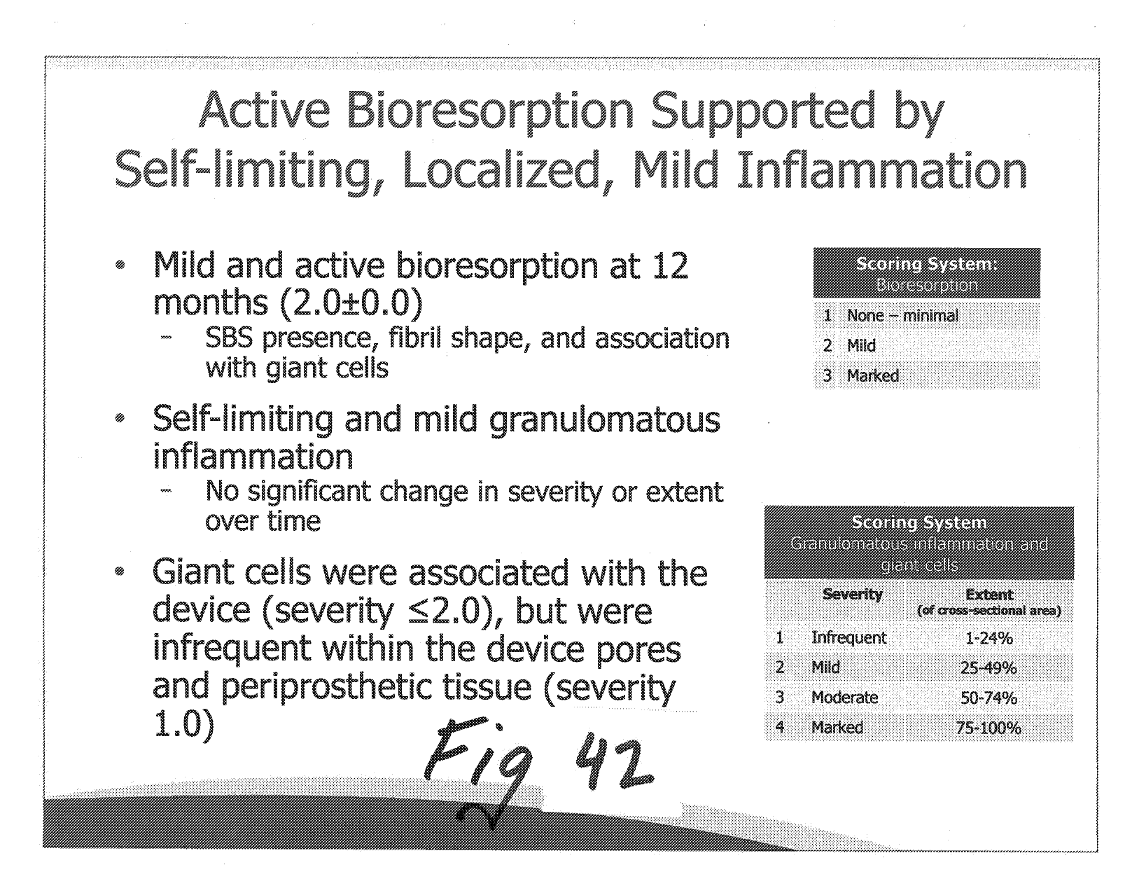

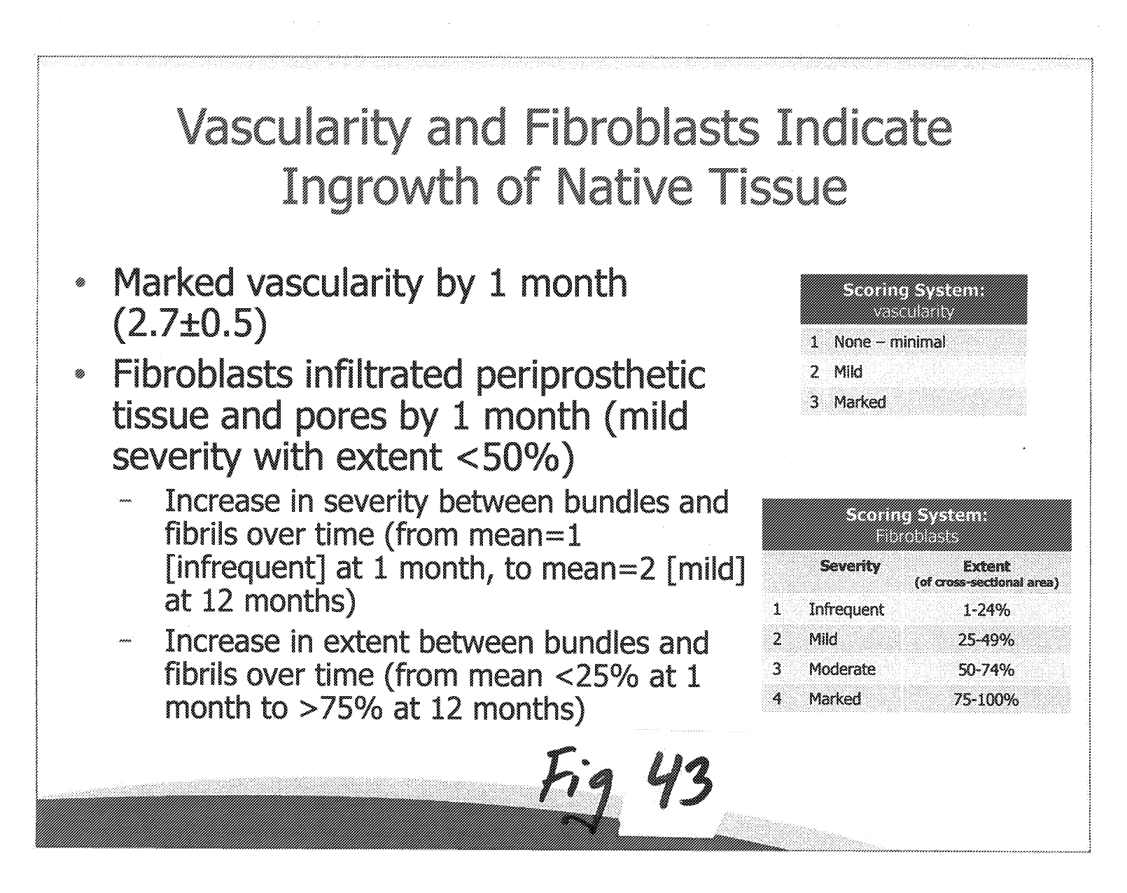

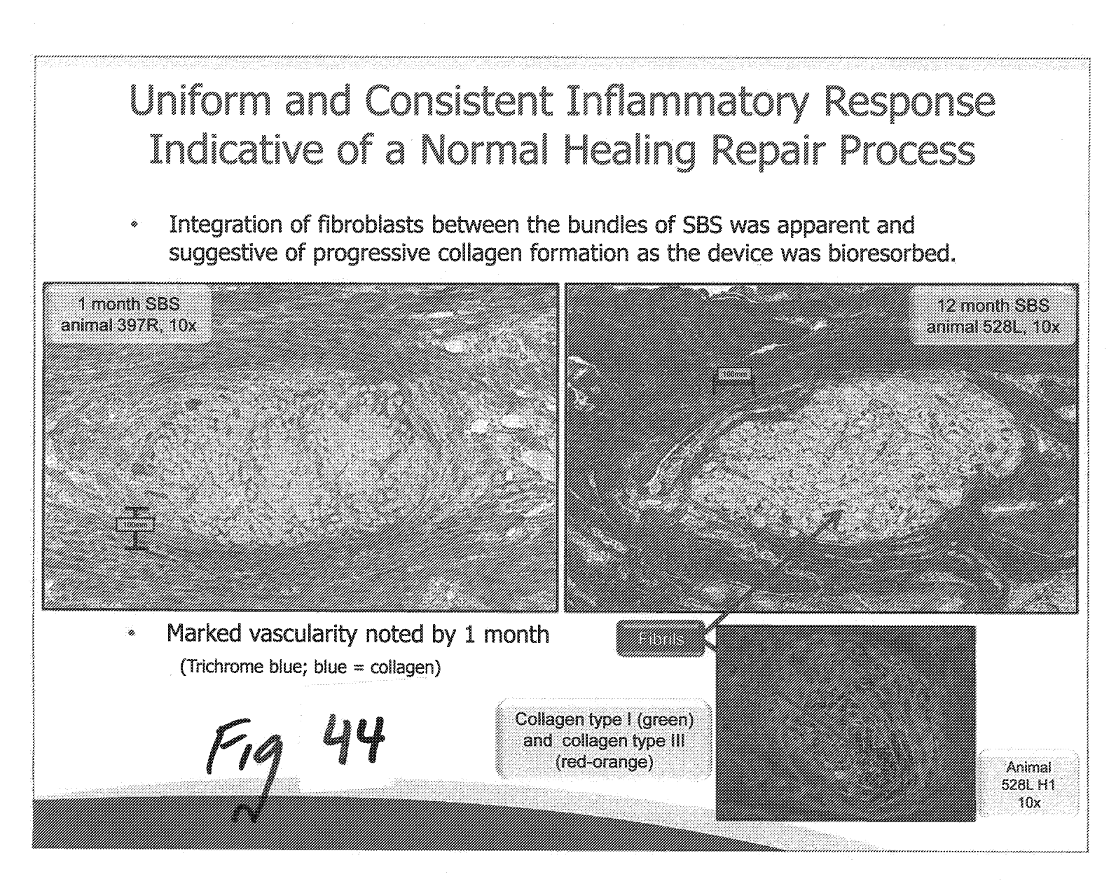

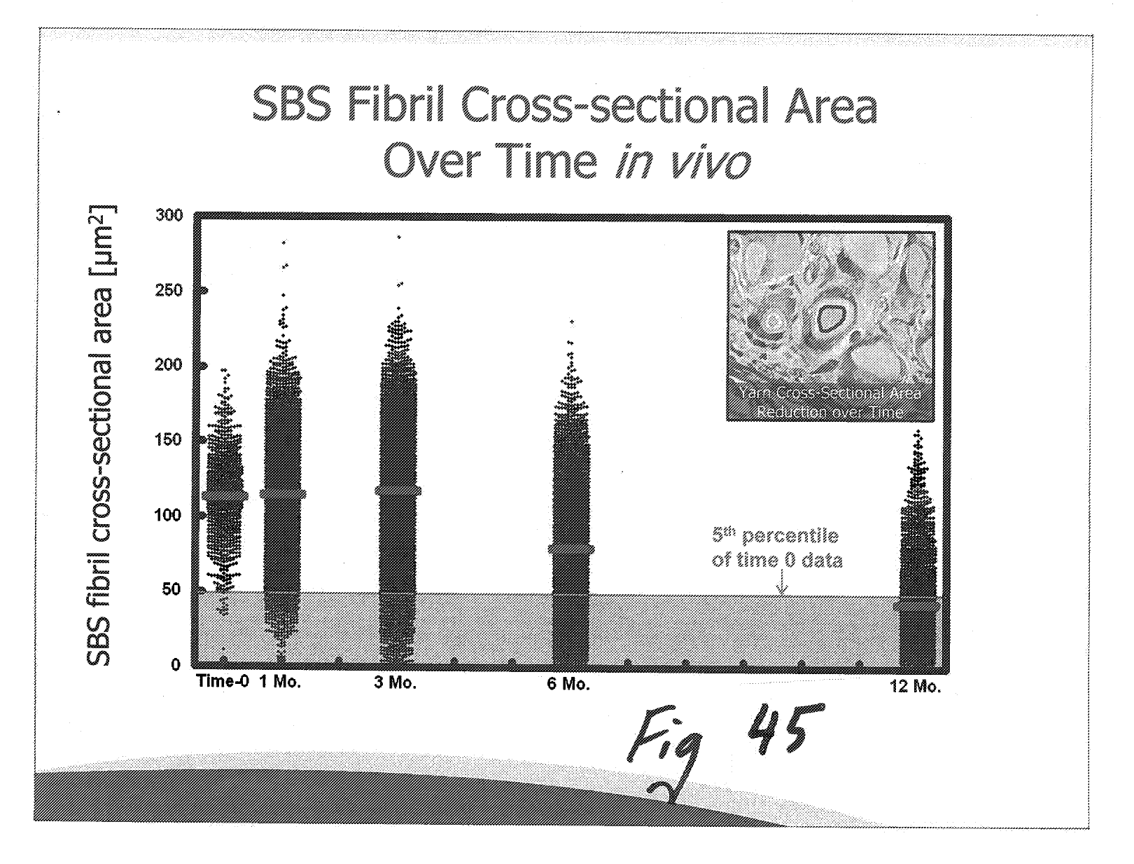

Other observations and scoring with respect to tissue response to the implanted devices are shown in FIGS. 41-45.

Many of the present devices and methods provide tissue enhanced tissue support, complete integration with tissue generation and vascularity, a normal healing response (comparable to the use of conventional sutures for example), collagen formation that is primarily type I collagen at 12 months from implant, improved tissue adherence, and a predictable bioresorption.

Many embodiments and adaptations of the present invention other than those herein described, as well as many variations, modifications and equivalent arrangements, will be apparent from or reasonably suggested by the present invention and the foregoing description thereof, without departing from the substance or scope of the present invention. While the present invention has been described herein in detail in relation to its preferred embodiment, it was to be understood that this disclosure was only illustrative and exemplary of the present invention and was made merely for purposes of providing a full and enabling disclosure of the invention. The foregoing disclosure was not intended or to be construed to limit the present invention or otherwise to exclude any such other embodiments, adaptations, variations, modifications and equivalent arrangements.

What is claimed is:

1. A method for supporting tissue in breast augmentation or breast reconstruction surgery, the method comprising the steps of:
implanting, in an inframammary fold of a patient, a silk-derived bioresorbable scaffold device, the device providing sustained tissue support for at least 12 months from the date of implant.
